(19) United States
(12) Patent Application Publication
(10) Pub. No.: US 2004/0177388 A1
(43) Pub. Date: Sep. 9, 2004

(54) Methods and compositions for the identification and treatment of neurodegenerative disorders

(75) Inventors: Juan Botas, Houston, TX (US); Huda Zoghbi, Houston, TX (US); Pedro Fernandez-Funez, Houston, TX (US)

Correspondence Address: BOWDITCH & DEWEY, LLP, 161 WORCESTER ROAD, P.O. BOX 9320, FRAMINGHAM, MA, 01701-9320, US

(73) Assignee: Baylor College of Medicine

(21) Appl. No.: 10/291,871

(22) Filed: Nov. 8, 2002

Related U.S. Application Data
(63) Continuation of application No. 10/017,761, filed on Oct. 29, 2001, now abandoned.
(60) Provisional application No. 60/244,101, filed on Oct. 27, 2000.

(51) Int. Cl. A01K 67/033

(52) U.S. Cl. 800/8

(57) ABSTRACT

The present invention relates to Drosophila models of the neurodegenerative disorder spinocerebellar ataxia 1 (SCA-1). In particular, the invention relates to transgenic Drosophila which express normal human ataxin-1 or mutant human ataxin-1 with expanded polyglutamine repeats for SCA-1 therapeutics. The invention further relates to the diagnosis of predispositions to developing SCA-1. The invention further relates to methods of using the transgenic Drosophila to screen for therapeutics of SCA-1 and other neurodegenerative disorders. The invention further relates to the identification of modifier genes of the SCA-1 phenotypes produced by overexpression of ataxin-1, for therapeutic and diagnostic uses and for screening for therapeutics of SCA-1 and other neurodegenerative disorders. The invention further relates to the diagnosis of a predisposition to SCA-1 comprising detecting the overexpression of normal ataxin-1.

This application claims priority to U.S. Provisional Application No. 60/244,101, filed Oct. 27, 2000, which is incorporated by reference herein in its entirety.

This invention was made with government support under Research Grant 5 R01 GM55681 from the National Institutes of Health. The government has certain rights in the invention.

### 1. FIELD OF THE INVENTION

The present invention relates to Drosophila models of neurodegenerative disorders, more particularly polyglutamine-induced neurodegenerative disorders, and more particularly to spinocerebellar ataxia 1 (SCA-1). In particular, the invention relates to transgenic Drosophila which misexpress normal human ataxin-1 or a mutant human ataxin-1 with expanded polyglutamine repeats. The invention further relates to methods of using the transgenic Drosophila to screen for therapeutics of neurodegenerative disorders, and in particular therapeutics of polyglutamine-induced neurodegenerative disorders, including but not limited to SCA-1. The invention further relates to the identification of modifier genes of ataxin-1 misexpression, for therapeutic and diagnostic uses and for screening for therapeutics of neurodegenerative disorders. The invention further relates to the diagnosis of a predisposition to SCA-1 comprising detecting the overexpression of normal ataxin-1.

### 2. BACKGROUND OF THE INVENTION

Neuropsychiatric and neurodegenerative disorders are beginning to be understood at the molecular level. Neurodegenerative disorders are typically characterized by a number of neuropathological abnormalities, such as neuritic plaques, neurofibrillary tangles (NFTs), Lewy bodies, and nuclear inclusions. Strikingly similar pathologies commonly associated with the neurodegenerative disorders can be arrived at by a large number of different genetic mechanisms. For example, a pathogenic mutation in the prion gene results in both tangle and Lewy body pathologies of prion disease (Feany and Kickson, 1995, Am. J. Pathol. 146: 1388). Mutations in tau protein lead to dementia in frontotemporal dementia (Hutton et al., 1998, Nature 393: 702) in addition to neurofibrillary tangles; mutations in synuclein lead to the presence of Lewy bodies and Parkinson's disease (Polymeropoulos et al., 1997, Science 276: 2045).

Many of the pathologies associated with neurodegenerative disorders are caused by gain of function mechanisms in which the relevant protein is altered, becomes toxic to the cell, and aggregates (Kaytor & Warren, 1999, J. Biol. Chem. 274:37507-10). Among these so-called “proteinopathies” are Alzheimer's disease, Parkinson's disease, prion disorders, and the polyglutamine diseases.

Alzheimer's Disease is a neurodegenerative disorder of the elderly that results in dementia and, ultimately, death. The physical alterations in the brains of diseased individuals are both intracellular, manifested as neurofibrillary tangles consisting of 10 nm paired helical filaments (PHFs); and extracellular, manifested as amyloid plaques surrounding nerve terminals. Other physical changes may include microvascular amyloidosis and dystrophic cortical neurites (for a review on the pathological hallmarks of AD, see Sobow, 1996, Folia Neuropathol. 34:55-62). The components of the two main types of lesions are known. Neurofibrillary tangles consist of the intermediate filament protein Tau. In healthy neuronal tissue, Tau is an unphosphorylated protein but is found to be phosphorylated in PHFs. Arnyloid plaques, also called senile plaques, consist of amyloid-β-peptide (Aβ), a product of the cleavage of an amyloid precursor protein (APP). In normal individuals, most of Aβ is in a 40-amino acid form; in addition, there exist minor amounts of Aβ that is 42 amino acids in length. In individuals with Alzheimer's Disease, the 42 amino acid form of Aβ predominates. The extent to which neurofibrillary tangles and amyloid plaques are present in the brain corresponds to the degree of senility caused by Alzheimer's Disease. Thus, a common theme in Alzheimer's Disease pathology is the production of aberrant structures that are not normally found in healthy brains. This observation is supported by the finding that PHFs are associated with ubiquitin, which would normally lead to the degradation of the associated molecule but in Alzheimer's Disease does not do so.

Polyglutamine repeat diseases, also known as triplet repeat diseases, are caused by expansion of unstable CAG repeats coding for glutamine within the respective proteins. The proteins implicated in these diseases—spinocerebellar ataxias (SCA) 1, 2, 6 and 7, Machado-Joseph disease (MJD, also known as SCA3), Huntington Disease (HD), spinobulbar muscular atrophy (SBMA), and dentatorubropallidolusyan atrophy (DRPLA)—are not known to be related to each other except that they all contain polyglutamine repeats. In addition they typically cause degeneration only in a specific subset of neurons (Zoghbi & Orr, 2000, Annu. Rev. Neurosci. 23: 217-247).

The development of mouse model systems for SCA-1, SCA-3, Huntington, and DRPLA has contributed enormously to our understanding of the polyglutamine diseases. They confirmed that pathogenesis follows expression of the expanded transgenes, and is not a consequence of lack of function of the normal proteins (Burright et al., 1995, Cell 82:937-48; Ikeda et al., 1996, Nat Genet 13:196-202; Mangiarini et al., 1996, Cell 87:493-506; Matilla et al., 1998, J Neurosci 18:5508-16; Lin et al., 1999, Neuron 24:499-502; Schilling et al., 1999, Neuron 24:275-86). Furthermore, polyglutamine proteins appear to exert their toxic effects in the nucleus; nuclear import correlates with pathogenesis even when the normal protein is cytoplasmic, as in the case of ataxin-3. This idea was tested in transgenic mice producing an expanded ataxin-1 protein that carries a mutation in the nuclear localization signal (Kiement et al., 1998, Cell 95:41-53). The protein was prevented from entering the nucleus and therefore could not initiate SCA-1 pathogenesis. Experiments adding a nuclear export signal to an N-terminal fragment of huntingtin in a cellular model proved the importance of nuclear localization for huntingtin toxicity (Saudou et al., 1998, Cell 95:55-66).

Mouse studies have yielded insight into another common feature of polyglutamine diseases, the formation of nuclear inclusions (NI)—aggregates of the expanded protein, or, in some cases, cleavage products containing the expanded polyglutamine tract. It was first believed that the NI were responsible for SCA-1, but a SCA-1 mouse model having an expanded ataxin-1 with a deletion in its self-association domain developed SCA-1 pathology without forming NI (Kiement et al., 1998, Cell 95:41-53). This observation suggested that NI formation is not required for the initiation of disease, and similar conclusions were made from a cell culture assay for huntingtin (Saudou et al., 1998, Cell 95:55-66). Besides accumulating mutant protein, the NI also accumulate molecular chaperones, ubiquitin and the proteasome (reviewed in Kaytor and Warren, 1999, J Biol Chem 274:37507-10). This discovery suggested that the NI might not be pathogenic at all, but rather reflect the cell's effort to free itself from the altered proteins. To test the possibility that the breaking down of the proteolytic pathway contributes to the disease, SCA-1 mice were crossed with mice lacking a ubiquitin-protein ligase. The offspring had fewer NI, but greatly accelerated pathology (Cummings et al., 1999, Neuron 24:879-92). These findings, together with the observation that NI can also occur in neurons that do not degenerate, suggest that the NI may actually benefit the cell by sequestering the toxic protein.

Notwithstanding the success of mouse models, Drosophila has several advantages that make it an appropriate model for neurodegenerative disorders caused by gain of function mechanisms. The GAL4/UAS system (Brand and Perrimon, 1993, Development 118:401-415) allows control of transgene expression. Transgenic lines that are silent (i.e., do not express the toxic transgene on their own) can be generated, and then crossed to a variety of GAL4 driver lines that direct expression to different cell types or even to specific neurons. Furthermore, altering the culture temperature modulates the amount of expression for a given transgenic line. Most of the genetic pathways involved in normal development and disease conditions are conserved between Drosophila and mammals. Mechanisms of neuronal degeneration in Drosophila, therefore, will likely prove relevant to neurodegeneration in humans. In support of this idea, Drosophila models using polyglutamine or truncated polypeptides of the MJD and Huntington proteins (Wamck et al., 1998, Cell 93:939-49; Jackson et al., 1998, Neuron 21:633-42; Kazemi-Esfajani and Benzer, 2000, Science 287:1837-40; Marsh et aL, 2000, Hum Md Genet 9:13-25) show the characteristic progressive neural degeneration preceded by NI formation (Wamck et al., 1998, Cell 93:939-49; Jackson et al., 1998, Neuron 21:633-42). The value of the Drosophila system was underscored by demonstrating that overproduction of the Hsp70 and Hsp40 molecular chaperones suppressed polyglutamine-induced neurotoxicity (Warrick et al., 1999, Nat Genet 23:425-8; Kazemi-Esfarjani and Benzer, 2000, Science 287:1837-40). This observation followed previous experiments in tissue culture showing that overexpression of chaperones reduced aggregation, but it was not known whether this had any effect on pathogenesis (Cummings et aL, 1998, Nat Genet 19:148-54).

Until this present invention, however, no fly model of a polyglutamine neurodegenerative disorder using the full-length protein as opposed to polyglutamine-containing protein fragments had been described. Because these fragments tend to be more toxic than the full-length proteins and do not elicit the cell-type-specific neurodegeneration characteristic of these diseases (Lin et al., 1999, Neuron 24:499-502), their activities might be related but distinct. The generation by the inventors of transgenic flies expressing full-length ataxin-1 demonstrated that high levels of the wild-type human unexpanded ataxin-1 30Q can produce neurodegenerative phenotypes resembling those produced by lower levels of expanded ataxin-1 82Q.

Citation of a reference herein shall not be construed as an admission that such reference is prior art to the present invention.

### 3. SUMMARY OF THE INVENTION

As described in Section 6 below, the inventors have shown that polyglutamine-induced degeneration in SCA-1 is caused, in part, by impaired protein clearance. The severity of the phenotype correlates strongly with expression levels of mutant ataxin-1 with expanded polyglutamine repeats; sufficiently high levels of wild-type ataxin-1 also result in SCA-1 pathogenesis; additionally, altering the activity of one or more components of the protein folding and proteolytic machineries modifies the SCA-1 phenotype. The ease of detecting subtle modifications in the neurodegenerative phenotype of SCA-1 flies makes them a valuable tool for in vivo pharmacologic screens. The discovery of SCA-1 modifiers involved in GST-mediated cellular detoxification, transcriptional regulation and RNA processing reveal additional pathogenic mechanisms in SCA-1. As described below, these modifiers can be used as therapeutics and diagnostics for SCA-1, and as tools for screening for compounds that inhibit SCA-1. Such compounds will not only be useful for treating SCA-1, but because the underlying mechanisms among many neurodegenerative diseases are similar, the compounds will be useful in the treatment of a variety of neurodegenerative disorders.

The present invention provides a transgenic Drosophila whose somatic and germ cells comprise a transgene operatively linked to a promoter, wherein the transgene encodes normal ataxin-1, and wherein the expression of said transgene in the nervous system results in said Drosophila having a predisposition to progressive neural degeneration. In certain embodiments, the transgene encodes ataxin-1 comprising a polyglutamine repeat having 6-19 glutamine residues. In other embodiments, the transgene encodes ataxin-1 comprising a polyglutamine repeat having 20-40 glutamine residues and 1-4 histidine residues.

The present invention further provides transgenic Drosophila whose somatic and germ cells comprise a transgene operatively linked to a promoter, wherein the transgene encodes ataxin-1 with expanded polyglutamine repeats, and wherein the expression of said transgene in the nervous system results in progressive neural degeneration. In certain embodiments of the invention, the transgene encodes ataxin-1 comprising a polyglutamine repeat having 39-82 glutamine residues. In a preferred mode of the embodiment, the transgene is ataxin-1 82Q.

In preferred embodiments of the invention, the ataxin-1 trangene in the transgenic Drosophila of the invention is operatively linked to a heterologous promoter. In one embodiment, the transgene is temporally regulated by the heterologous promoter. In another embodiment, the transgene is spatially regulated by the heterologous promoter. In a specific embodiment of the invention, the heterologous promoter is a heat shock promoter. In a preferred mode of the embodiment, the heat shock promoter is derived from the hsp70 or hsp83 genes. In other specific embodiments, the ataxin-1 transgene is operatively linked to a Gal4 Upstream Activating Sequence (“UAS”). Optionally, the transgenic Drosophila comprising an ataxin-1 transgene further comprise a GAL4 gene. In a preferred embodiment, the GAL4 gene is linked to a tissue specific promoter. In a preferred mode of the embodiment, the tissue specific promoter is derived from the sevenless, eyeless, or glass genes. In another preferred mode of the embodiment, the tissue specific promoter is derived from the dpp, vestigal, or apterous genes. In yet other embodiments, the heterologous promoter comprises a tetracycline-controlled transcriptional activator (tTA) responsive regulatory element. Optionally, the transgenic Drosophila comprising an ataxin-1 transgene further comprise a tTA gene.

The present invention further provides methods of screening for a molecule having activity against a neurodegenerative disorder, comprising (a) contacting a first transgenic Drosophila which expresses ataxin-1 with expanded polyglutamine repeats in its central nervous system with said molecule; and (b) determining whether the progressive neuronal degeneration in said transgenic Drosophila is less severe than the progressive neuronal degeneration of a second Drosophila which expresses the ataxin-1 with expanded polyglutamine repeats in its central nervous system but wherein said second Drosophila was not contacted with said molecule; wherein a reduction in the progressive neuronal degeneration of the first Drosophila relative to a the second Drosophila is indicative that the molecule has activity against the neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a neural degeneration phenotype.

The present invention further provides methods of screening for a molecule having activity against a neurodegenerative disorder, comprising (a) contacting a first transgenic Drosophila larva which expresses ataxin-1 with expanded polyglutamine repeats in its eye imaginal disc with said molecule, which expression results in a rough eye phenotype; and (b) determining whether the rough eye phenotype in a first adult Drosophila resulting from said first larva is less severe than the rough eye phenotype of a second adult Drosophila resulting from a second larva which expresses the ataxin-1 with expanded polyglutamine repeats in its eye imaginal disc but wherein said second larva was not contacted with said molecule; wherein a reduction in the rough eye phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. As an alternative to Drosophila that express ataxin-1 with expanded polyglutamine repeats, the screen can be accomplished with Drosophila that express sufficient levels of normal ataxin-1 to promote a rough eye phenotype. In a preferred embodiment, the methods screen for molecules with activity against SCA-1.

The present invention further provides methods of screening for a molecule having activity against a neurodegenerative disorder, comprising (a) contacting a first transgenic Drosophila larva which expresses ataxin-1 with expanded polyglutamine repeats in its nervous system with said molecule, which expression results in a locomotor dysfunction and/or a reduced life span; and (b) determining whether the locomotor dysfunction and/or reduced life span in a first adult Drosophila resulting from said first larva is less severe than the locomotor dysfunction and/or reduced life span of a second adult Drosophila resulting from a second larva which expresses the ataxin-1 with expanded polyglutamine repeats in its eye imaginal disc but wherein said second larva was not contacted with said molecule; wherein a reduction in the locomotor dysfunction and/or reduced life span phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a locomotor dysfunction and/or reduced life span phenotype. Further, the ataxin-1 misexpression may be accomplished at the pupal and/or adult stages of development instead of, or in addition to, misexpression at the larval stages.

The present invention further provides methods of screening for a molecule having activity against a neurodegenerative disorder, comprising (a) contacting a first transgenic Drosophila which (i) expresses ataxin-1 with expanded polyglutamine repeats in its central nervous system and (ii) has a gain of function mutation in or misexpresses a SCA-1 suppressor gene with said molecule; and (b) determining whether the progressive neuronal degeneration in said transgenic Drosophila is less severe than the progressive neuronal degeneration of a second Drosophila which expresses the ataxin-1 with expanded polyglutamine repeats in its central nervous system and has a gain of function mutation in or misexpresses a SCA-1 suppressor gene but wherein said second Drosophila was not contacted with said molecule; wherein a reduction in the progressive neuronal degeneration of the first Drosophila relative to a the second Drosophila is indicative that the molecule has activity against the neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a neural degeneration phenotype.

The present invention further provides methods of screening for a molecule having activity against a neurodegenerative disorder, comprising (a) contacting a first transgenic Drosophila larva which (i) expresses ataxin-1 with expanded polyglutamine repeats in its eye imaginal disc and (ii) has a gain of function mutation in or misexpresses a SCA-1 suppressor gene with said molecule, which expression results in a rough eye phenotype; and (b) determining whether the rough eye phenotype in a first adult Drosophila resulting from said first larva is less severe than the rough eye phenotype of a second adult Drosophila resulting from a second larva which expresses the ataxin-1 with expanded polyglutamine repeats in its eye imaginal disc and has a gain of function mutation in or misexpresses a SCA-1 suppressor gene but wherein said second larva was not contacted with said molecule; wherein a reduction in the rough eye phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. As an alternative to Drosophila that express ataxin-1 with expanded polyglutamine repeats, the screen can be accomplished with Drosophila that express sufficient levels of normal ataxin-1 to promote a rough eye phenotype. In a preferred embodiment, the methods screen for molecules with activity against SCA-1.

The present invention further provides methods of screening for a molecule having activity against a neurodegenerative disorder, comprising (a) contacting a first transgenic Drosophila larva which (i) expresses ataxin-1 with expanded polyglutamine repeats in its nervous system and (ii) has a gain of function mutation in or misexpresses a SCA-1 suppressor gene with said molecule, which expression results in a locomotor dysfunction and/or a reduced life span; and (b) determining whether the locomotor dysfunction and/or reduced life span in a first adult Drosophila resulting from said first larva is less severe than the locomotor dysfunction and/or reduced life span of a second adult Drosophila resulting from a second larva which expresses the ataxin-1 with expanded polyglutamine repeats in its eye imaginal disc and has a gain of function mutation in or misexpresses a SCA-1 suppressor gene but wherein said second larva was not contacted with said molecule; wherein a reduction in the locomotor dysfunction and/or reduced life span phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a locomotor dysfunction and/or reduced life span phenotype. Further, the ataxin-1 misexpression may be accomplished at the pupal and/or adult stages of development instead of, or in addition to, misexpression at the larval stages.

The present invention further provides methods of screening for a molecule having activity against a neurodegenerative disorder, comprising (a) contacting a first transgenic Drosophila which (i) expresses ataxin-1 with expanded polyglutamine repeats in its central nervous system and (ii) has a loss of function mutation in a SCA-1 enhancer gene with said molecule; and (b) determining whether the progressive neuronal degeneration in said transgenic Drosophila is less severe than the progressive neuronal degeneration of a second Drosophila which expresses the ataxin-1 with expanded polyglutamine repeats in its central nervous system and has a loss of function mutation in a SCA-1 enhancer gene but wherein said second Drosophila was not contacted with said molecule; wherein a reduction in the progressive neuronal degeneration of the first Drosophila relative to a the second Drosophila is indicative that the molecule has activity against the neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a neural degeneration phenotype.

The present invention further provides methods of screening for a molecule having activity against a neurodegenerative disorder, comprising (a) contacting a first transgenic Drosophila larva which (i) expresses ataxin-1 with expanded polyglutamine repeats in its eye imaginal disc and (ii) has a loss of function mutation in a SCA-1 enhancer gene with said molecule, which expression results in a rough eye phenotype; and (b) determining whether the rough eye phenotype in a first adult Drosophila resulting from said first larva is less severe than the rough eye phenotype of a second adult Drosophila resulting from a second larva which expresses the ataxin-1 with expanded polyglutamine repeats in its eye imaginal disc and has a loss of function mutation in a SCA-1 enhancer gene but wherein said second larva was not contacted with said molecule; wherein a reduction in the rough eye phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. As an alternative to Drosophila that express ataxin-1 with expanded polyglutamine repeats, the screen can be accomplished with Drosophila that express sufficient levels of normal ataxin-1 to promote a rough eye phenotype. In a preferred embodiment, the methods screen for molecules with activity against SCA-1.

The present invention further provides methods of screening for a molecule having activity against a neurodegenerative disorder, comprising (a) contacting a first transgenic Drosophila larva which (i) expresses ataxin-1 with expanded polyglutamine repeats in its nervous system and (ii) has a loss of function mutation in a SCA-1 enhancer gene with said molecule, which expression results in a locomotor dysfunction and/or a reduced life span; and (b) determining whether the locomotor dysfunction and/or reduced life span in a first adult Drosophila resulting from said first larva is less severe than the locomotor dysfunction and/or reduced life span of a second adult Drosophila resulting from a second larva which expresses the ataxin-1 with expanded polyglutamine repeats in its eye imaginal disc and has a loss of function mutation in a SCA-1 enhancer gene but wherein said second larva was not contacted with said molecule; wherein a reduction in the locomotor dysfunction and/or reduced life span phenotype of the first adult Drosophila relative to a the second adult Drosophila is indicative that the molecule has activity against a neurodegenerative disorder. In one embodiment, the ataxin-1 comprises a polyglutamine repeat having 39-82 glutamine residues. In a preferred mode of the embodiment, the ataxin-1 with expanded polyglutamine repeats is ataxin-1 82Q. In a preferred embodiment, the methods screen for molecules with activity against SCA-1. As an alternative to Drosophila that express ataxin-1 with expanded polyglutamine repeats, the screen can be accomplished with Drosophila that express sufficient levels of normal ataxin-1 to promote a locomotor dysfunction and/or reduced life span phenotype. Further, the ataxin-1 misexpression may be accomplished at the pupal and/or adult stages of development instead of, or in addition to, misexpression at the larval stages.

As described in Section 5.14.6 below, the methods described above where a molecule, preferably a candidate drug, is tested for its effects on Drosophila animals that both misexpress ataxin-1 and harbor a mutation in or misexpress a SCA-1 modifier gene are particularly useful when comparing the effect of the molecule on Drosphila that misexpress ataxin-1 but do not harbor a mutation in or misexpress the SCA-1 modifier gene. Specifically, such comparative methods are useful to identify the direct target of the candidate drug. Additionally, the screening methods that employ Drosphila that both misexpress ataxin-1 and harbor a mutation in or misexpress a SCA-1 modifier gene can be done in parallel with screening methods that employ Drosophila that misexpress ataxin-1 but do not harbor a mutation in or misexpress the SCA-1 modifier gene to identify drugs that specifically target the SCA-1 modifier gene.

The present invention further provides methods of screening for a molecule having activity against a neurodegenerative disorder, comprising (a) contacting a first Drosophila which has a loss of function mutation in a SCA-1 enhancer gene with said molecule, thereby producing a loss of function phenotype of the SCA-1 enhancer gene; and (b) determining whether the loss of function phenotype in said Drosophila is less severe than the loss of function phenotype of a second Drosophila which has the loss of function mutation in a SCA-1 enhancer gene but wherein said second Drosophila was not contacted with said molecule; wherein an amelioration in the loss of function phenotype of the first Drosophila relative to a the second Drosophila is indicative that the molecule has activity against the neurodegenerative disorder. In a preferred embodiment, the methods screen for molecules with activity against SCA-1. As an alternative to conducting the screen with animals with a loss of function mutation in a SCA-1 enhancer gene, the screen can be conducted with animals that misexpress or have a gain of function mutation in a SCA-1 suppressor gene.

The present invention further provides methods of screening for a molecule having activity against a neurodegenerative disorder, comprising (a) contacting a first Drosophila larva which has a loss of function mutation in a SCA-1 enhancer gene with said molecule, thereby producing a loss of function phenotype of the SCA-1 enhancer gene; and (b) determining whether the loss of function phenotype in a first adult Drosophila resulting from said first larva is less severe than the loss of function phenotype of a second adult Drosophila resulting from a second larva which has a loss of function mutation in the SCA-1 enhancer gene but wherein said second larva was not contacted with said molecule; wherein an amelioration of the loss of function phenotype of the first adult Drosophila relative to a the second adult Drosophila is indicative that the molecule has activity against a neurodegenerative disorder. In a preferred embodiment, the methods screen for molecules with activity against SCA-1. As an alternative to conducting the screen with animals with a loss of function mutation in a SCA-1 enhancer gene, the screen can be conducted with animals that misexpress or have a gain of function mutation in a SCA-1 suppressor gene.

The present invention further provides methods of screening for a molecule having activity against a neurodegenerative disorder, comprising (a) contacting a first Drosophila which has a loss of function mutation in a SCA-1 suppressor gene with said molecule, thereby producing a loss of function phenotype of the SCA-1 suppressor gene; and (b) determining whether the loss of function phenotype in said Drosophila is more severe than the loss of function phenotype of a second Drosophila which has the loss of function mutation in a SCA-1 suppressor gene but wherein said second Drosophila was not contacted with said molecule; wherein an exacerbation in the loss of function phenotype in the first Drosophila relative to a the second Drosophila is indicative that the molecule has activity against the neurodegenerative disorder. In a preferred embodiment, the methods screen for molecules with activity against SCA-1. As an alternative to conducting the screen with animals with a loss of function mutation in a SCA-1 suppressor gene, the screen can be conducted with animals that misexpress or have a gain of function mutation in a SCA-1 enhancer gene.

The present invention further provides methods of screening for a molecule having activity against a neurodegenerative disorder, comprising (a) contacting a first Drosophila larva which has a loss of function mutation in a SCA-1 suppressor gene with said molecule, thereby producing a loss of function phenotype of the SCA-1 suppressor gene; and (b) determining whether the loss of function phenotype in a first adult Drosophila resulting from said first larva is more severe than the loss of function phenotype of a second adult Drosophila resulting from a second larva which has a loss of function mutation in the SCA-1 suppressor gene but wherein said second larva was not contacted with said molecule; wherein an exacerbation of the loss of function phenotype in the first adult Drosophila relative to a the second adult Drosophila is indicative that the molecule has activity against a neurodegenerative disorder. In a preferred embodiment, the methods screen for molecules with activity against SCA-1. As an alternative to conducting the screen with animals with a loss of function mutation in a SCA-1 suppressor gene, the screen can be conducted with animals that misexpress or have a gain of function mutation in a SCA-1 enhancer gene.

The present invention further provides methods of screening for a molecule with activity against a vertebrate disease, comprising (a) contacting a first transgenic Drosophila larva which expresses a vertebrate disease gene associated with said vertebrate disease in its central nervous system with said molecule, said expression of said vertebrate disease gene resulting in a behavioral disorder; and (b) determining whether the behavioral disorder in a first adult Drosophila resulting from said first larva is less severe than the behavioral disorder of a second adult Drosophila resulting from a second larva which expresses said vertebrate disease gene in its central nervous system but wherein said second larva was not contacted with said molecule; wherein a reduction in severity of the behavioral disorder of the first adult Drosophila relative to a the second adult Drosophila is indicative that the molecule has activity against the vertebrate disease. In one embodiment, the vertebrate disease is a neurodegenerative disorder, including but not limited to a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder. In a preferred mode of the embodiment, the neurodegenerative disorder is a polyglutamine disease, including but not limited to SCA-1, SCA-2, SCA-6, SCA-7, MJD, HD, SBMA, or DRPLA. In embodiment of the behavioral screening assays, the vertebrate disease gene encodes ataxin-1 with expanded polyglutamine repeats. In other embodiments, the vertebrate disease gene encodes tau, synuclein, prion protein, huntingtin, or ataxin-3.

In another embodiment of the behavioral screening assays, the vertebrate disease is a proliferative disorder such as cancer. In specific modes of the embodiment, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, or heavy chain disease.

In yet another embodiment of the behavioral screening assays, the vertebrate disease is a skeletal muscle disorder, such as a muscular dystrophy, a motor neuron disease, or a myopathy.

Optionally, the transgenic Drosophila used in the behavioral screening assays of the invention, comprising a vertebrate disease gene under the control of a UAS element, further comprise a GAL4 gene. In a preferred embodiment, the GAL4 gene is linked to a tissue specific promoter. In a preferred mode of the embodiment, the tissue specific promoter is derived from the elav, Appl, or nirvana genes.

The present invention yet further provides methods of identifying of a modifier gene of SCA-1 comprising (a) generating a cross between a transgenic Drosophila whose somatic and germ cells comprise a transgene operatively linked to a promoter, wherein the transgene encodes ataxin-1 with expanded polyglutamine repeats, wherein the expression of said transgene in the nervous system results in progressive neural degeneration; and a second Drosophila suspected of having one or more mutations in its germ cells, to produce progeny; (b) determining whether the progeny of said cross have a modified phenotype associated with the ataxin-1 transgene, wherein a modification of the phenotype associated with the ataxin-1 transgene is indicative that the second Drosophila has a mutation in a modifier gene of SCA-1; and (c) identifying the gene responsible for the modified phenotype associated with associated with the ataxin-1 transgene; wherein the gene identified in step (c) is a modifier gene of SCA-1. The methods optionally further entail the step (d) of identifying a mammalian homolog of said modifier gene of SCA-1. In one embodiment, said modification of the phenotype associated with the ataxin-1 transgene is an enhancement of the phenotype, said mutation responsible for the enhancement of the phenotype is a loss of function mutation, and said modifier gene of SCA-1 is an enhancer gene of SCA-1. In another embodiment, said modification of the phenotype associated with the ataxin-1 transgene is a suppression of the phenotype, said mutation responsible for the for the suppression of the phenotype is a gain of function mutation, and said modifier gene of SCA-1 is an enhancer gene of SCA-1. In another embodiment, said modification of the phenotype associated with the ataxin-1 transgene is a suppression of the phenotype, said mutation responsible for the for the suppression of the phenotype is a loss of function mutation, and said modifier gene of SCA-1 is a suppressor gene of SCA-1. In yet another embodiment, said modification of the phenotype associated with the ataxin-1 transgene is an enhancement of the phenotype, said mutation responsible for the for the enhancement of the phenotype is a gain of function mutation, and said modifier gene of SCA-1 is a suppressor gene of SCA-1.

The present invention further provides methods of identifying a modifier gene of SCA-1, comprising (a) crossing a transgenic Drosophila whose somatic and germ cells comprise a transgene operatively linked to a promoter, wherein the transgene encodes ataxin-1 with expanded polyglutamine repeats, wherein the expression of said transgene in the nervous system results in progressive neural degeneration, to a mutagenized Drosophila, to produce progeny; (b) determining whether the progeny of the cross of step (a) have a modified phenotype associated with the ataxin-1 transgene, wherein a modification of the phenotype associated with the ataxin-1 transgene is indicative that the mutagenized Drosophila has a mutation in a modifier gene of SCA-1; and (c) identifying the gene responsible for the modified phenotype associated with associated with the ataxin-1 transgene; wherein the gene identified in step (c) is a modifier gene of SCA-1. The methods optionally further entail the step (d) of identifying a mammalian homolog of said modifier gene of SCA-1. In one embodiment, said modification of the phenotype associated with the ataxin-1 transgene is an enhancement of the phenotype, said mutation responsible for the enhancement of the phenotype is a loss of function mutation, and said modifier gene of SCA-1 is an enhancer gene of SCA-1. In another embodiment, said modification of the phenotype associated with the ataxin-1 transgene is a suppression of the phenotype, said mutation responsible for the for the suppression of the phenotype is a gain of function mutation, and said modifier gene of SCA-1 is an enhancer gene of SCA-1. In another embodiment, said modification of the phenotype associated with the ataxin-1 transgene is a suppression of the phenotype, said mutation responsible for the for the suppression of the phenotype is a loss of function mutation, and said modifier gene of SCA-1 is a suppressor gene of SCA-1. In yet another embodiment, said modification of the phenotype associated with the ataxin-1 transgene is an enhancement of the phenotype, said mutation responsible for the for the enhancement of the phenotype is a gain of function mutation, and said modifier gene of SCA-1 is a suppressor gene of SCA-1.

The present invention further provides methods of treating or preventing a neurodegenerative disorder, comprising (a) administering to a subject in need of such treatment an antagonist of a suppressor gene of SCA-1. In yet other aspects, the present invention further provides methods of treating or preventing SCA-1, comprising (a) identifying a suppressor gene of SCA-1 according to the methods described herein; and (b) administering to a subject in need of such treatment an antagonist of said suppressor gene of SCA-1. In one embodiment, the antagonist is an antisense RNA or ribozyme. In another embodiment, the antagonist is an antibody, peptide, or small molecule.

The present invention further provides methods of treating or preventing a neurodegenerative disorder, comprising (a) administering to a subject in need of such treatment an agonist of an enhancer gene of SCA-1. In yet other aspects, the present invention further provides methods of treating or preventing a neurodegenerative disorder, comprising (a) identifying an enhancer gene of SCA-1 according to the methods described herein; and (b) administering to a subject in need of such treatment an agonist of said enhancer gene of SCA-1. In one embodiment, the agonist is gene therapy vector encoding the enhancer gene of SCA-1. In one mode of the embodiment, the gene therapy vector is an adenovirus, adeno-associated virus, retrovirus, or liposome.

The present invention further provides methods of screening for a molecule with activity against a neurodegenerative disorder, comprising (a) screening for a molecule that agonizes said enhancer gene of SCA-1; wherein a molecule that agonizes said enhancer gene of SCA-1 is molecule with activity against the a neurodegenerative disorder. In yet other aspects, the present invention further provides methods of screening for a molecule with activity against a neurodegenerative disorder, comprising (a) identifying an enhancer gene of SCA-1 according to the methods described herein; and (b) screening for a molecule that agonizes said enhancer gene of SCA-1; wherein a molecule that agonizes said enhancer gene of SCA-1 is molecule with activity against the neurodegenerative disorder.

The present invention further provides methods of screening for a molecule with activity against a neurodegenerative disorder, comprising (a) screening for a molecule that antagonizes a suppressor gene of SCA-1; wherein a molecule that antagonizes said suppressor gene of SCA-1 is molecule with activity against the neurodegenerative disorder. In yet other aspects, the present invention further provides methods of screening for a molecule with activity against a neurodegenerative disorder, comprising (a) identifying a suppressor gene of SCA-1 according to the methods described herein; and (b) screening for a molecule that antagonizes said suppressor gene of SCA-1; wherein a molecule that antagonizes said suppressor gene of SCA-1 is molecule with activity against the neurodegenerative disorder.

The present invention further provides methods of diagnosing a predisposition to SCA-1 in an individual, comprising (a) measuring the expression level of normal ataxin-1 in a sample from said individual; and (b) determining whether said expression level is higher than normal expression of ataxin-1, wherein a higher expression level of ataxin-1 is indicative of a predisposition to SCA-1. In one embodiment, the higher level of ataxin-1 indicative of a predisposition to SCA-1 is at least 25% more than normal expression of ataxin-1. In another embodiment, the higher level of ataxin-1 indicative of a predisposition to SCA-1 is at least 50% more than normal expression of ataxin-1. In yet another embodiment, the higher level of ataxin-1 indicative of a predisposition to SCA-1 is at least 75% more than normal expression of ataxin-1. In yet another embodiment, the higher level of ataxin-1 indicative of a predisposition to SCA-1 is at least twofold the normal expression of ataxin-1. Measuring ataxin-1 expression can be accomplished by measuring ataxin-1 RNA or ataxin-1 protein.

The present invention further provides pharmaceutical compositions for the treatment or prevention of a neurodegenerative disorder, comprising (a) a glutathione-S-transferase agonist and (b) a pharmaceutically acceptable carrier. In one embodiment, the glutathione-S-transferase agonist is a nucleic acid encoding a glutathione-S-transferase protein. In one mode of the embodiment, the glutathione-S-transferase protein is a theta class glutathione-S-transferase protein. In another mode of the embodiment, the glutathione-S-transferase protein is a sigma class glutathione-S-transferase protein.

The present invention further provides methods of treating or preventing a neurodegenerative disorder, comprising administering to an individual in the need of such treatment or prevention a glutathione-S-transferase agonist in an amount effective for the treatment or prevention of the neurodegenerative disorder. In one embodiment, the glutathione-S-transferase agonist is a nucleic acid encoding a glutathione-S-transferase protein. In one mode of the embodiment, the glutathione-S-transferase protein is a theta class glutathione-S-transferase protein. In another mode of the embodiment, the glutathione-S-transferase protein is a sigma class glutathione-S-transferase protein.

The present invention further provides pharmaceutical compositions for the treatment or prevention of a neurodegenerative disorder, comprising (a) a Sin3A agonist and (b) a pharmaceutically acceptable carrier. In one embodiment, the Sin3A agonist is a nucleic acid encoding a Sin3A protein.

The present invention further provides methods of treating or preventing a neurodegenerative disorder, comprising administering to an individual in the need of such treatment or prevention a Sin3A agonist in an amount effective for the treatment or prevention of the neurodegenerative disorder. In one embodiment, the a Sin3A agonist is a nucleic acid encoding a Sin3A protein.

The present invention further provides pharmaceutical compositions for the treatment or prevention of a neurodegenerative disorder, comprising (a) a CtBP agonist and (b) a pharmaceutically acceptable carrier. In one embodiment, the CtBP agonist is a nucleic acid encoding a CtBP protein.

The present invention further provides methods of treating or preventing a neurodegenerative disorder, comprising administering to an individual in the need of such treatment or prevention a CtBP agonist in an amount effective for the treatment or prevention of the neurodegenerative disorder. In one embodiment, the a CtBP agonist is a nucleic acid encoding a CTBP protein.

The present invention further provides pharmaceutical compositions for the treatment or prevention of a neurodegenerative disorder, comprising (a) a Trap240 agonist and (b) a pharmaceutically acceptable carrier. In one embodiment, the Trap240 agonist is a nucleic acid encoding a Trap240 protein.

The present invention further provides methods of treating or preventing a neurodegenerative disorder, comprising administering to an individual in the need of such treatment or prevention a Trap240 agonist in an amount effective to treat of prevent the neurodegenerative disorder. In one embodiment, the Trap240 agonist is a nucleic acid encoding a Trap240 protein.

The present invention further provides pharmaceutical compositions for the treatment or prevention of a neurodegenerative disorder, comprising (a) a Ubi63E agonist and (b) a pharmaceutically acceptable carrier. In one embodiment, the Ubi63E agonist is a nucleic acid encoding a Ubi63E protein.

The present invention further provides methods of treating or preventing a neurodegenerative disorder, comprising administering to an individual in the need of such treatment or prevention a Ubi63E agonist in an amount effective for the treatment or prevention of the neurodegenerative disorder. In one embodiment, the Ubi63E agonist is a nucleic acid encoding a Ubi63E protein.

The present invention further provides pharmaceutical compositions for the treatment or prevention of a neurodegenerative disorder, comprising (a) a UbcD1 agonist and (b) a pharmaceutically acceptable carrier. In one embodiment, the UbcD1 agonist is a nucleic acid encoding a UbcD1 protein.

The present invention further provides methods of treating or preventing a neurodegenerative disorder, comprising administering to an individual in the need of such treatment or prevention a UbcD1 agonist in an amount effective for the treatment or prevention of the neurodegenerative disorder. In one embodiment, the UbcD1 agonist is a nucleic acid encoding a UbcD1 protein.

The present invention further provides pharmaceutical compositions for the treatment or prevention of a neurodegenerative disorder, comprising (a) a nup44A agonist and (b) a pharmaceutically acceptable carrier. In one embodiment, the nup44A agonist is a nucleic acid encoding a nup44A protein.

The present invention further provides methods of treating or preventing a neurodegenerative disorder, comprising administering to an individual in the need of such treatment or prevention a nup44A agonist in an amount effective for the treatment or prevention of the neurodegenerative disorder. In one embodiment, the nup44A agonist is a nucleic acid encoding a nup44A protein.

The present invention further provides pharmaceutical compositions for the treatment or prevention of a neurodegenerative disorder, comprising (a) a mub agonist and (b) a pharmaceutically acceptable carrier. In one embodiment, the mub agonist is a nucleic acid encoding a mub protein.

The present invention further provides methods of treating or preventing a neurodegenerative disorder, comprising administering to an individual in the need of such treatment or prevention a mub agonist in an amount effective for the treatment or prevention of the neurodegenerative disorder. In one embodiment, the mub agonist is a nucleic acid encoding a mub protein.

The present invention further provides pharmaceutical compositions for the treatment or prevention of a neurodegenerative disorder, comprising (a) a cpo agonist and (b) a pharmaceutically acceptable carrier. In one embodiment, the cpo agonist is a nucleic acid encoding a cpo protein.

The present invention further provides methods of treating or preventing a neurodegenerative disorder, comprising administering to an individual in the need of such treatment or prevention a cpo agonist in an amount effective for the treatment or prevention of the neurodegenerative disorder. In one embodiment, the cpo agonist is a nucleic acid encoding a cpo protein.

The present invention further provides pharmaceutical compositions for the treatment or prevention of a neurodegenerative disorder, comprising (a) a Rpd3 agonist and (b) a pharmaceutically acceptable carrier. In one embodiment, the Rpd3 agonist is a nucleic acid encoding a Rpd3 protein.

The present invention further provides methods of treating or preventing a neurodegenerative disorder, comprising administering to an individual in the need of such treatment or prevention a Rpd3 agonist in an amount effective for the treatment or prevention of the neurodegenerative disorder. In one embodiment, the Rpd3 agonist is a nucleic acid encoding a Rpd3 protein.

The present invention further provides pharmaceutical compositions for the treatment or prevention of a neurodegenerative disorder, comprising (a) a tara agonist and (b) a pharmaceutically acceptable carrier. In one embodiment, the tara agonist is a nucleic acid encoding a tara protein.

The present invention further provides methods of treating or preventing a neurodegenerative disorder, comprising administering to an individual in the need of such treatment or prevention a tara agonist in an amount effective for the treatment or prevention of the neurodegenerative disorder. In one embodiment, the tara agonist is a nucleic acid encoding a tara protein.

The present invention further provides pharmaceutical compositions for the treatment or prevention of a neurodegenerative disorder, comprising (a) a hsr-ω agonist and (b) a pharmaceutically acceptable carrier. In one embodiment, the tara agonist is a nucleic acid encoding a hsr-ω protein.

The present invention further provides methods of treating or preventing a neurodegenerative disorder, comprising administering to an individual in the need of such treatment or prevention a hsr-ω agonist in an amount effective for the treatment or prevention of the neurodegenerative disorder. In one embodiment, the hsr-ω agonist is a nucleic acid encoding a hsr-ω protein.

The present invention further provides pharmaceutical compositions for the treatment or prevention of a neurodegenerative disorder, comprising (a) a trithorax agonist and (b) a pharmaceutically acceptable carrier. In one embodiment, the trithorax agonist is a nucleic acid encoding a trithorax protein.

The present invention further provides methods of treating or preventing a neurodegenerative disorder, comprising administering to an individual in the need of such treatment or prevention a trithorax agonist in an amount effective to treat of prevent the neurodegenerative disorder. In one embodiment, the trithorax agonist is a nucleic acid encoding a trithorax protein.

The present invention further provides pharmaceutical compositions for the treatment or prevention of a neurodegenerative disorder, comprising (a) a KH-domain protein agonist and (b) a pharmaceutically acceptable carrier. In one embodiment, the KH-domain protein agonist is a nucleic acid encoding a KH-domain protein.

The present invention further provides methods of treating or preventing a neurodegenerative disorder, comprising administering to an individual in the need of such treatment or prevention a KH-domain protein agonist in an amount effective to treat of prevent the neurodegenerative disorder. In one embodiment, the KH-domain protein agonist is a nucleic acid encoding a KH-domain protein.

The present invention further provides pharmaceutical compositions for the treatment or prevention of a neurodegenerative disorder, comprising (a) a modulator of a gene product of dUbc-E2H, DnaJ-1 64EF, pum, dYT521-B, dSir2, CG6785, Dsp1, HmgD, CG10934, CG3445, CG6783, Xnp, CG1910, CG5261, CG4834, CG18445, Lilliputian/CG8817, CG8062, Act5C/CG4027, CG8240, CG14438, CG9650, CG7233, pipsqueak, elbow B, CG10882, CG14757, CG8204, CG12846, Pk61C, Rac2, CG14959, CG5166, CG14363, boule, CG12084, CG7518, vibrator/CG5269, CG9246, CG11171, pKa-C1, KEK1, CG6301, lesswright, spen, mastermind, CG11278/Syx13, CG6767, CG5891, CG10733, jumu, pebble, shank, hsp83, tacc, guftagu, CG9988, ariadne-2, or Gbp, and (b) a pharmaceutically acceptable carrier. In one embodiment, the modulator is an agonist of DnaJ-1 64EF Dsp1, CG10934, CG3445, Xnp, CG1910, CG5261, CG8062, Act5C/CG4027, CG8240, CG9650, CG7233, pipsqueak, elbow B, CG14757, CG8204, CG12846, Rac2, CG5166, CG14363, boule, CG12084, CG9246, CG11171, pKa-C1, CG6301, guftagu, ariadne-2, or Gbp. In another embodiment, the modulator is an antagonist of dUbc-E2H, pum, dYT521-B, dSir2, CG6785, HmgD, CG6783, CG4834, CG18445, Lilliputian/CG8817, CG10882, Pk61C, CG14959, CG7518, vibrator/CG5269, KEK1, lesswright, spen, mastermind, CG11278/Syx13, CG6767, CG5891, CG10733, jumu, pebble, shank, hsp83, tacc, or CG9988.

The present invention further provides methods of treating or preventing a neurodegenerative disorder, comprising administering to an individual in the need of such treatment or prevention a modulator of a gene or its gene product selected from the group consisting of dUbc-E2H, DnaJ-1 64EF, pum, dYT521-B, dSir2, CG6785, Dsp1, HmgD, CG10934, CG3445, CG6783, Xnp, CG1910, CG5261, CG4834, CG18445, Lilliputian/CG8817, CG8062, Act5C/CG4027, CG8240, CG14438, CG9650, CG7233, pipsqueak, elbow B, CG10882, CG14757, CG8204, CG12846, Pk61C, Rac2, CG14959, CG5166, CG14363, boule, CG12084, CG7518, vibrator/CG5269, CG9246, CG11171, pKa-C1, KEK1, CG6301, lesswright, spen, mastermind, CG11278/Syx13, CG6767, CG5891, CG10733, jumu, pebble, shank, hsp83, tacc, guftagu, CG9988, ariadne-2, and Gbp, in an amount effective to treat of prevent the neurodegenerative disorder. In a preferred embodiment, the modulator is an agonist of DnaJ-1 64EF Dsp1, CG10934, CG3445, Xnp, CG1910, CG5261, CG8062, Act5C/CG4027, CG8240, CG9650, CG7233, pipsqueak, elbow B, CG14757, CG8204, CG12846, Rac2, CG5166, CG14363, boule, CG12084, CG9246, CG11171, pKa-C1, CG6301, guftagu, ariadne-2, or Gbp. In another preferred embodiment, the modulator is an antagonist of dUbc-E2H, pum, dYT521-B, dSir2, CG6785, HmgD, CG6783, CG4834, CG18445, Lilliputian/CG8817, CG10882, Pk61C, CG14959, CG7518, vibrator/CG5269, KEK1, lesswright, spen, mastermind, CG11278/Syx13, CG6767, CG5891, CG10733, jumu, pebble, shank, hsp83, tacc, or CG9988.

The methods and compositions of the present invention are useful for the treatment and prevention of polyglutamine diseases, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, taupathies, spongiform encephalopathies, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorders, hypertension, neuropsychiatric disorders, schizophrenia, and schizoaffective disorders, and identifying therapeutics for the foregoing diseases. In a particular aspect of the invention, the methods and compositions of the invention are useful for the treatment or prevention of polyglutamine diseases, including but not limited to spinocerebellar ataxia (SCA)-1, SCA-2, SCA-6, SCA-7, Machado-Joseph disease (MJD), Huntington Disease (HD), spinobulbar muscular atrophy (SBMA), and dentatorubropallidolusyan atrophy (DRPLA), as well as for identifying therapeutics for the foregoing diseases. In a particularly preferred embodiment, the methods and compositions of the invention are used to treat or prevent SCA-1, and to identify therapeutics of SCA-1.

3.1. Definitions

Ataxin-1 gene: A nucleic acid encoding a normal ataxin-1 protein, an ataxin-1 protein with expanded polyglutamine repeats, or a fragment or derivative thereof. The ataxin-1 gene is optionally operatively linked to a regulatory element, a 5′ untranslated region, a 3′ untranslated region, or a combination of the foregoing.

Misexpression of a gene: as used herein, misexpression of a gene of interest (including but not limited to ataxin-1 and SCA-1 modifiers) encompasses overexpression of the gene (i.e., expression at higher than normal levels), expression of the gene in a temporal pattern different from that in which the gene is normally expressed, expression of the gene in a spatial pattern different from that in which the gene is normally expressed, or a combination of the foregoing.

SCA-1 phenotype: a phenotype resulting from the misexpression of a normal ataxin-1 protein or the expression of an ataxin-1 protein with expanded polyglutamine repeats. A SCA-1 phenotype in Drosophila includes a rough eye, nuclear inclusions, neuronal degeneration, locomotor dysfunction, and/or reduced lifespan, depending on the spatial and temporal parameters the ataxin misexpression.

SCA-1 modifier gene: a Drosophila gene whose misexpression or mutation results in an enhancement or suppression of the SCA-1 phenotype, or a vertebrate homolog of a Drosophila gene whose misexpression or mutation results in an enhancement or suppression of the SCA-1 phenotype.

SCA-1 enhancer gene: a gene whose loss of function results in more severe SCA-1 pathogenesis. Alternatively, a SCA-1 enhancer gene is one whose misexpression or gain of function results in less severe SCA-1 pathogenesis. In certain embodiments, a SCA-1 enhancer gene is a gene whose loss of function results in more severe SCA-1 pathogenesis and whose misexpression or gain of function results in less severe SCA-1 pathogenesis.

SCA-1 suppressor gene: a gene whose loss of function results in less severe SCA-1 pathogenesis. Alternatively, a SCA-1 suppressor gene is one whose misexpression or gain of function results in more severe SCA-1 pathogenesis. In certain embodiments, a SCA-1 suppressor gene is a gene whose loss of function results in less severe SCA-1 pathogenesis and whose misexpression or gain of function results in more severe SCA-1 pathogenesis.

### 4. BRIEF DESCRIPTION OF THE FIGURES

FIG. 1A-G: Strong (ataxin-1 82Q) and weak (ataxin-1 30Q) eye phenotypes produced by SCA-1 overexpression.

Human SCA-1 constructs injected in Drosophila are shown on top. Top row: scanning electron microscopy (SEM) eye images of gmr-GAL4/+ control (A), gmr-GAL4/+; UAS: SCA-130Q[F6]I+ (B), and gmr-GAL4/+; UAS.˜SCAJ82Q[M6]I+ (C) transgenic flies. Insets show magnification of ommatidia field. Middle row: sections through the eye retinas of gmr-GAL4/+ control (D), gmr-GAL4/+; SCA-1 30Q[F61/+ (E), and gmr-GAL4/+; SCA-1 82Q[M6]/+ (F) transgenic flies. Note the severe degeneration of the retina (arrow) in 82Q[M6] flies, and the relatively milder phenotype of 30Q[F6] flies. Bottom row: Third instar eye imaginal discs stained with anti-ataxin-1 antiserum. No protein is detected in controls (G). Note the similar expression levels of ataxin-1 30Q[F6] (H) and ataxin˜1 82Q[M6] (I) transgenic lines. Numbers in the box indicate the relative amounts of immunofluorescence in the 82Q[M6] and 30Q[F6] lines (see Methods). Insets are magnifications showing ataxin-1 NI (green) and the nuclear membrane (red) revealed by anti-laminin antibody. All images are from flies raised at 23° C.

FIG. 2A-G: ataxin-1 82Q and relatively high levels of ataxin-1 30Q cause similar phenotypes in Drosophila and mice.

Top row: A and C, SEM images of UAS: SCAJ 30Q[FJ]/+; gmr-GAL4/+ eyes raised at 18° C. and 29° C. respectively. B and D, third-instar eye imaginal discs as in A and C respectively stained with anti-ataxin-1 antiserum. Ataxin-1 expression at 29° C. is roughly 158% when compared to ataxin-1 expression at 18° C. as determined by the amount of immunofluorescence (see Methods). Note the aggravation of the phenotype caused by—58% increase in expression level as estimated by immunofluorescence. Bottom row shows mouse cerebellar sections with stained with antisera against the Purkinje cell-specific protein calbindin. E, wild-type mouse, 51 week old. F, heterozygous SCA-1 30Q[A02] mouse, 52 week old. G, homozygous SCA-1 30Q[A02] mouse, 59 week old. H, heterozygous SCA-1 82Q[B05] mouse, 50 week old. Compare the thickness of the molecular layers (arrows) in wild-type (E) and SCA-1 82Q heterozygous mice (H), and note the relatively milder, but clear mutant phenotype of SCA-1 30Q[A02] homozygous mice (G).

FIG. 3A-C. Expression of ataxin-1 82Q in Drosophila intemeurons causes progressive degeneration.

All panels show apterous-expressing intemeurons in the first (Ti) and second (T2) thoracic segments of the adult CNS. There are two intemeurons per hemisegment that are visualized with the t-GFP reporter gene driven by the ap^{VNC} GAL4 driver. Panel A shows CNS of 45-day old control ap^{VNC} GAL4/+; UAS: τ-GFP/+; UAS: lacZI+ fly. Note the robust axonal projections of interneurons that fasciculate with axons from intemeurons in other segments. B high magnification of panel A showing two T1 and two T2 ventral interneurons and their axonal projections. C and D high magnification images of 1-day (C) and 45-day (D) old ap^{VNC} GAL4/+;UAS: τ-GFP/-i-; UAS: SCA-1 82Q[M6]/+ flies. Ataxin-1 82Q accumulates in NI (red label). Most cell bodies are present at day 1 although label in axonal projections is already weak (these interneurons form several days earlier during metamorphosis). At day 45, fewer cell bodies are visible. See Table 1 for numbers.

FIG. 4A-C. Ataxin-1 in Drosophila forms nuclear inclusions that also accumulate Hsp70, Ubiquitin and components of the proteasome.

A-C salivary gland nuclei of control UAS: SCA-1 82Q[F7]/+ (A), UAS: SCA-1 30Q[FJ]/+; dppGAL4/+ (B), and UAS: SCAJ82Q[F71/+ dppGAL4/+ (C) flies stained with ataxin-1 antisera. Note the NI formed in ataxin-1 30Q[F1] (B), and ataxin-1 82Q[F7] flies (C). D-F NI accumulate Ubiquitin labeled in green. G-H NI accumulate the 19S regulator ATPase subunit 6b of the proteasome. J-L NI accumulate the Hsp/Hsc70 molecular chaperone(s). In all panels ataxin-1 is labeled in red whereas Ubiquitin, the subunit of the proteasome, and Hsp70 are labeled in green. Yellow indicates superimposed green and red labels.

FIG. 5A-F. Reduction of the activity of either molecular chaperones, or a component of the proteasome aggravates the ataxin-1 82Q eye phenotype.

A control showing the eye phenotype of UAS: SCAJ 82Q[F71/+; gmr-GAL4/+ flies raised at 23° C. B eye phenotype of flies as in panel A, but carrying the hsc70-4′″ mutation in heterozygosis. C eye phenotype of flies as in panel A, but also heterozygous for Df(3R)karD1, a small deletion removing a cluster of hsp70 genes (hsp70Ab, hsp70Ba, hsp70Bb, and hsp70Bc). D control UAS: SCAJ 82Q[F7]/+; gmr-GAL4/+ raised at 27.5° C. E eye phenotype of flies as in panel D, but also heterozygous for Pros26-DTS. Panel F shows Pros26-DTS/+ control at 27.5° C.

FIG. 6A-K. Suppressors and enhancers in the protein folding heat-shock response, and ubiquitin-proteolytic pathways.

A control showing the eye phenotype of UAS: SCA-1 82Q[F7]/+; gmr-GAL4/+ flies raised at 23 ° C. Note the enhancement of this phenotype in panels B-E. These panels show flies of the same genotype as control in panel A, with the following modifications: B also heterozygous for P 1666, a mutation in Ubiquitin 63E. C also heterozygous for P 1779, a mutation in the Ubiquitin conjugase UbcDl. D also heterozygous for EP1303, an insertion in the Ubiquitin conjugase dUbcE2H. E also heterozygous for P292, a mutation in the heat-shock response factor hsr-co. F (fresh eye image) and G (SEM eye image) show the eye phenotype of UAS: SCA-1 82Q[F7]/+; gmr-GAL4/+ control flies raised at 27° C. Note the rough eye surface and disorganized ommatidia in G, and the relatively little pigmentation in F. H (fresh eye image) and I (SEM eye image) phenotype of flies as in panels F/G, but also carrying EP41 1 that overexpresses DNA J-1 64F. Note that the eye in I is smoother than the eye in G, and has more organized ommatidia. Also, the eye in H is more pigmented than the eye in F; this increase in pigmentation is not seen with other EP's. J salivary glands of UAS: SCAJ 82Q[F7]/+; dppGAL4/+ flies raised at 23° C. stained with anti ataxin-1 antiserum to reveal the NI. K salivary glands as in panel I, but also overexpressing DNA J-J 64F. Note the more compact and less invasive NI.

FIG. 7A-S. Suppressors and enhancers in novel pathways.

A (fresh eye image) and E (SEM eye image) controls showing the eye phenotype of UAS: SCA-182Q[F7]/+; gmr-GAL4/+ flies raised at 27° C. Note the relatively little pigmentation in A, and the roughness and disorganization of the ommatidia field in E. These phenotypes are partially suppressed in panels B-D and F-H. These panels show flies of the same genotype as controls with the following modifications: B (fresh eye image) and F (SEM eye image) also carrying EP223 1 that overexpresses GstS5F. C (fresh eye image) and G (SEM eye image) also carrying EP2417, overexpressing nup44A. D (fresh eye image) and H (SEM eye image) also carrying EP3623 that overexpresses mub. I control showing the mild eye phenotype of controls (UAS:SCA-1 82Q[F7]/+; gmr-GAL4/+) raised at 23° C. Note the enhancement of this phenotype in panels J-T. Eyes in J-T panels are also less pigmented than control eye in I (not shown). J-T panels show fly eyes with the same genotype as I, with the following modifications: J also heterozygous for EP2231M⁸, an imprecise excision of EP2231. K also heterozygous for P1480, a mutation in Gst2. L also carrying EP3461 that overexpresses pum. M also carrying EP3378 that overexpresses cpo. N also carrying EP3725 that overexpresses dYT52J-B. O also heterozygous for EP866, a mutation in Sin3A. P also heterozygous for EP3672, a mutation in Rpd3. Q also heterozygous for P 1590, a mutation in dCtBP. R also carrying EP2300, that overexpresses dSir2. S also heterozygous for P198, a mutation in pap. T also heterozygous for EP3463, a mutation in tara.

### 5. DETAILED DESCRIPTION OF THE INVENTION

As described herein, the inventors have developed a strategy to analyze spinocerebral ataxia-1 (SCA-1) using a Drosophila model. Using the methods described herein, the inventors have identified novel aspects of the function of ataxin-1, a protein encoded by the gene responsible for SCA-1. Further, genes that modify ataxin-1 activity have been characterized as described herein. The Drosophila model of SCA-1 has proven a very useful tool for probing the function and regulation of the cellular pathways involving SCA-1. Systematic genetic analysis of these pathways in Drosophila can be expected to lead to the discovery of new drug targets, therapeutics (including but not limited to the proteins encoded by the modifier genes) useful in the treatment of SCA-1, as well as SCA-1 diagnostics and prognostics.

The invention is illustrated by way of examples set forth in Section 6 below which disclose, inter alia, the characterization of misexpression of a normal ataxin-1 protein (ataxin-1 30Q) and a mutant ataxin-1 protein (ataxin-1 82Q) which has expanded polyglutamine repeats, both of which produce SCA-1 pathologies in Drosophila. Modifiers of the ataxin-1 phenotypes will provide new drug targets, therapeutics, diagnostics and prognostics of SCA-1.

For clarity of disclosure, and not by way of limitation, the detailed description of the invention is divided into the subsections which follow.

5.1. Misexpression of Ataxin-1

The current invention provides methods for generating SCA-1 phenotypes in Drosophila by ectopic expression of an ataxin-1 gene, and transgenic Drosophila which ectopically express an ataxin-1 gene. Ectopic expression, including misexpression or overexpression, of a normal or altered ataxin-1 gene in Drosophila is a method for the analysis of gene function (Brand et al., 1994, Methods in Cell Biology 44:635-654; Hay et al., 1997, Proc. Natl. Acad. Sci. U.S.A. 94(10):5195-200).

In one embodiment, the normal ataxin-1 protein comprises a polyglutamine repeat having 6-19 glutamine residues. In specific modes of the embodiment, the normal ataxin-1 protein comprises a polyglutamine repeat having 6-8 glutamine residues, 8-10 glutamine residues, 10-13 glutamine residues, 13-16 glutamine residues, or 16-19 glutamine residues. In other embodiments, the transgene encodes ataxin-1 comprising a polyglutamine repeat having 20-40 glutamine residues and 1-4 histidine residues. In specific modes of the embodiment, the normal ataxin-1 protein comprises a polyglutamine repeat having 20-25 glutamine residues, 25-30 glutamine residues, 30-35 glutamine residues, or 35-40 glutamine residues. In yet other specific modes of the embodiment, modes of the embodiment, the normal ataxin-1 protein comprises a polyglutamine repeat having 1 histidine residue, 2 histidine residues, 3 histidine residues, or 4 histidine residues.

In another embodiment, the mutant ataxin-1 protein comprises a polyglutamine repeat having 39-82 glutamine residues. In specific modes of the embodiment, the mutant ataxin-1 protein comprises a polyglutamine repeat having 39-45 glutamine residues, 45-55 glutamine residues, 55-65 glutamine residues, 65-75 glutamine residues, or 75-82 glutamine residues. In yet other embodiments, the mutant ataxin-1 protein has greater than 82 glutamine residues, for example 83-90, 91-105, 106-125, 126-150 or 151-200 glutamine residues.

Such transgenic Drosophila are created that contain gene fusions of the coding regions of ataxin-1 genes (from either genomic DNA or cDNA) operably joined to a specific promoter and transcriptional enhancer whose regulation has preferably been well characterized, preferably a heterologous promoter/enhancer that is spatially and/or temporally regulated. Examples of promoters/enhancers that can be used to drive such misexpression of ataxin-1 genes include, but are not limited to, the heat shock promoters/enhancers from the hsp70 and hsp83 genes, useful for temperature induced expression; tissue specific promoters/enhancers such as the sevenless promoter/enhancer (Bowtell et al., 1988, Genes Dev. 2(6):620-34), the eyeless promoter/enhancer (Bowtell et al., 1991, Proc. Natl. Acad. Sci. U.S.A. 88(15):6853-7), and glass-responsive promoters/enhancers (Quiring et al., 1994, Science 265:785-9) useful for expression in the eye; enhancers/promoters derived from the dpp or vestigal genes useful for expression in the wing (Stachling-Hampton et al., 1994, Cell Growth Differ. 5(6):585-93; Kim et al., 1996, Nature 382:133-8); promoters/enhancers derived from the elav (Yao and White, 1994, J. Neurochem 63(1):41-51), Appl (Martin-Morris and White, 1990, Development 110(1):185-95), and nirvana (Sun et al., 1999, Proc. Nat'l Acad. Sci. U.S.A. 96:10438-43) genes useful for expression in the central nervous system; and binary control systems employing exogenous DNA regulatory elements and exogenous transcriptional activator proteins, useful for testing the misexpression of genes in a wide variety of developmental stage-specific and tissue-specific patterns. Two examples of binary exogenous regulatory systems include the UAS/GAL4 system from yeast (Hay et al., 1997, Proc. Natl. Acad. Sci. U.S.A. 94(10):5195-200; Ellis et al., 1993, Development 119(3):855-65) and the “Tet system” derived from E. coli, both of which are described below. It is readily apparent to those skilled in the art that additional binary systems can be used which are based on other sets of exogenous transcriptional activators and cognate DNA regulatory elements in a manner similar to that for the UAS/GAL4 system and the Tet system.

In a specific embodiment, the UAS/GAL4 system is used. This system is a well-established and powerful method of misexpression in Drosophila which employs the UAS upstream regulatory sequence for control of promoters by the yeast GAL4 transcriptional activator protein (Brand and Perrimon, 1993, Development 118(2):401-15). In this approach, transgenic Drosophila, termed “target” lines, are generated where the gene of interest (e.g., an ataxin-1 gene) to be misexpressed is operably fused to an appropriate promoter controlled by UAS. Other transgenic Drosophila strains, termed “driver” lines, are generated where the GAL4 coding region is operably fused to promoters/enhancers that direct the expression of the GAL4 activator protein in specific tissues, such as the eye, antenna, wing, or nervous system. The gene of interest is not expressed in the so-called target lines for lack of a transcriptional activator to “drive” transcription from the promoter joined to the gene of interest. However, when the UAS-target line is crossed with a GAL4 driver line, misexpression of the gene of interest is induced in resulting progeny in a specific pattern that is characteristic for that GAL4 line. The technical simplicity of this approach makes it possible to sample the effects of directed misexpression of the gene of interest in a wide variety of tissues by generating one transgenic target line with the gene of interest, and crossing that target line with a panel of pre-existing driver lines. A very large number of specific GAL4 driver lines have been generated previously and are available for use with this system.

In specific embodiments of the foregoing methods, the expression of ataxin-1 gene can be controlled at the temporal and spatial level, by using a conditional GAL4 protein, such as RU486-dependent GAL4 protein(also known as GeneSwitch). As the UAS/GAL4 system, the GeneSwitch system is a binary expression system. In this approach, as in the UAS/GAL4 binary expression system, transgenic Drosophila, termed “target” lines, bear transgenes in which the gene to be misexpressed (e.g., ataxin-1) is operably fused to an appropriate promoter controlled by the Upstream Activating Sequence (UAS). The second class of transgenic Drosophila, termed “target” lines bear transgenes that express the RU486-dependent GAL4 under the control of a promoter of choice. The RU486-dependent GAL4 coding region can be operably fused to promoter/enhancer sequences that direct the expression of RU486-dependent GAL4 in specific tissues, including, but not limited to, the eye, antenna, wing, and nervous system. Alternatively, the RU486-dependent GAL4 coding sequence can be operably fused to a basic promoter, e.g., a heat shock promoter, within the ransgene, and subsequently the transgene is inserted into the Drosophila genome where the expression of RU486-dependent GAL4 is under the control of enhancer elements neighboring to the transgene. As RU486-dependent GAL4 is transcriptionally active only in the presence of its activator RU486 (mifepristone), the timing of RU486-dependent GAL4 activity can be determined by the administration of RU486. When a target line is crossed with a driver line, the progeny carries the transgene encoding the gene to be expressed and a transgene encoding the RU486-dependent GAL4. However, in the absence of RU486 (mifepristone) the gene of interest is not expressed. Only if RU486 (mifepristone) is administered, e.g., by feeding or “larval bathing”, expression of the gene of interest is induced. The combination of temporal and spatial control of expression, allows to obviate viability problems that may be associated with the global and/or continuous expression of the gene of interest. Furthermore, in certain embodiments of the invention, it may be of interest to start or to discontinue the expression of the gene of interest at a certain time of development in order to interfere only with a specific developmental process(es).

In a second embodiment, a related method of directed misexpression in Drosophila is used, that makes use of a tetracycline-regulated gene expression from E. coli, referred to as the “Tet system”. In this case, transgenic Drosophila driver lines are generated where the coding region for a tetracycline-controlled transcriptional activator (tTA) is operably fused to promoters/enhancers that direct the expression of tTA in a tissue-specific and/or developmental stage-specific manner. Also, transgenic Drosophila target lines are generated where the coding region for the gene of interest to be misexpressed (e.g., an ataxin-1 gene) is operably fused to a promoter that possesses a tTA-responsive regulatory element. Here again, misexpression of the gene of interest can be induced in progeny from a cross of the target line with any driver line of interest; moreover, the use of the Tet system as a binary control mechanism allows for an additional level of tight control in the resulting progeny of this cross. When Drosophila food is supplemented with a sufficient amount of tetracycline, it completely blocks expression of the gene of interest in the resulting progeny. Expression of the gene of interest can be induced at will simply by removal of tetracycline from the food. Also, the level of expression of the gene of interest can be adjusted by varying the level of tetracycline in the food. Thus, the use of the Tet system as a binary control mechanism for misexpression has the advantage of providing a means to control the amplitude and timing of misexpression of the gene of interest, in addition to spatial control. Consequently, if a gene of interest (e.g., an ataxin-1 gene) has lethal or deleterious effects when misexpressed at an early stage in development, such as the embryonic or larval stages, the function of the gene of interest in the adult can still be assessed using the Tet system, by adding tetracycline to the food during early stages of development and removing tetracycline later so as to induce misexpression only at the adult stage.

5.2. Generation of Drosophila that Misexpress Ataxin-1

Methods for creation and analysis of transgenic Drosophila strains having modified expression of genes are well known to those skilled in the art (Brand et al., 1994, Methods in Cell Biology 44:635-654; Hay et al., 1997, Proc. Natl. Acad. Sci. USA 94(10):5195-200). Open reading frame regions encoding normal (e.g., ataxin-1 30Q) or mutant (e.g., including but not limited to ataxin-1 82Q) ataxin genes can be operably fused to a desired promoter, as described above, and the promoter-ataxin-1 gene fusion inserted into any appropriate Drosophila transformation vector for the generation of transgenic flies. Typically, such transformation vectors are based on a well-characterized transposable elements, for example the P element (Rubin and Spradling, 1982, Science 218:348-53), the hobo element (Blackman et al., 1989, Embo J. 8(1):211-7), mariner element (Lidholm et al., 1993, Genetics 134(3):859-68), the hermes element (O'Brochta et al., 1996, Genetics 20 142(3):907-14), Minos (Loukeris et al., 1995, Proc. Natl. Acad. Sci. USA 92(21):9485-9), or the PiggyBac element (Handler et al., 1998, Proc. Natl. Acad. Sci. USA 95(13):7520-5), where the terminal repeat sequences of the transposon that are required for transposition are incorporated into the transformation vector and arranged such that the terminal repeat sequences flank the transgene of interest (in this case a promoter-ataxin-1 gene fusion) as well as a marker gene used to identify transgenic animals. Most often, marker genes are used that affect the eye color of Drosophila, such as derivatives of the Drosophila white or rosy genes; however, in principle, any gene can be used as a marker that causes a reliable and easily scored phenotypic change in transgenic animals, and examples of other marker genes used for transformation include the yellow gene used as a marker that affects bristle pigmentation, and the forked gene as a marker that affects bristle morphology; Adh⁺ gene used as a selectable marker for the transformation of Adh⁻ strains; Ddc+ gene used to transform Ddc^{ts2} mutant strains; the lacZ gene of E. coli; the neomycin^{R} gene from the E. coli transposon Tn5; and the green fluorescent protein (GFP; Handler and Harrell, 1999, Insect Molecular Biology 8:449-457), which can be under the control of different promoter/enhancer elements, e.g., eye-, antenna-, wing-, leg-specific, or the poly-ubiquitin promoter/enhancer elements. Plasmid constructs for introduction of the desired transgene are coinjected into Drosophila embryos having an appropriate genetic background, along with a helper plasmid that expresses the specific transposase need to mobilized the transgene into the genomic DNA. Animals arising from the injected embryos (G0 adults) are selected, or screened manually, for transgenic mosaic animals based on expression of the marker gene phenotype and are subsequently crossed to generate fully transgenic animals (G1 and subsequent generations) that will stably carry one or more copies of the transgene of interest (e.g., the ataxin-1 transgene).

5.3. Analysis of Misexpression Phenotypes

After isolation of fruit flies carrying normal (including but not limited to ataxin-1 Q30) or mutant (including but not limited to ataxin-1 Q82) ataxin-1 gene(s) and, if necessary, by induction of ataxin-1 overexpression (for example by subjecting the animals to heat shock if the ataxin-1 gene is under the control of a heat shock promoter), animals are inspected for misexpression phenotypes, such as abnormal development, morphology, viability, or behavior, in order to determine the functioning of the ataxin-1 gene in Drosophila. Tissue from these animals can be analyzed histologically to determine morphological aberrations at the cellular and tissue levels. In particular, neural degeneration can be determined by the detection of loss or abnormality of the Purkinje cell layer. Alternatively, the presence of nuclear inclusions can be determined, and if present, the nuclear inclusions can be analyzed for the accumulation of molecular chaperones, ubiquitin or proteasomes, as described in Section 6, infra.

To analyze the effect of expression levels on the misexpression phenotype, in one embodiment, fruit flies are generated that are homozygous and heterozygous for the same ataxin-1 transgene insertion. In another embodiment, different lines are assayed, as the expression levels from one ataxin-1 transgenic line to another will vary due to local chromatin effects at the site of transgene insertion. Alternatively, if the ataxin-1 gene is under the control of a UAS element, the animals harboring the UAS-ataxin-1 target and the Gal4 driver line are cultured at different temperatures, as expression in this system increases with temperature. For example, for low levels of expression in one line, the animals are cultured at 18° C.; for intermediate levels of expression in the same line, the animals are cultured at 21-22° C.; and for high levels of expression in the same line, the animals are cultured at 25-29° C. In other embodiments, the expression of the ataxin-1 gene is under control of the GeneSwitch system. Drosophila bearing the UAS-ataxin-1 target transgene and the RU486-GAL4 are reared either in the presence or in the absence of RU486 (mifepristone) in order to induce or supress the expression of ataxin-1 (see Section 5.1, supra). In another embodiment, the ataxin-1 gene is under the control of a heat shock inducible promoter such as hsp70. Overexpression of the ataxin-1 gene can be induced by incubating transgenic flies at 30° C. In yet another embodiment, when the ataxin-1 transgene is expressed under the control of the Tet system, varying amounts of tetracycline are added to the animal food.

Additionally, the ataxin-1 overexpression phenotype can be examined to determine if it is cell autonomous or cell non-autonomous. For example, clonal analysis can then be used to determine whether the phenotype produced by the misexpression of the ataxin-1 gene is restricted to cells expressing the gene or whether misexpression of the ataxin-1 gene exerts a non-autonomous effect on neighboring cells. Methods of mitotic recombination of chromosomes in heterozygous flies can be used to generate mitotic clones of genetically homozygous cells that are well known to those skilled in the art, which include the use of X-rays or preferably FLP/FRT mediated recombination (Xu and Harrison, 1994, Methods in Cell Biology 44:655-681; Greenspan, 1979, In Fly Pushing: The Theory and Practice of Drosophila Genetics. Plainview, N.Y., Cold Spring Harbor Laboratory Press: pp. 103-124). These mitotic recombination techniques result in patches of cells, mitotic clones, that contain two or no copies of the gene or transgene of interest. Production of the overexpression/misexpression phenotype within cells in a clone having no copies of the gene or transgene of interest indicates that the effect is not cell autonomous.

5.4. Identifying Ataxin-1 Pathways and Phenotypes

This invention provides animal models which may be used in the identification and characterization of Drosophila genes which interact with normal or mutant ataxin-1. Identification and characterization of Drosophila genes which interact with normal or mutant ataxin-1 can elucidate the underlying cellular mechanisms that result in SCA-1, and provide a basis for novel diagnostics and therapeutics for SCA-1 and other polyglutamine related diseases.

The procedures involved in typical genetic modifier screens to define components of a genetic/biochemical pathway are well known to those skilled in the art and have been described elsewhere (see, e.g., Wolfner and Goldberg, 1994, Methods in Cell Biology 44:33-80; Karim et al., 1996, Genetics 143:315-329).

Transgenic Drosophila are provided which carry an ataxin-1 transgene under the control of a spatially or temporally regulated or regulatable control element, as described in Sections 5.1 or 5.2, supra. In one embodiment, the ataxin-1 transgenic Drosophila are crossed to animals having mutations in gene(s) whose mammalian homologs are suspected to play a role in the pathogenesis of SCA-1. Some examples of suspected proteins involved in SCA-1 pathogenesis include hsp70 molecular chaperone, ubiquitin, and the proteasome, because molecular chaperones and proteasome components are suspected to play a role in SCA-1 pathogenesis. Crosses can be performed between animals with an ataxin-1 Q82 transgene and animals with mutations in gene(s) suspected to play a role in SCA-1 pathogenesis. If appropriate mutants are not available, loss of function phenotypes can be generated as described in Section 5.4.3, infra. Alternatively, crosses can be performed between animals that harbor an ataxin-1 transgene and a transgene(s) for the misexpression of the gene(s) suspected to play a role in SCA-1 pathogenesis. The offspring of such crosses can be analyzed to determine whether the SCA-1 pathogenesis has been enhanced or suppressed. As demonstrated in Section 6, crosses between ataxin-1 Q82 and DF (3R) karD1 (a small deletion removing a cluster of hsp70 genes), hsc70-4 (a point mutation in the ATP-binding domain of hsp70 cognate 4 protein) and pros26 (a point mutation in the gene encoding a multicatalytic endopeptidase which is a component of the 20S core proteasome) demonstrate that misexpression of ataxin-1 82Q in these mutant backgrounds results in a more severe phenotype than that produced by the misexpression of ataxin-1 82Q in an otherwise normal genetic background. Similar crosses can also be performed for other genes that are suspected to encode proteins that interact with ataxin-1.

5.4.1. Screening for Mutations that Modify SCA-1 Phenotypes

In certain embodiment of the invention, modifiers of the SCA-1 phenotype produced by misexpression of ataxin-1 in Drosophila are identified in a screen that combines the ataxin-1 transgene with various mutations.

In one embodiment, the ataxin-1 transgenic Drosophila are crossed to mutagenized animals (produced using chemical, radiation or transposon mutagenesis). Examples of effective chemical mutagens include EMS, MMS, ENU, triethylamine, diepoxyalkanes, ICR-170, and formaldehyde; effective radiation mutagens include X-rays, gamma rays, and ultraviolet radiation. In other embodiments, the ataxin-1 transgenic Drosoophila are crossed to animals with randomly inserted P or EP elements, as described in Section 6, infra. The progeny of the cross are analyzed to determine whether SCA-1 disease progression has been modified.

In a pilot-scale genetic modifier screen in which the ataxin-1 transgenic Drosophila are chemically mutagenized or crossed to mutagenized animals, 10,000 or fewer mutagenized progeny are inspected; in a moderate size screen, 10,000 to 50,000 mutagenized progeny are inspected; and in a large scale screen, over 50,000 mutagenized progeny are inspected. Progeny exhibiting either enhancement or suppression of the original phenotype are immediately crossed to adults containing balancer chromosomes and used as founders of a stable genetic line. In addition, progeny of the founder adult are retested under the original screening conditions to ensure stability and reproducibility of the phenotype. Additional secondary screens may be employed, as appropriate, to confirm the suitability of each new modifier mutant line for further analysis. For example, newly identified modifier mutations can be tested directly for interaction with other genes of interest known to be involved or implicated in SCA-1 pathogenesis (including those identified in the modifier screens described in Section 6, infra), using methods described above. Also, the new modifier mutations can be tested for interactions with ataxin-1 in tissues other than those utilized in the primary screening assay. For example, if the primary screening assay utilizes a rough eye phenotype, the phenotype can be confirmed by examining neural degeneration in the central nervous system. The modifier can be tested for its interactions with genes in other pathways thought to be unrelated or distantly related to SCA-1 pathology, such as genes in the sevenless signaling pathway in the eye. New modifier mutations that exhibit specific genetic interactions with ataxin-1, but not interactions with genes in unrelated pathways, are of particular interest. Additionally, strains can be generated that carry the new modifier mutations of interest in the absence of the original ataxin-1 transgene to determine whether the new modifier mutation exhibits an intrinsic phenotype, independent of the ataxin-1 misexpression, which would provide further clues as to the normal function of the newly-identified modifier gene.

Each newly-identified modifier mutation can be crossed to other modifier mutations identified in the same screen to place them into complementation groups, which typically correspond to individual genes (Greenspan, 1997, In Fly Pushing: The Theory and Practice of Drosophila Genetics, Plainview, N.Y., Cold Spring Harbor Laboratory Press: pp. 23-46). Two modifier mutations are said to fall within the same complementation group if animals carrying both mutations in trans exhibit essentially the same phenotype as animals that are homozygous for each mutation individually.

5.4.2. Screening for Genes Whose Misexpression Modifies SCA-1

In other embodiments of the invention, modifiers of the SCA-1 phenotype can be identified by creating Drosophila which misexpress ataxin-1 and another gene in the same cells. This is best achieved by using the modular misexpression system described above, for example by utilizing components of the GAL4/UAS system to perform the above mentioned screens. In this case a modified P element, termed an EP element, is genetically engineered to contain a GAL4-responsive UAS element and promoter, or an tTA-responsive tet element and promoter, and this engineered transposon is used to randomly tag genes by insertional mutagenesis (similar to the method of P mutagenesis described above). Thousands of transgenic Drosophila strains, termed EP lines, can thus be generated each containing a specific UAS- or tet-tagged gene. This approach takes advantage of a well-recognized insertional preference of P elements to insert at the 5′-ends of genes. Consequently, many of the genes that have been tagged by insertion of EP elements become operably fused to a GAL4- or tTA-regulated promoter, and increased expression or misexpression of the randomly tagged gene can be induced by crossing it to a GAL4 driver gene (as described in Section 5.1, supra).

Thus, systematic gain-of-function genetic screens for modifiers of the SCA-1 phenotypes induced by misexpression of ataxin-1 can be performed as follows. Drosophila having an ataxin-1 transgene under the control of a regulatory element that functions as a target for a driver in a binary expression system are crossed to animals having random genomic insertions of a regulatory element that functions as a target for the same or different binary expression system driver. Many feasible genetic permutations can produce progeny with all driver and target transgenes required for the screen.

In a specific embodiment, a large battery of thousands of Drosophila EP lines can be crossed into a genetic background containing a misexpressed ataxin-1 gene, and further containing an appropriate GAL4 driver transgene. The progeny of this cross can be inspected for enhancement or suppression of the SCA-1 phenotype induced by misexpression of the ataxin-1 transgene. If the gene in the EP line which the UAS element has randomly inserted is involved in SCA-1 pathogenesis, its misexpression under the control of the UAS element in the presence of Gal4 will result in an enhancement or suppression of the SCA-1 phenotype. The UAS transgene can be used as a basis for mapping and cloning the SCA-1 modifier gene into which it is inserted. Progeny that exhibit an enhanced or suppressed phenotype can be crossed further to verify the reproducibility and specificity of this genetic interaction with the ataxin-1 transgene. EP insertions that demonstrate a specific genetic interaction with ataxin-1 have therefore physically tagged a new gene that genetically interacts with ataxin-1. The new modifier gene can be identified and sequenced using PCR or hybridization screening methods that allow the isolation of the genomic DNA adjacent to the position of the EP element insertion.

In other embodiments, the ataxin-1 transgene is under the control of a UAS element and the EP lines inserted element comprises a tTA regulatory element. Animals that harbor the UAS-ataxin-1 transgene and an appropriate driver line (e.g., gmr-Gal4, in which Gal4 is expressed under the control of a regulatory element from the glass gene) are crossed to animals with the EP lines of the tTA regulatory element. The progeny are cultured on tetracycline containing media, allowing the simultaneous expression of ataxin-1 and the gene in which the tTA regulatory element has randomly inserted. If the gene in which the tTA regulatory element is inserted is involved in SCA-1 pathogenesis, its misexpression under the control in the presence of teteracycline will result in an enhancement or suppression of the SCA-1 phenotype. The tTA transgene can be used as a basis for mapping and cloning the SCA-1 modifier gene into which it is inserted. In the present embodiment, strains carrying the driver and target genes of interest, including the ataxin-1 transgene, which in this context is a target gene, can be generated by cross breeding animals carrying the genes, followed by selection of recombinant progeny that carry the desired transgenes based on the markers harbored by the individual constructs containing the trangenes. Progeny that exhibit an enhanced or suppressed phenotype can be crossed further to verify the reproducibility and specificity of this genetic interaction with the ataxin-1 transgene. EP/tTA insertions that demonstrate a specific genetic interaction with ataxin-1 have therefore physically tagged a new gene that genetically interacts with ataxin-1. The new modifier gene can be identified and sequenced using PCR or hybridization screening methods that allow the isolation of the genomic DNA adjacent to the position of the EP element insertion.

5.4.3. Generating Loss of Function Phenotypes of SCA-1 Modifier Genes

Once genes whose overexpression results in a modification of SCA-1 pathogenesis are identified, the interaction between loss of function phenotypes of these genes and the SCA-1 phenotype can be assessed. Loss of function genotypes may be available from Drosophila stock centers or created by traditional genetics methods (see Greenspan, 1979, In Fly Pushing: The Theory and Practice of Drosophila Genetics. Plainview, N.Y., Cold Spring Harbor Laboratory Press); alternatively, molecular disruption of gene expression can yield information on the existence of such genetic interactions while circumventing laborious mutagenesis screens. The molecular disruption methods described herein can also be used to test the interaction between ataxin-1 and a gene suspected to play a role in SCA-1 pathogenesis but for which a genetic mutation is not available. In such experiments, molecular disruption methods are conducted in parallel in normal animals and ataxin-1 misexpressing animals, to determine the extent to which a suppression or enhancement of the SCA-1 pathogenesis is a specific to the misexpression of ataxin-1.

In one embodiment, antisense RNA methods can be performed (Schubiger and Edgar, 1994, Methods in Cell Biology 44:697-713). One form of the antisense RNA method involves the injection of embryos with an antisense RNA that is partially homologous to the gene of interest (in this case a SCA-1 modifier gene or suspected SCA-1 modifier gene). Another form of the antisense RNA method involves expression of an antisense RNA partially homologous to the gene of interest by operably joining a portion of the gene of interest in the antisense orientation to a powerful promoter that can drive the expression of large quantities of antisense RNA, either generally throughout the animal or in specific tissues. Examples of powerful promoters that can be used in this strategy of antisense RNA include heat shock gene promoters or promoters controlled by potent exogenous transcription factors, such as GAL4 and tTA as described above. Antisense RNA-generated loss of function phenotypes have been reported previously for several Drosophila genes including cactus, pecanex, and Krupple (LaBonne et al., 1989, Dev. Biol. 136(1):1-16; Schuh and Jackle, 1989, Genome 31(1):422-5; Geisler et al., 1992, Cell 71(4):613-21).

In a second embodiment, loss of function phenotypes are generated by cosuppression methods (Bingham, 1997, Cell 90(3):385-7; Smyth, 1997, Curr. Biol. 7(12):793-5; Que and Jorgensen, 1998, Dev. Genet. 22(1):100-9). Cosuppression is a phenomenon of reduced gene expression produced by expression or injection of a sense strand RNA corresponding to a partial segment of the gene of interest. Cosuppression effects have been employed extensively in plants to generate loss of function phenotypes, and there is report of cosuppression in Drosophila where reduced expression of the Adh gene was induced from a white-Adh transgene (Pal-Bhadra et al., 1997, Cell 90(3):479-90).

In a third embodiment, loss of function phenotypes are generated by double-stranded RNA interference. This method is based on the interfering properties of double-stranded RNA derived from the coding regions of genes. Termed dsRNAi, this method has proven to be of great utility in genetic studies of the nematode C. elegans (see Fire et al., 1998, Nature 391:806-811) and, more recently, in genetic studies in Drosophila, both during embryogenesis (see, e.g., Kennerdell et al., 1998, Cell 95:1017-26) and during later development (Lam and Thummel, 2000, Curr Biol 10(16):957-63). In a preferred embodiment of this method, complementary sense and antisense RNAs derived from a substantial portion of a gene of interest are synthesized in vitro. Phagemid DNA templates containing cDNA clones of the gene of interest (i.e. a SCA-1 modifier gene) are inserted between opposing promoters for T3 and T7 phage RNA polymerases. Alternatively, one can use PCR products amplified from coding regions of SCA-1 modifier genes, where the primers used for the PCR reactions are modified by the addition of phage T3 and T7 promoters. The resulting sense and antisense RNAs are annealed in an injection buffer, and the double-stranded RNA injected or otherwise introduced into animals. Progeny of the injected animals are then inspected for phenotypes of interest.

5.5. Cloning Drosophila SCA-1 Modifier Genes

Once a modifier gene of SCA-1 is identified, it can be cloned for molecular analysis and for identification of vertebrate homologs.

For non-P element based mutations, the region containing the mutation is mapped to a precise genetic locus. The mutant is initially mapped to a specific chromosome using balancer strains (for example, wR13 or ywR13). Once the chromosome bearing the mutation is identified, the mutant strain is crossed to a deficiency collection of that chromosome to map the gene to a discrete genetic region within the chromosome. The strain is then crossed to P-element lines in that region to identify a P-element mutant that fails to complement the mutation in the SCA-1 modifier gene. Once such a P-element line is identified, the SCA-1 modifier gene can be cloned using polymerase chain reaction (PCR)-based methods.

In one embodiment, polymerase chain reaction (PCR) is used to amplify the desired sequence. Genomic DNA of a SCA-1 modifier Drosophila P element or, if the SCA-1 modifier is an EP strain, the EP element can be recovered by standard DNA extraction techniques. The regions flanking the P or EP elements can be recovered by digesting the genomic DNA with the appropriate restriction enzyme and then ligating to circularize the restriction fragments. A suitable cell line such as DH5α can be transformed by electroporation using standard procedures. The resulting colonies will have acquired the circularized restriction fragment containing the selectable marker, the bacterial origin of replication, one P element inverted repeat, and a variable amount of flanking genomic DNA. Plasmids can then be sequenced by standard protocols using a primer designed to the P element inverted repeat.

In another embodiment, the regions flanking the P or EP elements can be determined by the use of inverse PCR. Genomic DNA of an enhanced or suppressed SCA-1 fly can be recovered using standard DNA extraction techniques. The regions flanking the P or EP elements can be recovered by digesting the genomic DNA with the appropriate restriction enzyme and then ligated to circularize the restriction fragments. PCR can then be performed using standard methods by use of a Perkin-Elmer Cetus thermal cycler and Taq polymerase (e.g., Gene AMP™). The PCR product can then be sequenced using standard protocols.

If no non-complementing P-element or EP-element is found, several other methods known to the skilled artisan, such as, but not limited to, meiotic mapping, can be used to identify the position of the mutation. Once the mutation is mapped, DNA of the chromosomal region containing the mutation can be obtained, for example in the form of a bacterial artificial chromosome (see, e.g., Hoskins et al., 2000, Science 287:2271-74), P1 clones (see, e.g., Kimmerly et al., Genome Res. 6:414), or another recombinant form (see, e.g., Adams et al., 2000, Science 287:2185-95, describing the sequencing of the Drosophila genome inter alia from plasmids containing genomic DNA). Alternatively, the region of interest can be amplified by PCR. The DNA corresponding to the genomic region of interest can then be analyzed by heteroduplex analysis or single-strand conformational polymorphism (“SSCP”) to identify to exact nucleotide position of the mutation in the genome. A high throughput method of detecting mutations that can be used to accomplish this purpose is parallel capillary electrophoresis (Larsen et al., 2000, Comb. Chem. High Throughput Screen 3:393-409), which detects single base pair mismatches in heteroduplexes.

The above-described methods are not meant to limit the following general description of methods by which clones of Drosophila SCA-1 modifier genes may be obtained.

5.6. Cloning Non-Drosophila SCA-1 Modifier Genes

The present invention further provides homologs, preferably vertebrate homologs, more preferably mammalian homologs, most preferably human homologs, of SCA-1 modifier genes identified in Drosophila, for use as SCA-1 diagnostics and therapeutics, as well as for screening for compounds that inhibit SCA-1 and that are believed to be useful in treating of preventing neurodegenerative disorders.

Homologs of SCA-1 modifier genes include but are not limited to those molecules comprising regions that are substantially homologous to the SCA-1 modifier molecule or fragment thereof (e.g., in various embodiments, at least 60% or 70% or 80% or 90% or 95% identity over an amino acid sequence of identical size without any insertions or deletions or when compared to an aligned sequence in which the alignment is done by a computer homology program known in the art) or whose encoding nucleic acid is capable of hybridizing to a nucleic acid encoding a SCA-1 modifier protein, under high stringency, moderate stringency, or low stringency conditions.

With the availability of the human genome project and the identification of a large number of genes from non-human species, a search of available databases using computer algorithms for sequence comparison is believed to lead to the identification of a vertebrate homolog of a SCA-1 modifier gene.

To determine the percent identity of two amino acid sequences or of two nucleic acids, e.g. between the sequences of a Drosophila SCA-1 modifier gene and sequences from other organisms, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding a SCA-1 modifier protein. BLAST protein searches can be performed with the XBLAST program, score=50, wordlength=3 to obtain amino acid sequences homologous to a SCA-1 modifier protein. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci., 10:3-5; and FASTA described in Pearson and Lipman, 1988, Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al., 1996, Methods Enzymol. 266:383-402.

The percent identity between two sequences can be determined using techniques similar to those described above, with or without allowing gaps. In calculating percent identity, only exact matches are counted.

Once a homolog of a SCA-1 modifier gene is identified in a computer database, the homolog can be cloned by PCR amplification from a suitable source, for example a cDNA or genomic library.

In the other embodiments, a homolog of a SCA-1 modifier gene is cloned by expression cloning (a technique well known in the art). An expression library is constructed by any method known in the art. For example, mRNA is isolated, cDNA is made and ligated into an expression vector (e.g., a bacteriophage derivative) such that it is capable of being expressed by the host cell into which it is then introduced. Various screening assays can then be used to select for the expressed SCA-1 modifier product. In one embodiment, antibodies against the product of the SCA-1 modifier gene can be used for selection.

In another embodiment, PCR using degenerate oligonucleotides is used to amplify the desired sequence from a genomic or cDNA library of the species (and tissue) of interest. Oligonucleotide primers representing the Drosophila SCA-1 modifier sequences, or consensus sequences of the SCA-1 modifier homologs (derived from a comparison of the Drosophila modifier and homologs from other species), preferably based on amino acid sequences of minimal degeneracy, can be used as primers in PCR.

In other embodiments, a vertebrate homolog of a SCA-1 modifier can be identified by screening genomic or cDNA libraries of the desired vertebrate species with a Drosophila SCA-1 modifier. Homlogs of a SCA-1 modifier nucleic acid will hybridize under conditions of low, more preferably moderate, and most preferably high stringency hybridization, to a Drosophila SCA-1 modifier nucleic acid.

By way of example and not limitation, procedures using such conditions of low stringency for regions of hybridization of over 90 nucleotides are as follows (see also Shilo and Weinberg, 1981, Proc. Natl. Acad. Sci. U.S.A. 78,:6789-6792). Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art.

Also, by way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g., length of probe, GC content of probe, etc.), the relatedness of the species to Drosophila, the availability in the art of known homologs and the interrelatedness of their sequences, and can be determined by one of skill in the art.

The above-described methods are not meant to limit the following general description of methods by which clones of non-Drosophila SCA-1 modifier genes may be obtained.

Any vertebrate cell potentially can serve as the nucleic acid source for molecular cloning of a SCA-1 modifier gene. The nucleic acid sequences encoding SCA-1 modifier proteins may be isolated from vertebrate, including mammalian and avian sources. Preferred mammalian sources include but are not limited to human and additional primate sources, porcine, bovine, feline, equine, canine, etc. The DNA may be obtained by standard procedures known in the art from cloned DNA (e.g., a DNA “library”), by chemical synthesis, by cDNA cloning, or by the cloning of genomic DNA, or fragments thereof, purified from the desired cell (see e.g., Sambrook et al., 1989, Molecular Cloning, A Laboratory Manual, 2d Ed., Vol. I, II, Cold Spring Harbor Laboratory Press, Cold Spring Harbor, N.Y.; Glover, ed., 1985, DNA Cloning: A Practical Approach, MRL Press, Ltd., Oxford, U.K.).

5.7. Identification of Full Length SCA-1 Modifier Genes

Once a part of a Drosophila or non-Drosophila SCA-1 modifier is identified and propagated in a suitable cloning vector, the full gene can then be determined. The partial cloned sequence can be used to screen either a cDNA or genomic DNA library. Clones derived from the cDNA library will contain only exon sequences whereas clones derived from the genomic library will contain regulatory and intron DNA regions in addition to the coding regions. Only DNA fragment containing the identical sequence will hybridize if performed under high stringent conditions. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

Clones derived from genomic DNA may contain regulatory and intron DNA regions in addition to coding regions; clones derived from cDNA will contain only exon sequences. Whatever the source, the gene should be molecularly cloned into a suitable vector for propagation of the gene.

The cloning vector used for propagating the gene include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives or the Bluescript vector (Stratagene USA, La Jolla, Calif.). The insertion into a cloning vector can, for example, be accomplished by ligating the DNA fragment into a cloning vector which has complementary cohesive termin. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and a SCA-1 modifier gene may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shot gun” approach. Enrichment for the desired gene, for example, by size fractionization, can be done before insertion into the cloning vector.

In an additional embodiment, the desired gene may be identified and isolated after insertion into a suitable cloning vector using a strategy that combines a “shot gun” approach with a “directed sequencing” approach. Here, for example, the entire DNA sequence of a specific region of the genome, such as a sequence tagged site (STS), can be obtained using clones that molecularly map in and around the region of interest.

In specific embodiments, transformation of host cells with recombinant DNA molecules that incorporate a SCA-1 modifier gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

Nucleic acids encoding derivatives and analogs of SCA-1 modifier proteins, and SCA-1 modifier protein antisense nucleic acids are additionally provided. As is readily apparent, as used herein, a “nucleic acid encoding a fragment or portion of a SCA-1 modifier protein” shall be construed as referring to a nucleic acid encoding only the recited fragment or portion of the SCA-1 modifier protein and not the other contiguous portions of the SCA-1 modifier protein as a continuous sequence. The instant invention include those encoded amino acid sequences with functionally equivalent amino acids, as well as those encoding SCA-1 modifier derivatives or analogs.

5.8. Expression of Drosophila SCA1 Modifier Genes

The nucleotide sequence coding for a SCA-1 modifier protein or a functionally active analog or fragment or other derivative thereof (see Section 5.6), can be inserted into an appropriate expression vector, i.e., a vector which contains the necessary elements for the transcription and translation of the inserted protein-coding sequence. The necessary transcriptional and translational signals can also be supplied by the native SCA-1 modifier gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In yet another embodiment, a fragment of a SCA-1 modifier protein comprising one or more domains of the SCA-1 modifier protein is expressed.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of a nucleic acid sequence encoding a SCA-1 modifier protein or peptide fragment may be regulated by a second nucleic acid sequence so that the SCA-1 modifier protein or peptide is expressed in a host transformed with the recombinant DNA molecule. For example, expression of a SCA-1 modifier protein may be controlled by any promoter/enhancer element known in the art. A promoter/enhancer may be homologous (i.e. native) or heterologous (i.e. not native). Promoters which may be used to control SCA-1 modifier gene expression include, but are not limited to, the SV40 early promoter region (Benoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), prokaryotic expression vectors such as the β-lactamase promoter (Villa-Kamaroffet al., 1978, Proc. Natl. Acad. Sci. U.S.A. 75:3727-3731), or the lac promoter (DeBoer et al., 1983, Proc. Natl. Acad. Sci. U.S.A. 80:21-25; Scientific American, 1980, 242:74-94), plant expression vectors comprising the nopaline synthetase promoter region (Herrera-Estrella et al., Nature 303:209-213), the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120), promoter elements from yeast or other fungi such as the Gal4-responsive promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); a gene control region which is active in pancreatic beta cells (Hanahan, 1985, Nature 315:115-122), an immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mamrary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

In a specific embodiment, a vector is used that comprises a promoter operably linked to a SCA-1 modifier gene nucleic acid, one or more origins of replication, and, optionally, one or more selectable markers (e.g., an antibiotic resistance gene).

In a specific embodiment, an expression construct is made by subcloning a SCA-1 modifier coding sequence into the EcoRI restriction site of each of the three pGEX vectors (Glutathione S-Transferase expression vectors; Smith and Johnson, 1988, Gene 7:31-40). This allows for the expression of the SCA-1 modifier protein product from the subclone in the correct reading frame.

Expression vectors containing SCA-1 modifier gene inserts can be identified by three general approaches: (a) nucleic acid hybridization; (b) presence or absence of “marker” gene functions; and (c) expression of inserted sequences. In the first approach, the presence of a SCA-1 modifier gene inserted in an expression vector can be detected by nucleic acid hybridization using probes comprising sequences that are homologous to an inserted SCA-1 modifier gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a SCA-1 modifier gene in the vector. For example, if the SCA-1 modifier gene is inserted within the marker gene sequence of the vector, recombinants containing the SCA-1 modifier insert can be identified by the absence of the marker gene function. In the third approach, recombinant expression vectors can be identified by assaying the SCA-1 modifier product expressed by the recombinant. Such assays can be based, for example, on the physical or functional properties of the SCA-1 modifier protein in in vitro assay systems, e.g., binding with ataxin-1.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda phage), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered SCA-1 modifier protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce a non-glycosylated core protein product. Expression in yeast will produce a glycosylated product. Expression in animal cells can be used to ensure “native” glycosylation of a heterologous protein. Furthermore, different vector/host expression systems may effect processing reactions to different extents.

In other specific embodiments, the SCA-1 modifier protein, fragment, analog, or derivative may be expressed as a fusion, or chimeric protein product (comprising the protein, fragment, analog, or derivative joined via a peptide bond to a heterologous protein sequence (of a different protein)). A chimeric protein may include fusion of the SCA-1 modifier protein, fragment, analog, or derivative to a second protein or at least a portion thereof, wherein a portion is one (preferably 10, 15, or 20). or more amino acids of said second protein. The second protein, or one or more amino acid portion thereof, may be from a different Drosophila SCA-1 modifier protein or may be from a protein that is not a Drosophila SCA-1 modifier protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

5.9. Identification and Purification of SCA-1 Modifier Gene Products

In particular aspects, the invention provides amino acid sequences of SCA-1 modifier proteins and fragments and derivatives thereof which comprise an antigenic determinant of the SCA-1 modifier protein (i.e., can be recognized by an antibody) or which are otherwise functionally active, as well as nucleic acid sequences encoding the foregoing.

In specific embodiments, the invention provides fragments of a SCA-1 modifier protein consisting of at least 10 amino acids, 20 amino acids, 50 amino acids, or of at least 75 amino acids. Fragments, or proteins comprising fragments, lacking some or all of the foregoing regions of a SCA-1 modifier protein are also provided. Nucleic acids encoding the foregoing are provided. In specific embodiments, the foregoing proteins or fragments are not more than 25, 50, or 100 contiguous amino acids.

Once a recombinant which expresses the SCA-1 modifier gene sequence is identified, the gene product can be analyzed. This is achieved by assays based on the physical or functional properties of the product, including radioactive labeling of the product followed by analysis by gel electrophoresis, immunoassay, etc.

Once the SCA-1 modifier protein is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (see Section 5.7).

In another alternate embodiment, native SCA-1 modifier proteins can be purified from natural sources, by standard methods such as those described above (e.g., immunoaffinity purification).

In a specific embodiment of the present invention, such SCA-1 modifier proteins, whether produced by recombinant DNA techniques or by chemical synthetic methods or by purification of native proteins, as well as fragments and other derivatives, and analogs thereof, including proteins homologous thereto.

5.10. Structure of SCA-1 Modifier Genes and Proteins

The structure of SCA-1 modifier genes and proteins can be analyzed by various methods known in the art. Some examples of such methods are described below.

5.10.1. Nucleic Acid Analysis

The cloned DNA or cDNA corresponding to a SCA-1 modifier gene can be analyzed by methods including but not limited to Southern hybridization (Southern, 1975, J. Mol. Biol. 98:503-517), Northern hybridization (see e.g., Freeman et al., 1983, Proc. Natl. Acad. Sci. U.S.A. 80:4094-4098), restriction endonuclease mapping (Maniatis, 1982, Molecular Cloning, A Laboratory Manual, Cold Spring Harbor Laboratory Press, Cold Spring Harbor, N.Y.), and DNA sequence analysis. Accordingly, this invention provides nucleic acid probes recognizing a SCA-1 modifier gene. For example, polymerase chain reaction (PCR; U.S. Pat. Nos. 4,683,202, 4,683,195 and 4,889,818; Gyllenstein et al., 1988, Proc. Natl. Acad. Sci. U.S.A. 85:7652-7656; Ochman et al., 1988, Genetics 120:621-623; Loh et al., 1989, Science 243:217-220) followed by Southern hybridization with a SCA-1 modifier gene-specific probe can allow the detection of a SCA-1 modifier gene in DNA from various cell types. Methods of amplification other than PCR are commonly known and can also be employed. In one embodiment, Southern hybridization can be used to determine the genetic linkage of a SCA-1 modifier gene. Northern hybridization analysis can be used to determine the expression of a SCA-1 modifier gene. Various cell types, and in particular cells of the central nervous system, at various states of development or activity can be tested for SCA-1 modifier gene expression. The stringency of the hybridization conditions for both Southern and Northern hybridization can be manipulated to ensure detection of nucleic acids with the desired degree of relatedness to the specific SCA-1 modifier gene probe used. Modifications of these methods and other methods commonly known in the art can be used.

Restriction endonuclease mapping can be used to roughly determine the genetic structure of a SCA-1 modifier gene. Restriction maps derived by restriction endonuclease cleavage can be confirmed by DNA sequence analysis.

DNA sequence analysis can be performed by any techniques known in the art, including but not limited to the method of Maxam and Gilbert (1980, Meth. Enzymol. 65:499-560), the Sanger dideoxy method (Sanger et al., 1977, Proc. Natl. Acad. Sci. U.S.A. 74:5463), the use of T7 DNA polymerase (Tabor and Richardson, U.S. Pat. No. 4,795,699), or use of an automated DNA sequenator (e.g., Applied Biosystems, Foster City, Calif.).

5.10.2. analysis of SCA-1 Modifier Proteins

The amino acid sequence of a SCA-1 modifier protein can be derived by deduction from the DNA sequence, or alternatively, by direct sequencing of the protein, e.g., with an automated amino acid sequencer.

A SCA-1 modifier protein sequence can be further characterized by a hydrophilicity analysis (Hopp and Woods, 1981, Proc. Natl. Acad. Sci. U.S.A. 78:3824). A hydrophilicity profile can be used to identify the hydrophobic and hydrophilic regions of the SCA-1 modifier protein and the corresponding regions of the gene sequence which encode such regions.

Structural prediction analysis (Chou and Fasman, 1974, Biochemistry 13:222) can also be done, to identify regions of a SCA-1 modifier protein that assume specific secondary structures.

Manipulation, translation, and secondary structure prediction, open reading frame prediction and plotting, as well as determination of sequence homologies, can also be accomplished using computer software programs available in the art.

Other methods of structural analysis can also be employed. These include but are not limited to X-ray crystallography (Engstom, 1974, Biochem. Exp. Biol. 11:7-13), nuclear magnetic resonance spectroscopy (Clore and Gonenbom, 1989, CRC Crit. Rev. Biochem. 24:479-564) and computer modeling (Fletterick and Zoller, 1986, Computer Graphics and Molecular Modeling, in Current Communications in Molecular Biology, Cold Spring Harbor Laboratory Press, Cold Spring Harbor, N.Y.).

5.10.3. Antibodies

According to the invention, SCA-1 modifier protein, its fragments or other derivatives, or analogs thereof, may be used as an immunogen to generate antibodies which immunospecifically bind such an immunogen. Such antibodies include but are not limited to polyclonal, monoclonal, chimeric, single chain, Fab fragments, and an Fab expression library. In another embodiment, antibodies to a domain of a SCA-1 modifier protein are produced. In a specific embodiment, fragments of a SCA-1 modifier protein identified as hydrophilic are used as immunogens for antibody production.

Various procedures known in the art may be used for the production of polyclonal antibodies to a SCA-1 modifier protein or derivative or analog. For the production of antibody, various host animals can be immunized by injection with the native SCA-1 modifier protein, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed to a SCA-1 modifier protein sequence or analog thereof, any technique which provides for the production of antibody molecules by continuous cell lines in culture may be used. For example, the hybridoma technique originally developed by Kohler and Milstein, (Kohler and Milstein 1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (see e.g., PCT/US90/02545). According to the invention, human antibodies may be used and can be obtained by using human hybridomas (Cole et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for a SCA-1 modifier protein together with genes from a human antibody molecule of appropriate biological activity can be used; such antibodies are within the scope of this invention.

According to the invention, techniques described for the production of single chain antibodies (U.S. Pat. No. 4,946,778) can be adapted to produce SCA-1 modifier-specific single chain antibodies. An additional embodiment of the invention utilizes the techniques described for the construction of Fab′ expression libraries (Huse et al., 1989, Science 246:1275-1281) to allow rapid and easy identification of monoclonal Fab fragments with the desired specificity for SCA-1 modifier proteins, derivatives, or analogs.

Antibody fragments which contain the idiotype of the molecule can be generated by known techniques. For example, such fragments include but are not limited to, the F(ab′)₂ fragment which can be produced by pepsin digestion of the antibody molecule, the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art (e.g., enzyme-linked immunosorbent assay or ELISA). For example, to select antibodies which recognize a specific domain of a SCA-1 modifier protein, one may assay generated hybridomas for a product which binds to a SCA-1 modifier fragment containing such domain. For selection of an antibody that specifically binds a first SCA-1 modifier homolog but which does not specifically bind a different SCA-1 modifier homolog, one can select on the basis of positive binding to the first SCA-1 modifier homolog and a lack of binding to the second SCA-1 modifier homolog.

Antibodies specific to a domain of a SCA-1 modifier protein are also provided. Antibodies specific to an epitope of a SCA-1 modifier protein are also provided.

The foregoing antibodies can be used in methods known in the art relating to the localization and activity of the SCA-1 modifier protein sequences, e.g., for imaging these proteins, measuring levels thereof in appropriate physiological samples, in diagnostic methods, etc.

5.10.4. Derivatives and Analogs of SCA-1 Modifier Proteins

The invention further provides to SCA-1 modifier proteins, derivatives (including but not limited to fragments), analogs, and molecules of SCA-1 modifier proteins. Nucleic acids encoding SCA-1 modifier protein derivatives and protein analogs are also provided. In one embodiment, the SCA-1 modifier proteins are encoded by the SCA-1 modifier nucleic acids described in Section 5.1 above.

The production and use of derivatives and analogs related to a SCA-1 modifier protein are within the scope of the present invention. In a specific embodiment, the derivative or analog is functionally active, i.e., capable of exhibiting one or more functional activities associated with a full-length, wild-type SCA-1 modifier protein. As one example, such derivatives or analogs which have the desired immunogenicity or antigenicity can be used in immunoassays, for immunization, for inhibition of SCA-1 modifier activity, etc. Derivatives or analogs that retain, or alternatively lack or inhibit, a desired SCA-1 modifier protein property of interest can be used as inducers, or inhibitors, respectively, of such property and its physiological correlates. A specific embodiment relates to a SCA-1 modifier protein fragment that can be bound by an antibody against a SCA-1 modifier protein. Derivatives or analogs of a SCA-1 modifier protein can be tested for the desired activity.

In particular, SCA-1 modifier derivatives can be made by altering SCA-1 modifier sequences by substitutions, additions (e.g., insertions) or deletions that provide for functionally equivalent molecules. Due to the degeneracy of nucleotide coding sequences, other DNA sequences which encode substantially the same amino acid sequence as a SCA-1 modifier gene may be used in the practice of the present invention. These include but are not limited to nucleotide sequences comprising all or portions of a SCA-1 modifier gene which is altered by the substitution of different codons that encode a functionally equivalent amino acid residue within the sequence, thus producing a silent change. Likewise, the SCA-1 modifier derivatives include, but are not limited to, those containing, as a primary amino acid sequence, all or part of the amino acid sequence of a SCA-1 modifier protein including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are generally understood to be conservative substitutions.

In a specific embodiment of the invention, proteins consisting of or comprising a fragment of a SCA-1 modifier protein consisting of at least 10 (continuous) amino acids of the SCA-1 modifier protein is provided. In other embodiments, the fragment consists of at least 20 or at least 50 amino acids of the SCA-1 modifier protein. In specific embodiments, such fragments are not larger than 35, 100 or 200 amino acids. Derivatives or analogs of SCA-1 modifier proteins include but are not limited to those molecules comprising regions that are substantially homologous to a SCA-1 modifier protein or fragment thereof (e.g., in various embodiments, at least 60% or 70% or 80% or 90% or 95% identity over an amino acid sequence of identical size or when compared to an aligned sequence in which the aligmnent is done by a computer homology program known in the art) or whose encoding nucleic acid is capable of hybridizing to a coding SCA-1 modifier gene sequence, under high stringency, moderate stringency, or low stringency conditions.

The SCA-1 modifier derivatives and analogs can be produced by various methods known in the art. The manipulations which result in their production can occur at the gene or protein level. For example, a cloned SCA-1 modifier gene sequence can be modified by any of numerous strategies known in the art (Sambrook et al., 1989, Molecular Cloning, A Laboratory Manual, 2d ed., Cold Spring Harbor Laboratory Press, Cold Spring Harbor, N.Y.). The sequence can be cleaved at appropriate sites with restriction endonuclease(s), followed by further enzymatic modification if desired, isolated, and ligated in vitro. In the production of a modified gene encoding a derivative or analog of a SCA-1 modifier protein, care should be taken to ensure that the modified gene remains within the same translational reading frame as the native protein, uninterrupted by translational stop signals, in the gene region where the desired SCA-1 modifier protein activity is encoded.

Additionally, a SCA-1 modifier nucleic acid sequence can be mutated in vitro or in vivo, to create and/or destroy translation, initiation, and/or termination sequences, or to create variations in coding regions and/or to form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson et al., 1978, J. Biol. Chem. 253:6551), use of TAB® linkers (Pharmacia), PCR with primers containing a mutation, etc.

Manipulations of a SCA-1 modifier protein sequence may also be made at the protein level. Included within the scope of the invention are SCA-1 modifier protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In addition, analogs and derivatives of a SCA-1 modifier protein can be chemically synthesized. For example, a peptide corresponding to a portion of a SCA-1 modifier protein which comprises the desired domain, or which mediates the desired activity in vitro, can be synthesized by use of a peptide synthesizer. Furthermore, if desired, nonclassical amino acids or chemical amino acid analogs can be introduced as a substitution or addition into the SCA-1 modifier sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In a specific embodiment, a SCA-1 modifier protein derivative is a chimeric or fusion protein comprising a SCA-1 modifier protein or fragment thereof (preferably consisting of at least a domain or motif of the SCA-1 modifier protein, or at least 10 amino acids of the SCA-1 modifier protein) joined at its amino- or carboxy-terminus via a peptide bond to an amino acid sequence of a different protein. In specific embodiments, the amino acid sequence of the different protein is at least 6, 10, 20 or 30 continuous amino acids of the different proteins or a portion of the different protein that is functionally active. In one embodiment, such a chimeric protein is produced by recombinant expression of a nucleic acid encoding the protein (comprising a SCA-1 modifier-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of a SCA-1 modifier gene fused to any heterologous protein-encoding sequences may be constructed. A specific embodiment relates to a chimeric protein comprising a fragment of a SCA-1 modifier protein of at least six amino acids, or a fragment that displays one or more functional activities of the SCA-1 modifier protein.

5.11. Deagnostic Uses of Ataxin-1

Diagnostic uses of normal ataxin-1 are possible due to the discovery by the present inventors that overexpression of wild-type ataxin-1 30Q can induce SCA-1 pathogenesis (Section 6.5, infra). It was originally believed that the SCA-1 phenotype could only be caused by a mutant ataxin-1 protein with expanded polyglutamine repeats ataxin-1 with polyglutamine repeats of 39-82 glutamine residues (Zoghbi and Orr, 2000, Ann. Rev. Neurosci. 23:217-247). However, with the discovery that overexpression of wild-type ataxin-1 can lead to SCA-1 disease progression, diagnostic procedures can be developed to determine whether a patient is susceptible to SCA-1 by assaying the level of ataxin-1 in the central nervous system, for example in a patient or subject's tissue biopsy or cerebrospinal fluid.

Ataxin-1 nucleic acids and antibodies may be used to measure expression of normal ataxin-1. Overexpression of normal ataxin-1 can be indicative of a predisposition to SCA-1 or SCA-1 disease. In one embodiment of the invention, an immunoassay is carried out by contacting a sample derived from a patient with an anti-ataxin-1 antibody under conditions such that immunospecific binding can occur, and detecting or measuring the amount of any immunospecific binding by the antibody. In one embodiment, the antibody is a monoclonal antibody specific for a normal (non-expanded) ataxin-1 gene (encoding an ataxin-1 protein with 6-44 glutamine residues in the polyglutamine repeats, with those alleles with 20 or more glutamine residues in the polyglutamine tracts, the glutamine repeats are interrupted by one to four histidine residues (Zoghbi and Orr, 2000, Ann. Rev. Neurosci. 23:217-247)), i.e., the antibody shows preferential, or more preferably specific, binding to normal ataxin-1 relative to ataxin-1 with expanded polyglutamine repeats.

The immunoassays which can be used include but are not limited to competitive and non-competitive assay systems using techniques such as western blots, immunohisto-chemistry radioimmunoassays, ELISA, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

Ataxin-1 genes and related nucleic acid sequences and subsequences, including complementary sequences, can also be used in hybridization assays. Ataxin-1 nucleic acid sequences, or subsequences thereof, comprising about at least 8 nucleotides, more preferably at least 12 nucleotides, and most preferably at least 15 nucleotides, can be used as hybridization probes. Hybridization assays can be used to detect, prognose, diagnose, or monitor conditions, disorders, or disease states associated with aberrant changes in ataxin-1 expression. In particular, such a hybridization assay is carried out by a method comprising contacting a sample containing nucleic acid with a nucleic acid probe capable of hybridizing to ataxin-1 RNA, for example in a northern blot of RNA prepared from a tissue biopsy from a subject or patient, under conditions such that hybridization can occur, and detecting or measuring any resulting hybridization.

In other embodiments, PCR using primers, preferably primers that span the polyglutamine repeat of the ataxin-1 gene, are used in quantitative RT-PCR assays (see, e.g., Riedy et al., 1995, Biotechniques18(1):70-4, 76) for determining the expression levels of ataxin-1, or for simultaneously detecting the expression levels and, based on the size of the resulting PCR product, the expression levels of ataxin-1 and the presence of expanded polyglutamine repeats in a sample from a subject or patient.

In a preferred embodiment, levels of ataxin-1 mRNA or protein in a patient sample are detected or measured relative to the levels present in an analogous sample from a subject not having SCA-1. Increased levels indicate that the subject may develop, or have a predisposition to developing SCA-1.

Kits for diagnostic use are also provided, that comprise in one or more containers an anti-ataxin-1 antibody, and, optionally, a labeled binding partner to the antibody. Alternatively, the anti-ataxin-1 antibody can be labeled (with a detectable marker, e.g., a chemiluminescent, enzymatic, fluorescent, or radioactive moiety). A kit is also provided that comprises in one or more containers a nucleic acid probe capable of hybridizing to ataxin-1 RNA. In a specific embodiment, a kit can comprise in one or more containers a pair of primers, preferably each in the size range of 8-30 nucleotides, that are capable of priming amplification, e.g., by PCR (see e.g., Innis et al., 1990, PCR Protocols, Academic Press, Inc., San Diego, Calif.), ligase chain reaction (see EP 320,308) use of Qβ replicase, cyclic probe reaction, or other methods known in the art] under appropriate reaction conditions of at least a portion of an ataxin-1 nucleic acid. A kit for amplification of ataxin-1 RNA can optionally further comprise nucleotides and/or buffer(s) for the amplification procedure. A kit for amplification of ataxin-1 RNA can optionally further comprise reverse transcriptase enzyme for reverse transcribing ataxin-1 mRNA into cDNA for use as a template in the amplification procedure. A kit can optionally further comprise in a container a predetermined amount of a purified ataxin-1 protein or nucleic acid, e.g., for use as a quantitative standard or control.

5.12. Diagnostic Uses of SCA-1 Modifier Genes

The identification of SCA-1 modifiers as described herein will lead to the discovery of genes that contribute to the pathogenesis of SCA-1. As noted above, a SCA-1 enhancer gene is a gene whose loss of function results in more severe SCA-1 pathogenesis, or a gene whose misexpression or gain of function results in less severe SCA-1 pathogenesis. Once a SCA-1 enhancer gene is identified, the expression pattern of the SCA-1 gene is analyzed. SCA-1 enhancer genes that are normally expressed in central nervous system, and in particular, the cerebellar areas including Purkinje cells and dentate nucleus cells, are candidates for genes whose loss of function mutations contribute to SCA-1 and can be used in diagnostics. Specifically, analysis of the reduction of expression levels or activity of SCA-1 enhancer genes that are normally expressed in the nervous system can be used to diagnose a predisposition to SCA-1 or the actual disease. All SCA-1 enhancer genes, regardless of whether normally expressed in the central nervous system, are candidates for SCA-1 therapeutics. Specifically, the invention encompasses the use of SCA-1 therapeutics that are agonists of SCA-1 enhancer genes.

Conversely, a SCA-1 suppressor gene is a gene whose loss of function results in less severe SCA-1 pathogenesis, or a gene whose misexpression or gain of function results in more severe SCA-1 pathogenesis. Once a SCA-1 suppressor gene is identified, the expression pattern of the SCA-1 gene is analyzed. SCA-1 suppressor genes that are normally expressed in central nervous system, and in particular, the cerebellar areas including Purkinje cells and dentate nucleus cells, are candidates for genes whose loss of function mutations contribute to SCA-1 and can be used in diagnostics and therapeutics. Specifically, analysis of increased expression levels or activity of SCA-1 suppressor genes that are normally expressed in the nervous system can be used to diagnose a predisposition to SCA-1 or the actual disease. SCA-1 suppressor genes that are normally expressed in the nervous system, or that are misexpressed in the nervous system during the course of SCA-1, are candidates for SCA-1 therapeutics. Specifically, the invention encompasses the use of SCA-1 therapeutics that are antagonists of SCA-1 suppressor genes.

SCA-1 modifier proteins, SCA-1 modifier nucleic acids, and SCA-1 modifier antibodies may be used to detect, prognose, diagnose, or monitor SCA-1 disease or monitor the treatment thereof. In one embodiment of the invention, an immunoassay is carried out by a method comprising contacting a sample derived from a patient with an antibody specific for SCA-1 modifier under conditions such that immunospecific binding can occur, and detecting or measuring the amount of any immunospecific binding by the antibody. In a specific aspect, such binding of antibody, in tissue biopsies or cerebrospinal fluid extracts, can be used to detect aberrant expression of a SCA-1 modifier gene, where an aberrant level of a SCA-1 modifier gene is an indication of a diseased condition. As used herein, “aberrant levels” means increased levels of a SCA-1 suppressor gene or decreased levels of a SCA-1 enhancer gene relative to normal levels of gene expression.

The immunoassays which can be used include but are not limited to competitive and non-competitive assay systems using techniques such as western blots, immunohisto-chemistry radioimmunoassays, ELISA, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

SCA-1 modifier genes and related nucleic acid sequences and subsequences, including complementary sequences, can also be used in hybridization assays. SCA-1 modifier genes, or subsequences thereof, comprising about at least 8 nucleotides, can be used as hybridization probes. Hybridization assays can be used to detect, prognose, diagnose, or monitor SCA-1. In particular, such a hybridization assay is carried out by a method comprising contacting a sample containing nucleic acids prepared from a tissue biopsy or cerebrospinal fluid with a nucleic acid probe capable of hybridizing to a SCA-1 modifier nucleic acid, under conditions such that hybridization can occur, and detecting or measuring any resulting hybridization.

In specific embodiments, SCA-1 can be diagnosed, or its suspected presence can be screened for, or a predisposition to develop such disorder can be detected, by detecting increased levels of SCA-1 modifier protein, SCA-1 modifier RNA, or by detecting mutations in SCA-1 modifier RNA, DNA or SCA-1 modifier protein (e.g., translocations in SCA-1 modifier genes, truncations in SCA-1 modifier genes or proteins, changes in nucleotide or amino acid sequence relative to wild-type SCA-1 modifier genes or proteins, respectively) that cause altered expression or activity of a SCA-1 modifier gene or its product. By way of example, levels of SCA-1 modifier proteins can be detected by immunoassay, levels of SCA-1 modifier RNA can be detected by hybridization assays (e.g., Northern blots, in situ hybridization), SCA-1 modifier protein activity can be assayed by measuring binding activities in vivo or in vitro. Translocations, deletions, and point mutations in SCA-1 modifier genes can be detected by Southern blotting, FISH, RFLP analysis, SSCP, PCR using primers, sequencing of SCA-1 modifier genomic DNA or cDNA obtained from the patient, etc. In a preferred embodiment, PCR using primers specific to a SCA-1 modifier gene, are used in quantitative RT-PCR assays (see, e.g., Riedy et al., 1995, Biotechniques 18(1):70-4, 76) for determining the expression levels of the SCA-1 modifier gene.

Kits for diagnostic use are also provided, that comprise in one or more containers an anti-SCA-1 modifier protein antibody, and, optionally, a labeled binding partner to the antibody, such as a labeled secondary antibody. Alternatively, the anti-SCA-1 modifier protein antibody itself can be labeled (with a detectable marker, e.g., a chemiluminescent, enzymatic, fluorescent, or radioactive moiety). A kit is also provided that comprises in one or more containers a nucleic acid probe capable of hybridizing to a SCA-1 modifier RNA. In a specific embodiment, a kit can comprise in one or more containers a pair of primers, preferably each in the size range of 8-30 nucleotides, that are capable of priming amplification, e.g., by PCR (see e.g., Innis et al., 1990, PCR Protocols, Academic Press, Inc., San Diego, Calif.), ligase chain reaction (see EP 320,308) use of Qβreplicase, cyclic probe reaction, or other methods known in the art] under appropriate reaction conditions of at least a portion of a SCA-1 modifier nucleic acid. A kit for amplification of a SCA-1 modifier RNA can optionally further comprise nucleotides and/or buffer(s) for the amplification procedure. A kit for amplification of a SCA-1 modifier RNA can optionally further comprise reverse transcriptase enzyme for reverse transcribing a SCA-1 modifier mRNA into cDNA for use as a template in the amplification procedure. A kit can optionally further comprise in a container a predetermined amount of a purified a SCA-1 modifier protein or nucleic acid, e.g., for use as a quantitative standard or control.

5.13. Therapeutic Uses of SCA-1 Modifier Genes

The identification of SCA-1 modifiers as described herein will lead to the discovery of genes that can be used as SCA-1 therapeutics. Because of the common mechanisms and elements between SCA-1 and other neurodegenerative disrorders, the SCA-1 therapeutics identified by the methods disclosed herein are expected to be beneficial for the prevention or treatment of other polyglutamine diseases as well as non-polyglutamine diseases such as Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, prion diseases, a taupathies, spongiform encephalopathies, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorders, hypertension, neuropsychiatric disorder, schizophrenia, or schizoaffective disorders.

As noted above, a SCA-1 enhancer gene is a gene whose loss of function results in more severe SCA-1 pathogenesis, or a gene whose misexpression or gain of function results in less severe SCA-1 pathogenesis. All SCA-1 enhancer genes, regardless of whether normally expressed in the central nervous system, are candidates for SCA-1 therapeutics. Specifically, the invention encompasses the use of neurodegenerative therapeutics, including but not limited to SCA-1 therapeutics, that are agonists of SCA-1 enhancer genes.

A SCA-1 suppressor gene is a gene whose loss of function results in less severe SCA-1 pathogenesis, or a gene whose misexpression or gain of function results in more severe SCA-1 pathogenesis. Once a SCA-1 suppressor gene is identified, the expression pattern of the SCA-1 gene is analyzed. SCA-1 suppressor genes that are normally expressed in the nervous system, or that are misexpressed in the nervous system during the course of SCA-1, are candidates for SCA-1 therapeutics. Specifically, the invention encompasses the use of neurodegenerative therapeutics, including but not limited to SCA-1 therapeutics, that are antagonists of SCA-1 suppressor genes.

In accordance with the invention, the SCA-1 therapeutics, i.e., agonists of SCA-1 enhancers and antagonists of SCA-1 suppressors, are administered to human patients with SCA-1. In another embodiment, the compositions and formulations are administered to human subjects that do not have a SCA-1 as a preventative measure from developing the disease. It is appreciated, however, that the therapeutics developed using the principles described herein will be useful in treating diseases of other mammals, for example, farm animals including: cattle; horses; sheep; goats; and pigs, and household pets including: cats; and dogs, that have similar pathologies.

In other embodiments, the compositions and formulations of the present invention are administered to a human subject that has been diagnosed with a neurodegenerative disorder or suspected of having a neurodegenerative disorder. According to the present invention, treatment with a therapeutic of the invention encompasses the treatment of patients already diagnosed as having a neurodegenerative disorder at any clinical stage; the prevention of the disease in the patients with early symptoms and signs; the delay of the onset or evolution or aggravation or deterioration of the symptoms or signs of a neurodegenerative disorder; and/or promoting regression of a neurodegenerative disorder in symptomatic patients.

As an alternative to the nucleic acid and antibody approaches to SCA-1 therapy discussed below, useful SCA-1 therapeutics include small molecule agonists of SCA-1 enhancers and small molecule antagonists of ataxin-1 and/or SCA-1 suppressors. Methods of identification of small molecule therapeutics that are useful for this purpose are discussed in Section 5.14 below.

In yet other embodiments, the use of antibodies that bind to SCA-1 modifier gene encoded proteins for the treatment of neurodegenerative disorders is contemplated. Such antibodies can be agonists of SCA-1 enhancer gene products or antagonists of SCA-1 suppressor gene products. Methods of making such antibodies is described in Section 5.10.3, supra.

5.13.1. Repressing SCA-1 Modifier Genes

The invention also provides for antisense uses of SCA-1 modifier genes. In a specific embodiment, a SCA-1 modifier protein function is inhibited by use of SCA-1 modifier antisense nucleic acids. The present invention provides for use of nucleic acids of at least six nucleotides that are antisense to a gene or cDNA encoding an SCA-1 modifier protein or a portion thereof. A SCA-1 modifier “antisense” nucleic acid as used herein refers to a nucleic acid capable of hybridizing to a sequence-specific (i.e. non-poly A) portion of an SCA-1 modifier RNA (preferably mRNA) by virtue of some sequence complementarily. Antisense nucleic acids may also be referred to as inverse complement nucleic acids. The antisense nucleic acid may be complementary to a coding and/or noncoding region of an SCA-1 modifier mRNA. Such antisense nucleic acids have utility in inhibiting an SCA-1 modifier protein function.

The antisense nucleic acids can be oligonucleotides that are double-stranded or single-stranded, RNA or DNA or a modification or derivative thereof, which can be directly administered to a cell, or which can be produced intracellularly by transcription of exogenous introduced sequences. In a preferred embodiment, the antisense nucleic acids are double-stranded RNA mentioned previously (see Fire et al., 1998, Nature 391:806-811).

The SCA-1 modifier antisense nucleic acids are preferably oligonucleotides (ranging from 8 to about 50 oligonucleotides). In specific aspects, an oligonucleotide is at least 10 nucleotides, at least 15 nucleotides, at least 100 nucleotides, or at least 200 nucleotides in length. The oligonucleotide can be DNA or RNA or chimeric mixtures or derivatives or modified versions thereof, or single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. U.S.A. 84:648-652; PCT Publication No. WO 88/09810, published Dec. 15, 1988) or the blood-brain barrier (see e.g., PCT Publication No. WO 89/10134, published Apr. 25, 1988), hybridization-triggered cleavage agents (see e.g., Krol et al., 1988, BioTechniques 6:958-976) or intercalating agents (see e.g., Zon, 1988, Pharm. Res. 5:539-549).

The SCA-1 antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide may be conjugated to another molecule, e.g., a peptide, a hybridization-triggered cross-linking agent, a transport agent, a hybridization-triggered cleavage agent, etc.

Oligonucleotides may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (Stein et al., 1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, a SCA-1 modifier antisense oligonucleotide comprises catalytic RNA, or a ribozyme (see e.g., PCT Publication WO 90/11364, published Oct. 4, 1990; Sarver et al., 1990, Science 247:1222-1225). In another embodiment, the oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

In an alternative embodiment, the SCA-1 modifier antisense nucleic acid is produced intracellularly by transcription from an exogenous sequence. For example, a vector can be introduced in vivo such that it is taken up by a cell, within which cell the vector or a portion thereof is transcribed, producing an antisense nucleic acid (RNA). Such a vector would contain a sequence encoding the SCA-1 modifier antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the SCA-1 modifier antisense RNA can be by any promoter known in the art. Such promoters can be inducible or constitutive. Such promoters include but are not limited to: the SV40 early promoter region (Benoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids comprise a sequence complementary to at least a sequence-specific portion of an RNA transcript of a SCA-1 modifier gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded SCA-1 modifier antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a SCA-1 modifier RNA it may contain and still form a stable duplex (or triplex, as the case may be). One skilled in the art can ascertain a tolerable degree of mismatch by use of standard procedures to determine, e.g., the melting point of the hybridized complex.

Alternatively, endogenous SCA-1 modifier gene expression can be reduced by targeting deoxyribonucleotide sequences complementary to the regulatory region of the a SCA-1 modifier gene, including but not limited to a SCA-1 modifier gene promoter and/or enhancer, to form triple helical structures that prevent transcription of the SCA-1 modifier in target cells in the central nervous system (see generally, Helene, 1991, Anticancer Drug Des., 6(6), 569-584; Helene et al., 1992, Ann. N.Y. Acad. Sci., 660, 27-36; and Maher, 1992, Bioassays 14(12), 807-815).

5.13.2. Gene Therapy with SCA-1 Enhancers

With respect to increasing the level of expression or activity of a SCA-1 modifier gene, a SCA-1 modifier gene can be administered, for example, in the form of gene therapy. Gene therapy refers to therapy performed by the administration to a subject of an expressed or expressible nucleic acid. In this embodiment of the invention, the SCA-1 enhancer nucleic acids produce their encoded protein that mediates a therapeutic effect. Specifically, one or more copies of a normal SCA-1 modifier gene or a portion of a SCA-1 modifier gene that directs the production of a SCA-1 modifier gene product exhibiting normal SCA-modifier gene function, may be inserted into the appropriate cells within a patient, using any of the methods for gene therapy available in the art can be used according to the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the therapeutic comprises nucleic acid sequences encoding a SCA-1 enhancer, said nucleic acid sequences being part of expression vectors that express the SCA-1 enhancer or fragments or chimeric proteins or heavy or light chains thereof in a suitable host. In particular, such nucleic acid sequences have promoters operably linked to the SCA-1 enhancer coding region, said promoter being inducible or constitutive, and, optionally, tissue-specific. In another particular embodiment, nucleic acid molecules are used in which the SCA-1 enhancer coding sequences and any other desired sequences are flanked by regions that promote homologous recombination at a desired site in the genome, thus providing for intrachromosomal expression of the SCA-1 enhancer gene (Koller and Smithies, 1989, Proc. Natl. Acad. Sci. USA 86:8932-8935; Zijlstra et al., 1989, Nature 342:435-438.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22635; W092/20316; W093/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding a SCA-1 enhancer protein are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the SCA-1 enhancer to be used in gene therapy are cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoictic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a SCA-1 enhancer gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the SCA-1 enhancer gene is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. The amount of cells envisioned for use depends on the desired effect, patient state, etc., and can be determined by one skilled in the art.

In an embodiment in which recombinant cells are used in gene therapy, nucleic acid sequences encoding a SCA-1 enhancer are introduced into the cells such that they are expressible by the cells or their progeny, and the recombinant cells are then administered in vivo for therapeutic effect. In a specific embodiment, neural stem or progenitor cells are used. It was generally assumed that neurogenesis in the central nervous system ceases before or soon after birth. In recent years, several studies have presented evidence indicating that at least to some degree new neurons continue to be added to the brain of adult vertebrates (Alvarez-Buylla and Lois, 1995, Stem Cells (Dayt) 13:263-272). The precursors are generally located in the wall of the brain ventricles. It is thought that from these proliferative regions, neuronal precursors migrate towards target positions where the microenvironment induces them to differentiate. Studies have been reported where cells from the sub-ventricular zone can generate neurons both in vivo as well as in vitro, reviewed in Alvarez-Buylla and Lois, 1995, Stem Cells (Dayt) 13:263-272.

The neuronal precursors from the adult brain can be used as a source of cells for neuronal transplantation (Alvarez-Buylla, 1993, Proc. Natl. Acad. Sci. USA 90:2074-2077). Neural crest cells have also been long recognized to be pluripotent neuronal cells which can migrate and differentiate into different cell neuronal cell types according to the instructions they receive from the microenvironment they find themselves in (LeDouarin and Ziller, 1993, Curr. Opin. Cell Biol. 5:1036-1043).

5.14. Uses of SCA-1 Modifier Genes to Screen for Compounds with SCA-1 Activity

This invention also encompasses methods for identifying compounds that exhibit activity against neurodegenerative disorders, and in particular polyglutamine diseases such as SCA-1. More particularly, this invention encompasses the identification compounds that interact with components of cellular pathways that contribute to neurodegeneration, including but not limtied to SCA-1 neurodegeneration, as delineated by the modifier screens of the invention, and their use as therapeutics. Specifically, the invention encompasses the identification and use of agonists of SCA-1 enhancer genes and antagonists of SCA-1 suppressor genes in therapy of neurodegenerative disorders. Such compounds may bind to SCA-1 modifier genes or SCA-1 modifier gene products with differing affinities, and may serve as modifiers of the activity of SCA-1 modifier genes or SCA-1 modifier gene products in vivo with useful therapeutic applications in controlling the SCA-1 phenotype. The invention encompasses in vitro, in vivo, and cell-based screening methods to identify agonists of SCA-1 enhancer genes and antagonists of SCA-1 suppressor genes.

Additionally, the invention encompasses using the ataxin-1 transgenic animals of the invention to screen for compounds inhibit SCA-1 pathogenesis. Without limitation as to mechanism, such compounds may promote ataxin-1 clearance from cells or prevent its nuclear localization, thereby controlling SCA-1 pathogenesis.

5.14.1. In Vitro Screening Assays

The present invention provides in vitro screening assays for therapeutics for neurodegenerative disorders. In certain embodiments of the invention, compounds and compositions are tested for modulating effects on SCA-1 modifier gene products. In particular, compounds and compositions can be tested for modulating effects on stability, expression, and/or activity of the SCA-1 modifier gene products. In certain specific embodiments of the invention, test compounds are tested for agonist effects on a SCA-1 enhancer gene product. In certain other embodiments of the invention, test compounds are tested for antagonist effects on a SCA-1 suppressor gene product. Modulators of SCA-1 modifiers, i.e., agonists of SCA-1 enhancers and antagonists of SCA-1 suppressors, can be used as therapeutics for neurodegenerative disorders.

In certain embodiments, the screening assays are based on contacting a SCA-1 modifier protein with a test molecule and determining if the test molecule binds to the SCA-1 modifier protein. If the test molecule binds to the SCA-1 modifier protein, the test molecule can be assayed for agonist or antagonist effects on the SCA-1 modifier protein. In one non-limiting example, the SCA-1 modifier protein is labeled and used to contact a peptide λg11 expression library to identify a peptide molecule to which the SCA-1 modifier binds.

In another embodiment, the screening assays are based on the ability of a test molecule to agonize or antagonize the function of a SCA-1 modifier protein, taking into account the nature of the function of the SCA-1 modifier gene and its encoded protein. For example, if a SCA-1 modifier gene encodes an RNA binding protein (such as pumilio or mushroom-body expressed), in vitro-formed complexes of SCA-1 modifier proteins and their RNA targets can be contacted with test molecules to identify molecules the inhibit the interaction. Alternatively, the RNA target sites of the SCA-1 modifier proteins can be contacted with test molecules to identify molecules that bind to the RNA target sites and in doing so mimic binding of the SCA-1 modifier protein to the target site.

In vitro systems can be designed to identify compounds capable of binding the SCA-1 modifier gene products. Compounds identified can be useful, for example, in modulating the activity of wild type and/or mutant SCA-1 modifier gene products, can be utilized in screens for identifying compounds that disrupt normal interactions of SCA-1 modifier gene products, or can in themselves disrupt such interactions.

The principle of the assays used to identify compounds that bind to the SCA-1 modifier gene products involves preparing a reaction mixture of a SCA-1 modifier gene product and a test compound under conditions and for a time sufficient to allow the two components to interact and bind, thus forming a complex which can be removed and/or detected in the reaction mixture. These assays can be conducted in a variety of ways. For example, one method to conduct such an assay involves anchoring a particular SCA-1 modifier gene product or the test substance onto a solid phase and detecting SCA-1 modifier gene product/test compound complexes anchored on the solid phase at the end of the reaction. In one embodiment of such a method, a SCA-1 modifier gene product can be anchored onto a solid surface, and the test compound, which is not anchored, can be labeled, either directly or indirectly.

In practice, microtiter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific to the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific to the SCA-1 modifiers or the test compound to anchor any complexes formed in solution, and a labeled antibody specific for the other component of the possible complex to detect anchored complexes.

As an example, and not by way of limitation, techniques such as those described in this section can be utilized to identify compounds which bind to SCA-1 modifier gene products. For example, a SCA-1 modifier gene product can be contacted with a compound for a time sufficient to form a SCA-1 modifier gene product/compound complex and then such a complex can be detected.

Alternatively, the compound can be contacted with a SCA-1 modifier gene product in a reaction mixture for a time sufficient to form a SCA-1 modifier gene product/compound complex, and then such a complex can be separated from the reaction mixture.

In certain embodiments of the invention, the biochemical activity of the SCA-1 modifier gene products is determined by sequence alignment and comparison. It is well-known to the skilled artisan that certain biochemical activities of a protein can be determined based on its amino acid sequence. Based on this information, biochemical assays can be designed, and compounds and compositions can be tested for modulating effects on the activity of a SCA-1 modifier gene product in this assay. As an example, and not by way of limitation, the SCA-1 modifier gene product of interest can be a kinase. In order to conduct the assay, the SCA-1 modifier gene products is incubated with an appropriate substrate and ATP under reaction conditions, and for a time sufficient for the phosphorylation of the substrate by the kinase. For the quantification of the kinase reaction, e.g., radioactively labeled ATP can be used. Subsequent to the incubation, the reaction mixture is resolved by SDS PAGE, and the gel is subsequently exposed to an x-ray film to detect the incorporated radioactivity. The intensity of the signal is proportional to the kinase activity of the SCA-1 modifier gene product. In order to identify modulators of the SCA-1 modifier gene product, different compounds and compositions are added to the reaction mixture and their effect on the kinase activity is determined.

Among the SCA-1 modifiers which can be utilized for such methods are, for example, the genes listed in Tables 2-4 of the application, and naturally occurring variants thereof.

The term “naturally occurring variant,” as used herein refers to an amino acid sequence homologous to the SCA-1 modifier gene products in Drosophila or in a different species, such as, for example, an allelic variant of a SCA-1 modifier which maps to the same chromosomal location as the nucleotide sequence encoding the SCA-1 modifier gene product, or a location syntenic to such a location. Among the allelic variants which can be utilized herein are allelic variant sequences encoded by a nucleotide sequence that hybridizes under stringent conditions to the complement of a nucleotide sequence encoding the SCA-1 modifier gene products described hereinabove.

As an alternative, or in addition, to the in vitro methods discussed above, computer modelling and searching technologies can be used to identify compounds, or improve already identified compounds, that can modulate SCA-1 modifier expression or activity. Having identified such a compound or composition, the active sites or regions are preferably identified.

The three dimensional geometric structure of the active site is then preferably determined. This can be done by known methods, including X-ray crystallography, which can determine a complete molecular structure. Solid or liquid phase NMR can also be used to determine certain intra-molecular distances within the active site and/or in the ligand binding complex. Any other experimental method of structure determination can be used to obtain partial or complete geometric structures.

Methods of computer based numerical modelling can be used to complete the structure (e.g., in embodiments wherein an incomplete or insufficiently accurate structure is determined) or to improve its accuracy. Any art recognized modelling method may be used, including, but not limited to, parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. Exemplary forcefields that are known in the art and can be used in such methods include, but are not limited to, the Constant Valence Force Field (CVFF), the AMBER force field and the CHARM force field. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential target or pathway gene product modulating compounds.

Alternatively, these methods can be used to identify improved modulating compounds from an already known modulating compound or interacting protein. The composition of the known compound can be modified and the structural effects of modification can be determined using the experimental and computer modelling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Examples of molecular modelling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modelling of drugs interactive with specific proteins, such as Rotivinen et al., 1988, Acta Pharmaceutical Fennica 97:159-166; Ripka, (Jun. 16, 1988), New Scientist 54-57; McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 1-162; and, with respect to a model receptor for nucleic acid components, Askew et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

5.14.2. Cell-Based Screening Assays

The present invention additionally provides cell based screening assays for SCA-1 therapeutics for those SCA-1 modifiers whose activities are known. These assays can be used in primary screens with compound libraries or as confirmatory assays for molecules that are identified to bind in vitro to a SCA-1 modifier protein. The particular cell culture assay will depend on the function of the SCA-1 modifier, since as described in Section 6.12, infra, SCA-1 modifier genes have a variety of different functions.

In one embodiment in which a SCA-1 modifier gene encodes a transcriptional regulator, such as the Sin3A or Rpd3 transcriptional repressors, a reporter gene assay can be used to monitor activity of the SCA-1 modifier. Such assays entail operatively linking the binding site of the transcriptional regulator (or the binding site of a complex in which the transcriptional regulator is a component) to a reporter gene, and monitoring gene expression upon contacting cells with test molecules. Reporter gene activation or repression may be monitored, depending on the nature of the transcriptional regulation provided by the SCA-1 modifier protein and whether an agonist or antagonist of the SCA-1 modifier protein is sought.

Many of the proteins encoded by the SCA-1 modifier genes are parts of multiprotein complexes. Screening assays can be designed to identify molecules that inhibit or enhance the interaction of the SCA-1 modifier protein with other components of the multiprotein complexes. In one non-limiting example, the SCA-1 modifier protein and its interaction partner are used in a yeast two-hybrid system. The SCA-1 modifier protein and its interaction partner are each expressed either as a fusion protein with a transcriptional activation domain and a transcriptional DNA binding domain in yeast strain containing a reporter gene that is responsive to the DNA binding domain fused to the SCA-1 modifier protein or its interaction partner. Colonies of the yeast which express the two fusion proteins and the reporter are contacted with test molecules to identify molecules that reduce or increase the interaction between the SCA-1 modifier protein and its interaction partner, as measured by the levels of reporter gene expression.

In yet another embodiment, PC12 rat pheochromocytoma cells (Greene et al., 1991, “Methodologies for the culture and experimental use of the rat PC12 rat pheochromocytoma cells line,” pp. 207-225, In: Culturing Nerve Cells, The MIT Press, Cambridge, Mass.) are utilized as a cell culture model for a neurodegenerative disorder. Specifically, survival of and neurite outgrowth from differentiated PC12 can be assayed to identify agents for the treatment of neurodegenerative disorders.

In certain modes of the embodiment, activity of one or more SCA-1 enhancer genes in PC12 cells is disrupted (for example through antisense expression or ribozymes), which would be expected to reduce survival of differentiated PC12 cells and reduce neurite outgrowth from the cells. The cells can then be contacted with a variety of test compounds, and cell survival or neurite outgrowth phenotypes scored. A compound which increases the survival of the PC12 cells or neurite outgrowth from the PC12 cells is a candidate therapeutic for a neurodegenerative disorder.

Conversely, a SCA-1 suppressor gene can be overexpressed in PC12 cells, which would be expected to reduce survival of differentiated PC12 cells and reduce neurite outgrowth from the cells. The cells can then be contacted with a variety of test compounds, and cell survival or neurite outgrowth phenotypes scored. A compound which increases the survival of the PC12 cells or neurite outgrowth from the PC12 cells is a candidate therapeutic for a neurodegenerative disorder.

5.14.3. IN VIVO Screening Assays

The present invention fulrther provides in vivo screening assays for SCA-1 therapeutics that are based on contacting Drosophila cultures with a SCA-1 phenotype or with a propensity to develop a SCA-1 phenotype, with a test molecule, and determining if the test molecule reduces or prevents SCA-1 pathogenesis.

In a preferred embodiment, assays can be performed to screen molecules that prevent SCA-1 pathogenesis by contacting a transgenic Drosophila line containing normal ataxin-1 (e.g., ataxin-1 30Q) or ataxin-1 with expanded polyglutamine repeats (e.g., ataxin-1 82Q) is with one or more test compounds, for example by applying the test compounds to the Drosophila culture media, and determining whether the progressive neuronal degeneration in animal is less severe than the progressive neuronal degeneration of a counterpart animal which expresses the same ataxin-1 transgene, and is preferably from the same transgenic line, but is not contacted with the test molecule.

In a highly preferred embodiment, the ataxin-1 transgene is expressed in the eye tissue of the animals, giving rise to a rough eye phenotype. In other embodiments, different manifestations of SCA-1 can be analyzed, such as, but not limited to, neural degeneration and nuclear inclusion formation. In a preferred embodiment, the neural degeneration phenotype against which test compounds are screened is a locomotor dysfunction. In another preferred embodiment, the neural degeneration phenotype is a reduced life span. The Drosophila life span can be reduced by 10-80%, e.g., approximately, 30%, 40%, 50%, 60%, or 70%, by manipulating the expression levels of ataxin-1, for example as discussed in Section 5.3, supra.

The test compound can be applied at different stages of Drosophila development. Preferably, the test compound is added to the Drosophila culture during the larval stages, most preferably at the third larval instar stage, which is the main larval stage in which eye development takes place.

The test compound can be fed to the Drosophila at different stages of their development and to adult Drosophila. In one embodiment, the test compound is mixed in to Drosophila food, most preferably the yeast paste that can added to Drosophila cultures.

Screening assays analogous to those described for Drosophila misexpressing ataxin-1 can be done for Drosophila that misexpress a SCA-1 suppressor gene or Drosophila that are mutant for a SCA-1 enhancer gene, and are encompassed by the present invention.

In the in vivo screening methods of the invention, a library of test compounds can be applied to filter strips, which are then placed individually in the Drosophila culture vials, for screening.

In a specific embodiment of the invention, compounds from a compound library are administered by microinjection, preferably by microinjection, into Drosophila hemolymph, as described in WO 00/37938, published Jun. 29, 2000.

For efficiency of screening, and in addition to screening with individual test compounds, test compounds can be administered in pools of at least 5, 10, 20, 50, or 100 compounds. Upon identifying a “hit”, i.e., a modifier of a phenotype associated with ataxin-1 or a SCA-1 modifier gene, the individual components of the pool can be assayed independently to identify the particular compound of interest.

The screening assays, described herein, can be used to identify compounds and compositions, including peptides and organic, non-protein molecules that can suppress SCA-1 pathogenesis in transgenic Drosophila expressing normal ataxin-1 or ataxin-1 with expanded glutamine repeats. Recombinant, synthetic, and otherwise exogenous compounds may have activity and, therefore, may be candidates for pharmaceutical agents.

5.14.4. Behavioral Assays

In one embodiment of the in vivo screening assays of the invention, test compounds can be assayed for their abilities to modify behavioral deficits produced in flies as a result of misexpressing vertebrate disease genes in the central nervous system of Drosophila. In a preferred embodiment, the vertebrate disease gene is a mammalian disease gene, most preferably a human disease gene. Neuronal degeneration in the central nervous system will give rise to behavioral deficits, including but not limited to motor deficits, that can be assayed and quantitated in both larvae and adult Drosophila. For example, failure of Drosophila adult animals to climb in a standard climbing assay (see, e.g., Ganetzky and Flannagan, 1978, J. Exp. Gerontology 13:189-196; LeBourg and Lints, 1992, J. Gerontology 28:59-64) is quantifiable, and indicative of the degree to which the animals have a motor deficit and neurodegeneration. Other aspects of Drosophila behavior that can be assayed include but are not limited to circadian behavioral rhythms, feeding behaviors, habituation to external stimuli, and odorant conditioning.

Screening for a therapeutic of the vertebrate disease caused by expression of a related vertebrate disease gene in the Drosophila central nervous system can be achieved by contacting larvae or adult flies with a climbing behavior deficit caused by the expression of the vertebrate disease gene with test compounds, as described above, and identifying a molecule that reduces the abnormal climbing behavior of the animals.

In additional to the neurodegenerative disorders described herein, the disclosed methods can be used to screen for a modifier of other vertebrate diseases such as proliferative disorders, skeletal muscle disorders, pancreatic disorders, heart and cardiovascular disorders, pulmonary (lung) disorders, pituitary related disorders, adrenal disorders, thyroid gland disorders, gastric, intestinal and colonic disorders, hepatic (liver) disorders, renal (kidney) disorders, spleen disorders, bone disorders, bone marrow disorders, eye disorders, prostate disorders, leukocytic disorders, such as leukopenias (e.g., neutropenia, monocytopenia, lymphopenia, and granulocytopenia), immune disorders, inflammatory disorders, apoptotic disorders, and immune disorders.

In a particular embodiment, the proliferative disorder is cancer. Suitable cancers are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, or heavy chain disease. In another particular embodiment, the proliferative disorder is a skeletal muscle disorder, including but not limited to a muscular dystrophy, a motor neuron disease, or a myopathy.

5.14.5. Sources of Test Compounds

The screening assays described herein may be used to identify small molecules, peptides or proteins, or derivatives, analogs and fragments thereof, that candidate therapeutics for SCA-1.

Compounds that may be useful in the screening assays of the inventions include but are not limited to peptides derived from a random peptide library as well as combinatorial chemistry-derived molecular library made of D-and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

In one embodiment of the present invention, peptide libraries may be used as a source of test compounds that can be used to screen for SCA-1 therapeutics. Diversity libraries, such as random or combinatorial peptide or nonpeptide libraries can be screened for molecules that specifically modify the SCA-1 phenotype. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al, 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lemer, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian et al, 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been pernethylated to generate a chemically transformed combinatorial library, is described by Ostresh et al. (1994, Proc. Natl. Acad. Sci. USA 91:11138-11142).

Compounds that can be tested and identified methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, Germany), Takasago (Rockleigh, N.J.), SST Corporation (Clifton, N.J.), Ferro (Zachary, La. 70791), Riedel-deHaen Aktiengesellschaft (Seelze, Germany), PPG Industries Inc., Fine Chemicals (Pittsburgh, Pa. 15272). Further any kind of natural products may be screened using the methods described herein, including microbial, fungal, plant or animal extracts.

Furthermore, diversity libraries of test compounds, including small molecule test compounds, may be utilized. For example, libraries may be commercially obtained from Specs and BioSpecs B. V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilized, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other approaches are applicable to peptide, non-peptide oligomer or small molecule libraries of compounds (Lam, 1997, Anticancer Drug Des.12:145). Combinatorial libraries of test compounds, including small molecule test compounds, can be utilized, and may, for example, be generated as disclosed in Eichler & Houghten, 1995, Mol. Med. Today 1:174-180; Dolle, 1997, Mol. Divers. 2:223-236; and Lam, 1997, Anticancer Drug Des. 12:145-167.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

5.14.6. Drug Development

The SCA-1 modifiers described herein are prime targets for SCA-1 therapeutic drugs, including but not limited to small molecule therapeutics. Thus, the present invention encompasses the use of SCA-1 modifiers identified by the methods described herein in drug validation studies. Methods are provided for determining whether a given SCA-1 modifier is a target of a SCA-1 therapeutic. Such methods entail comparing the effect of a drug on an ataxin-1 misexpressing animal to the drug's effect on animal that misexpresses ataxin-1 but also harbors a mutation in a SCA-1 modifier. As will be apparent to one of skill in the art and as will be discussed below, such comparative studies allow the validation of drug targets, and where desired, such methods can be exploited to screen for a SCA-1 therapeutic that targets a specific SCA-1 modifier if so desired.

The comparative screening methods of the present invention are premised on the principle that altering the expression levels or activity of a SCA-1 modifier will modulate the toxicity of ataxin-1. For example, where the SCA-1 modifier is a SCA-1 enhancer gene, increasing the expression or activity of the SCA-1 modifier will ameliorate the toxicity of ataxin-1 expression. If a SCA-1 therapeutic targets the SCA-1 enhancer gene product, then overexpression of the enhancer gene product will titrate out the effect of the drug. Under such circumstances, the drug will have less of an effect—as assayed by any of the phenotypes discussed in Section 5.14.3 and 5.14.4, supra, including but not limited to behavioral and life span assays—than would otherwise be expected. Conversely, if a SCA-1 therapeutic targets a SCA-1 suppressor gene, reducing the dosage of the suppressor gene (e.g., by introducing a heterozygous loss of function mutation) in ataxin-1 misexpressing Drosophila will sensitize the animals to the drug and accordingly, the drug will have a greater effect than is otherwise expected. As described extensively in Sections 5.14.3 and 5.14.4, the effects of the drugs can be assayed with respect to locomotor deficits, reduced life span, rough eyes, etc.

Additionally, once a SCA-1 therapeutic, e.g., small molecule, is identified, its can be assayed for its therapeutic effects on other neurodegenerative disorders. In a preferred embodiment, the other neurodegenerative disorders are polyglutamine disorders, including but not limited to spinocerebellar ataxia (SCA)-1, SCA-2, SCA-6, SCA-7, Machado-Joseph disease (MJD), Huntington Disease (HD), spinobulbar muscular atrophy (SBMA), or dentatorubropallidolusyan atrophy (DRPLA).

5.15. Formulations

Pharmaceutical compositions for use in accordance with the present invention may be formulated in conventional manner using one or more physiologically acceptable carriers or excipients.

Thus, the therapeutics of the invention (antagonists of ataxin-1, agonists of SCA-1 enhancers and antagonists of SCA-1 suppressors) of the invention and physiologically acceptable salts and solvates thereof may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate) lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the therapeutics of the invention for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The therapeutics of the invention may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The therapeutics of the invention may also be formulated in rectal compositions such as suppositories or retention enemas, e.g., containing conventional suppository bases such as cocoa butter or other glycerides.

In addition to the formulations described previously, the therapeutics of the invention may also be formulated as a depot preparation. Such long acting formulations may be administered by implantation (for example subcutaneously or intramuscularly) or by intramuscular injection. Thus, for example, the therapeutics of the invention may be formulated with suitable polymeric or hydrophobic materials (for example as an emulsion in an acceptable oil) or ion exchange resins, or as sparingly soluble derivatives, for example, as a sparingly soluble salt.

In certain embodiments of the invention, the therapeutics of the invention are the pharmaceutically acceptable carrier is not water.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration preferably for administration to a human.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further described in the following examples which are in no way intended to limit the scope of the invention.

### 6. EXAMPLES

### Transgenic Drosophila for Polyglutamine-Induced Neurodegenerative Disorders

6.1. Materials and Methods

6.1.1. Drosophila Genetics

Fly culture and crosses were at 23° C. unless otherwise noted. The UAS: SCA-1 30Q and UAS: SCA-1 82Q transgenic flies were generated by cloning two human SCA-1 cDNAs containing 30 and 82 CAG repeats respectively (Burright et al., 1995, Cell 82:937-48) in the pUAST transformation vector (Brand and Perrinon, 1993, Development 118:401-415). These constructs were injected in a y¹w¹¹¹⁸ strain as described (Rubin and Spradling, 1982, Science 218:348-353). The EP strains were provided by C. Cater and G. Rubin. hsc70-4¹⁹⁵ flies were provided by S. Artavanis-Tsakonas. cpo¹ and cpo^{L1} were provided by H. Bellen. pap⁵³ and pap^{EPI} were provided by D. Cribbs. The P strains and all other strains were provided by the Bloomington Drosophila Stock Center. For the modifier screens, females of the genotype y¹w¹¹¹⁸ UAS: SCA-1 82Q[F7]; gmr-GAL4/CyO were crossed to males from the collection of lethal P insertions, the collection of EP insertions or to other selected strains. Culture and crosses of the genetic screens were made at 25° C. Strains producing a modification of the 82Q[F7] characteristic phenotype were kept and crossed again to verify the interaction at 23° C., 27° C. and 29° C. EP lines were tested for specificity by crossing females of selected EP lines with males bearing y¹w¹¹¹⁸ UAS: SCAJ82Q[F7]; gmr-GAL4/CyO. Since UAS: SCAJ82Q[F7] is on the X chromosome, F1 males do not carry this transgene and serve as controls of EP specificity.

To produce the genotypes in FIG. 3 and Table 1 the crosses of UAS: tauGFP; ap^{VNC}/CyO flies with UAS: SCAJ82Q[M6] flies or UAS: LacZ (controls) were performed. Adult flies were aged in standard medium after eclosion.

6.1.2. Histology and Immunofluorescence

SEM images: whole flies were dehydrated in ethanol, critical point dried and analyzed with a JEOL JSM 6100 microscope. Sections of adult fly eyes: adult heads (0-1 day-old) were fixed 30 mm in 4% formaldehyde, washed, fixed for 3 hr at 4° C. in 1% osmium tetroxide, dehydrated in ethanol, embedded in Epon for vertical semi-thin sections, and then stained with toluidene blue. SCA-1-30Q and SCA-1-82Q mice are described in Burright et al., 1995, Cell 82:937-48. Mouse cerebellar sections were prepared as previously described (Cummings et al., 1999, Neuron 24:879-92) and stained with an anti-calbindin monoclonal antibody (1:1000, CL300 Sigma).

Eye imaginal discs and salivary glands were dissected in 1×PBS, fixed for 20 min in 4% formaldehyde, washed with 1×PBS, 0.1% TritonX-100, and incubated with the primary antibody. Adult ventral ganglions were prepared by fixing the whole fly thorax for 3 hours at 4° C. in 4% formaldehyde. After washing, the ventral ganglions were dissected and fixed again for 20 min at room temperature, and then stained as imaginal discs. The following primary antibodies were used: rabbit anti-ataxin-1 (11NQ, diluted 1:750; Skinner et al., 1997, Nature 389:97 1-4), mouse anti-laminin A (T47, 1:50; Harel et al., 1989, J Cell Sci 94:463-70), mouse anti-hsp70/hsc70 (5PA822, 1:100; StressGen), mouse anti-ubiquitin (Ubi-1, 1×; ZYMIED), mouse anti-195 Regulator ATPase subunit 6b (Thp7) (PW8175, 1:100; AFFINITI). The following secondary fluorochrome-conjugated antibodies were used: Alexa⁴⁸⁸ (1:400, Molecular Probe) and Cy3 (1:500, Jackson). Confocal microscopy was carried out on Bio-Rad MRC-1024 confocal imaging system, and images were collected with Lasersharp 3.0 software (Bio Rad) and modified with Adobe Photoshop 4.0 (Adobe).

6.1.3. Comparison of Ataxin-1 Immunoreactivity in Different SCA-1 Transgenic Lines

Eye imaginal discs to be compared, differing in genotype or temperature of culture, were manipulated in parallel as follows: First they were dissected in 1×PBS solution, fixed with 4% formaldehyde, incubated in rabbit anti-ataxin-1 antiserum 11NQ (diluted 1:750, Skinner et al., 1997, Nature 389:97 1-4) for 14 hr at 4° C., washed in PBT (1×PBS, 0.1% TritonX-100), and incubated in goat anti-rabbit antibody conjugated with Alexa⁴⁸⁸ (1:400, Molecular Probe). The eye imaginal discs were then mounted in Vectashield (Vector) and stored at −20° C. until viewing. An image series of 40 focal planes (total thickness 16 μM) was collected from the central region of each imaginal disc (384×384 pixel) with an Applied Precision Restoration Microscopy Optical Workstation (Applied Precision, Inc., Issaquah, Wash.). Optimal exposures were determined empirically to obtain images that did not contain any saturated pixels (i.e., less than 4095), and this exposure was used for all samples. Z-stacks (merged images) were then created by a constrained iterative algorithm using SoftWorx software (Applied Precision, Inc.). The total value of all pixels in the region was calculated by the software. To compare the values of different geriotypes or temperature conditions, relative percentages shown in figures were calculated by dividing the higher value with the lower value in each data set.

6.1.4. Analysis of P/EP Elements

Genomic DNA regions flanking the P and EP-elements were recovered from modifier lines by standard plasmid rescue and/or inverse PCR protocols (http://www.fruitfly.org). Recovered DNA was sequenced and analyzed with BLAST and GENEFINDER (http://dot.imgen.bcm.tmc.edu:933 1/gene-finder) in BDGP (http://www.fruitfly.org) and NCBI (http://www.ncbi.nlm.nih.govfBLAST) to confirm identity of the PIEP elements and to identify the modifier gene.

To verify specific gene overexpression in EP elements, coding sequences downstream of the EP region were obtained by PCR and used as probes for in situ hybridization (Mangiarini et al., 1996, Cell 87:493-506) in larvae carrying the dppGal4 driver and the EP-insertion of interest.

### 6.2. EXAMPLE

### Increased Expression of Wild-Type Human Ataxin-1 30Q Causes Neurodegenerative Phenotypes Resembling those Caused by Expanded Ataxin-1 82 Q

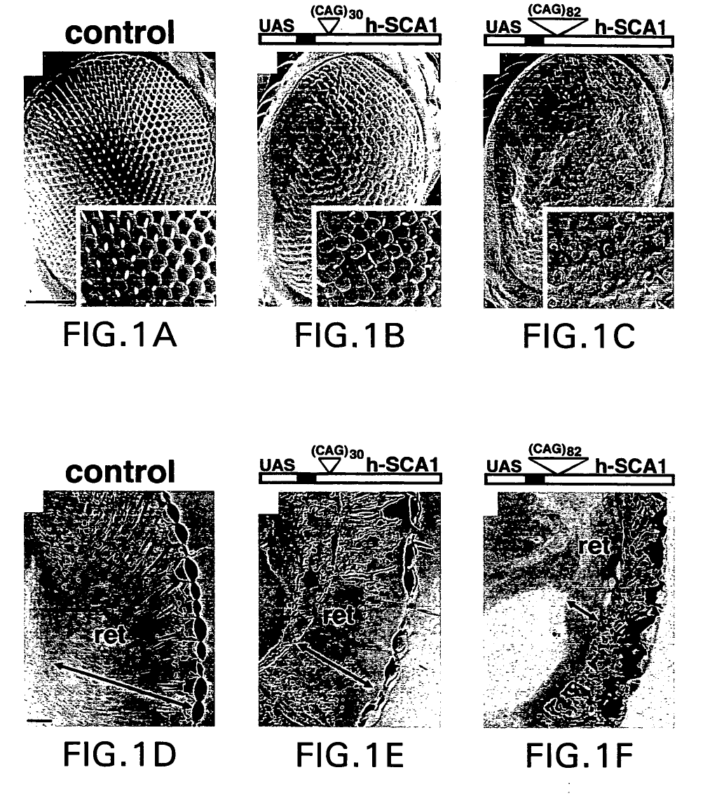

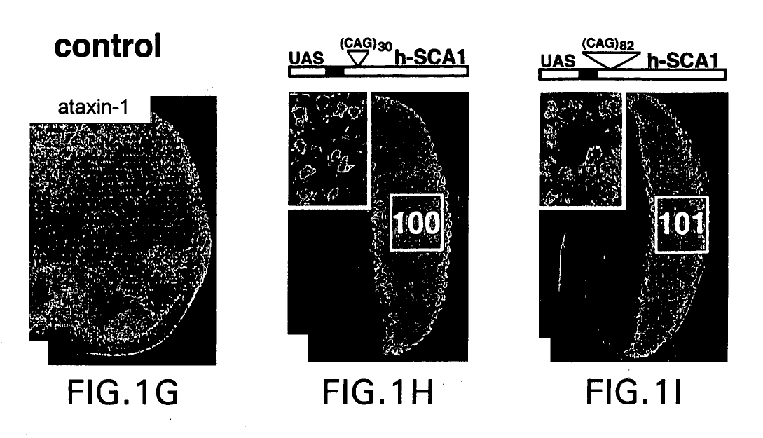

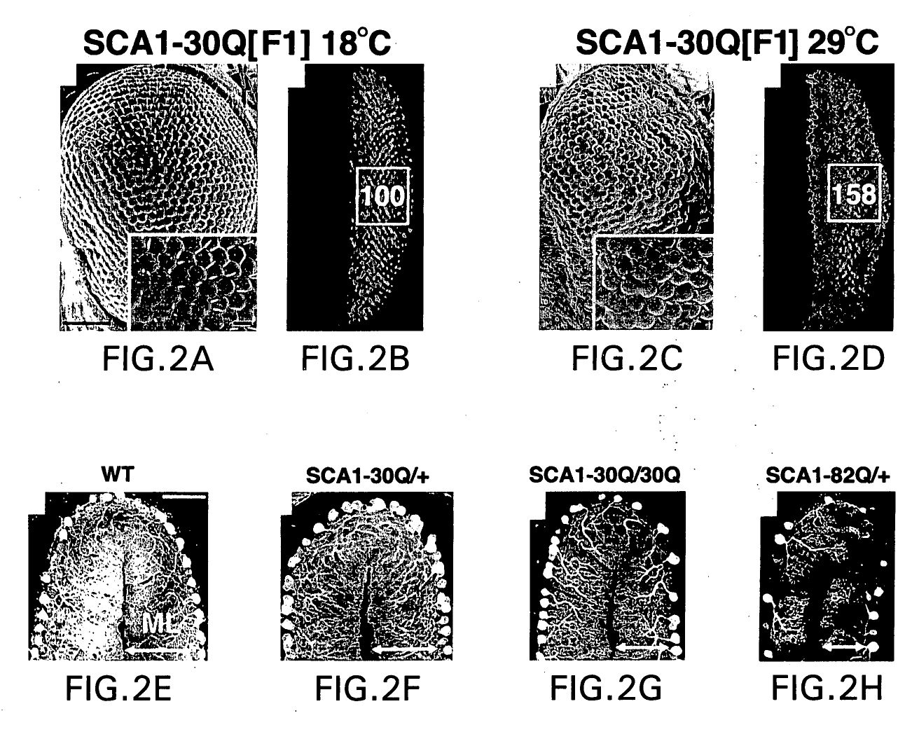

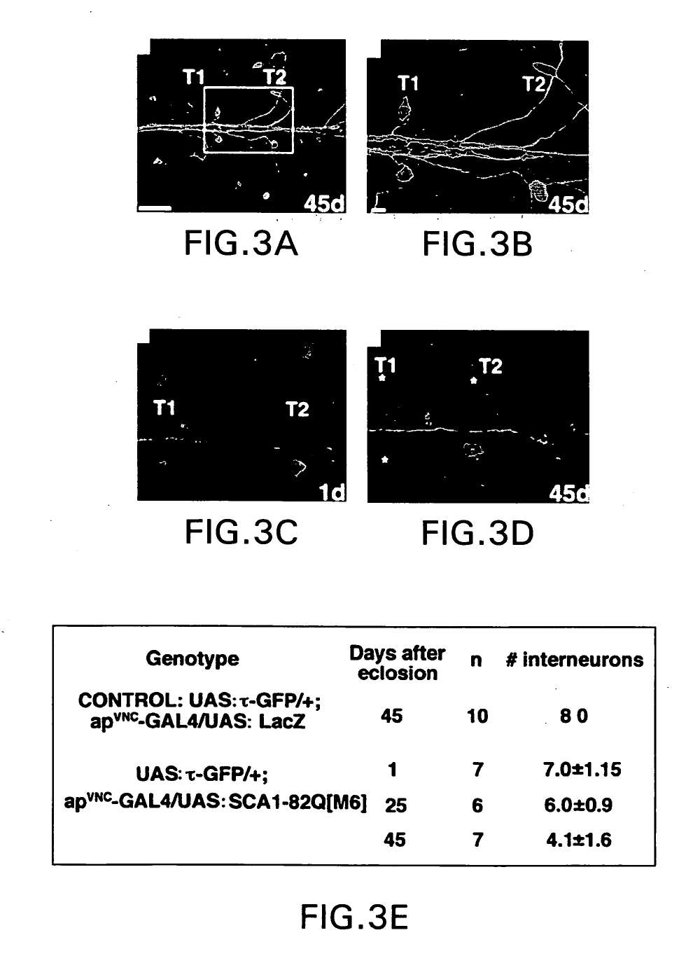

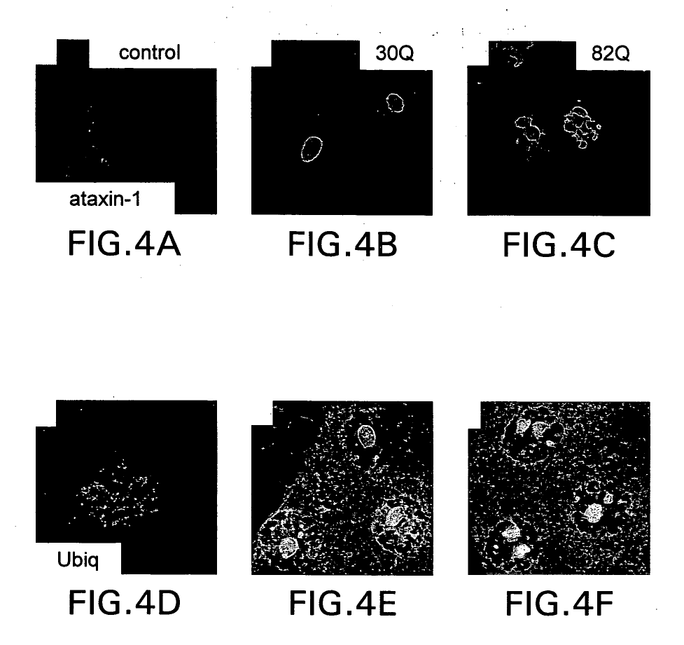

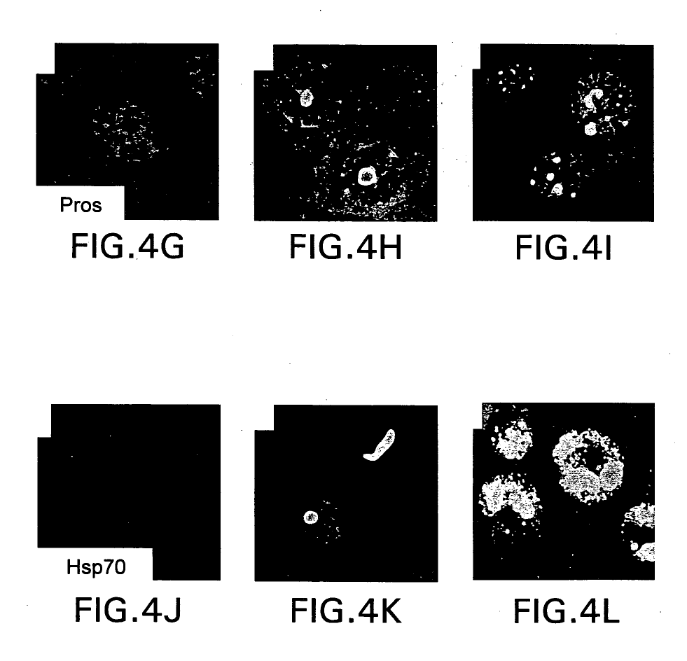

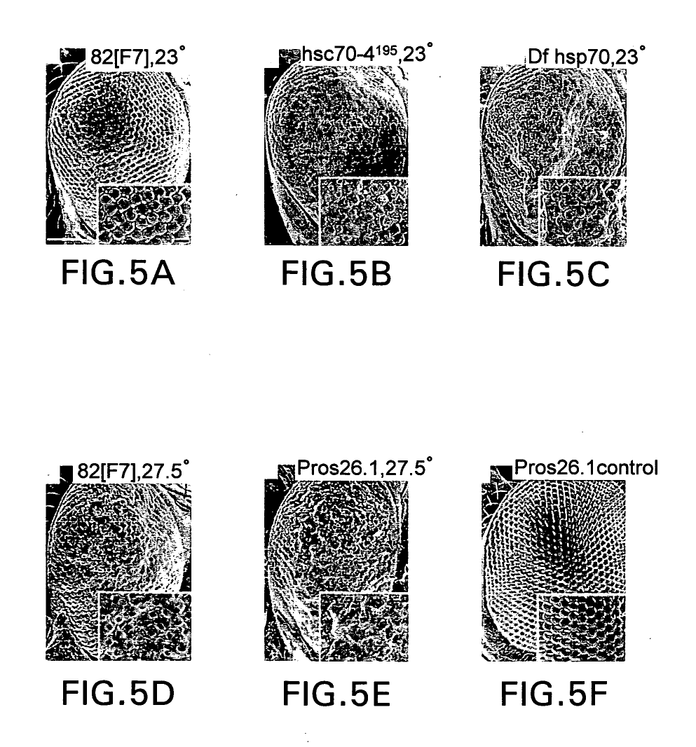

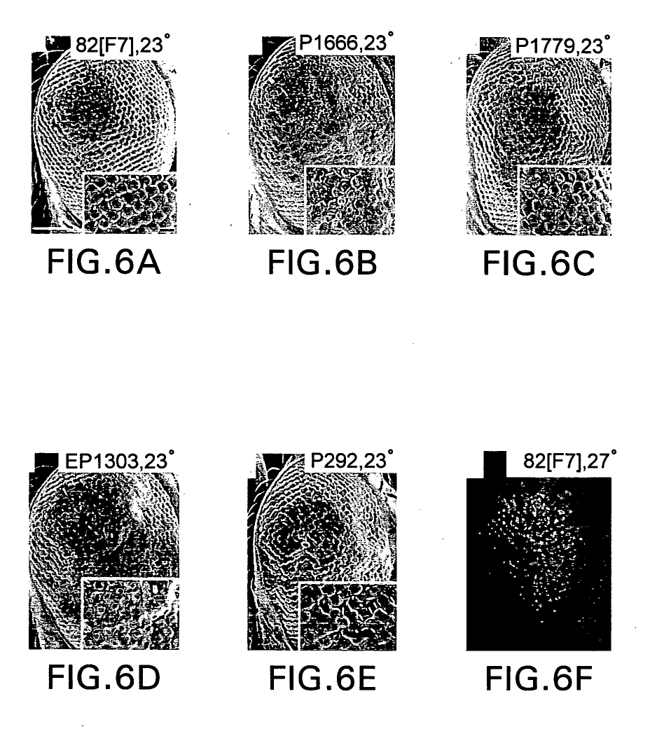

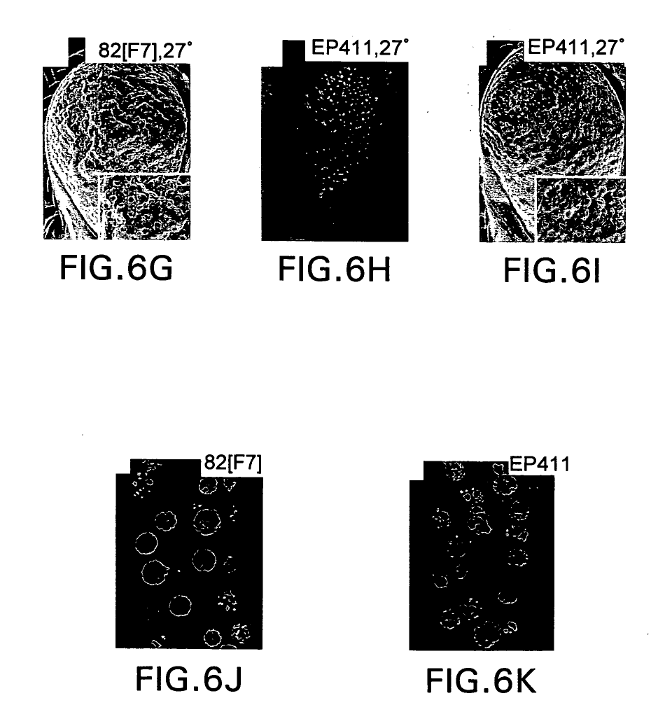

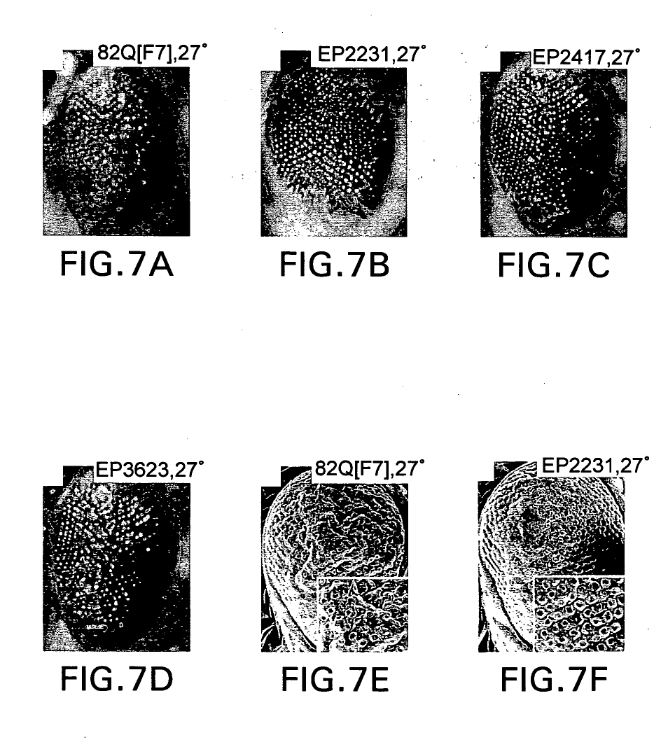

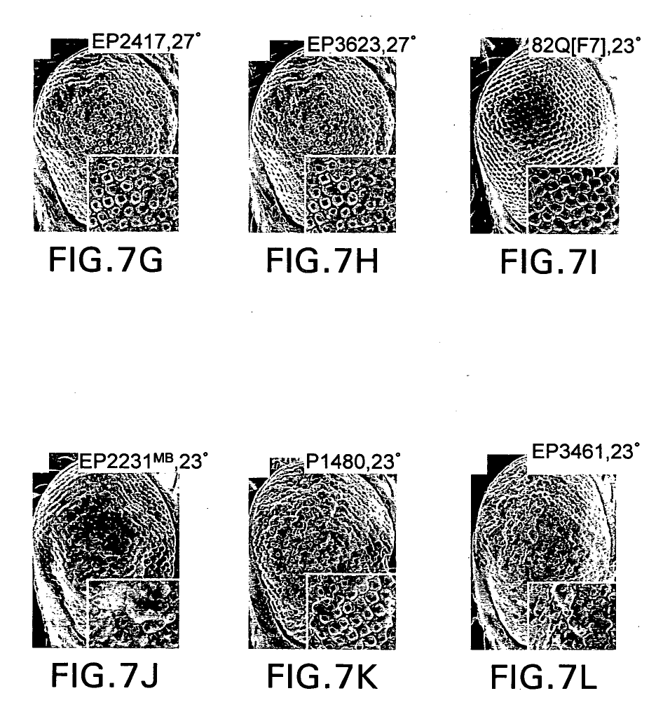

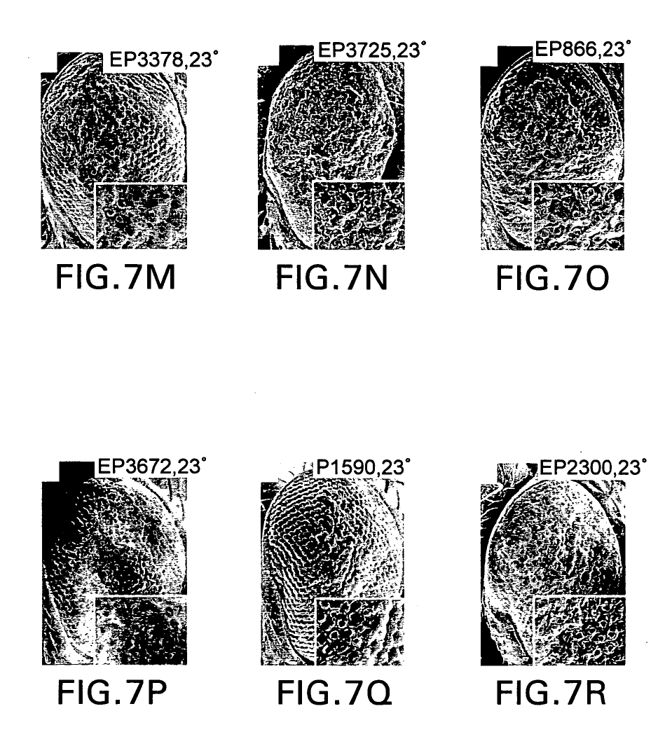

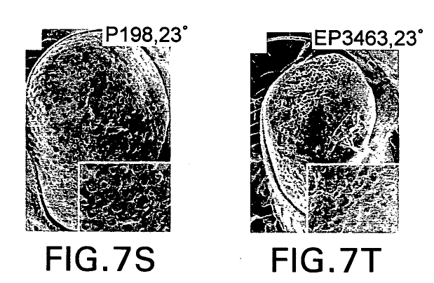

The GAL4/UAS system was used to induce expression of the SCA-1 constructs in transgenic flies. Two human SCA-1 cDNAs, differing in the size of their polyglutamine repeat tracts, were cloned into the Drosophila transformation vector pUAST. These constructs encoded ataxin-1 30Q (a wild-type human isoform) and ataxin-1 82Q (an expanded isoform) (see top of FIG. 1A). Six 82Q and four 30Q lines were generated.

Ataxin-1 expression was directed to the eye retina using the grnr-GAL4 driver (Moses and Rubin, 1991, Genes Dev 5:583-93). The eye is a sensitive system for investigating a variety of genetic pathways (Dickson and Hafen, Vol. II (eds. Bate, M. and Martinez Arias, A.) 1327-1362 (Cold Spring Harbor Laboratory Press, New York, 1993); Wolff et al., (eds. Cowan, T. M., Jessell, T. M. and Zypursky, S. L.) 474-508 (Oxford University Press, New York, 1997), and it is ideal for comparing the effects of SCA-1 transgenes with different polyglutamine tract lengths. Differing expression levels in various transgenic lines can cause phenotypes to vary considerably, so we estimated ataxin-1 levels in all transgenic lines by immnunofluorescence (see Methods). The levels of ataxin-1 cannot be quantified precisely by western analysis because only a fraction of the protein is recovered: ataxin-1 NI are insoluble in hot SDS, and the aggregates resist complete extraction and entry into gels (Koshy et al, (eds. Wells, R. D. and Warren, S. T.) 241-248 (Academic Press, San Diego, 1998) and confirmed by the inventors' own observations). The severity of the phenotype correlated well with expression levels within both the 82Q and the 30Q transgenic lines, although, as expected, the 82Q lines invariably showed a much stronger phenotype. The most severe eye phenotypes of the 82Q and 30Q lines are shown in FIG. 1A-F. Note that these two lines had roughly similar expression levels (FIG. 1H-I), but the phenotype of the 82Q line was much more severe.

Increasing the expression of ataxin-1 30Q by increasing the culture temperature led to a more pronounced phenotype (FIG. 2A-D). Similar results were observed when the dosage of the transgene was doubled (not shown). The finding that relatively high expression of wild-type human SCA-1 transgenes led to phenotypes similar to those produced by expanded alleles was unexpected. Previous analysis of transgenic mice that expressed the wild-type human SCA-1 30Q allele revealed no obvious pathology (Burright et al., 1995, Cell 82:937-48), but the expression level of the SCAJ 30Q A02 transgene was roughly one-quarter of the expression level of the SCA-1 82Q B05 transgene by northern analysis (Kiement et al. , 1998, Cell 95:41-53). To determine whether SCAJ 30Q could also cause neurodegeneration in mice, the cerebellar cortex of homozygous SCAJ 30Q A02 mice was examined. As shown in FIG. 2E-H, the dendritic arborization of the Purkinje cell layer was clearly abnormal at 59 weeks in these mice, and resembled the degeneration seen early in the SCA-1 82Q B05 transgenic line. The only difference was the length of time required for pathology to appear. Thus, even wild-type, unexpanded human ataxin-1 produced a neurodegenerative phenotype when expressed at high levels.

### 6.3. EXAMPLE

### Ataxin-1 Causes Progressive Degeneration in Drosophila Neurons

An important feature of neural degeneration in SCA-1 and related diseases is that the phenotype worsens with the age of the patient (Zoghbi, and Orr, 2000, Annu. Rev. Neurosci. 23:217-247). This was also a feature of the SCA-1 transgenic mouse model system (Clark et al., 1997, J Neurosci 17:7385-95). Thus we investigated whether SCA-1 neurodegeneration in flies was also progressive.

The ap^{VNC}GAL4 driver was chosen because it drove expression to a small number of well-defined interneurons in the ventral nerve cord of the adult central nervous system (CNS). This driver was generated using a CNS-specific enhancer from the apterous (ap) gene (SEQ ID NO:1). Both the cell body and axonal projections of these interneurons can be easily visualized with the τ-GFP reporter gene. In the adult ventral nerve cord, there are two large interneurons per hemisegment (one dorsal and one ventral) that are strongly labeled by τ-GFP driven by the ap^{VNC}GAL4 enhancer. These interneurons extend their axons medially and anteriorly (FIGS. 3A and 3B). The integrity of these intemeurons was assessed at days 1, 25 and 45 of adult life in wild type and transgenic flies expressing ataxin-1 30Q or 82Q. As shown in FIG. 3C-D and Table 1, a progressive elimination of the cell bodies and axonal projections with age upon ataxin-1 82Q expression was observed. Similar but weaker phenotypes were observed with a high-expressing ataxin-1 30Q line (not shown). Expression of ataxin-1 in fly neurons, then, clearly duplicated the progressive degeneration observed in SCA-1 patients and transgenic mice.

TABLE 1
ataxinl-82Q causes progressive degeneration of interneurons
in the adult CNS

| Genotype | Days after eclosion | n | # interneurons |
|---|---|---|---|
| ap^{VNC}-GAL/UAS-LacZ(1) | 45 | 10 | 8.0 |
| ap^{VNC}-GAL/UAS-SCA-1- | 1 | 7 | 7.0 ± 1.15 |
| 82[M6](2) | 25 | 6 | 6.0 ± 0.9 |
| | 45 | 7 | 4.1 ± 1.6 |

Complete genotypes: (1) UAS: tauGFP/+; apterous^{VNC}-GAL4[UAS: LacZ; (2) UAS: τGFP/+; apterous^{VNC} GAL4fUAS: SCA-1-82[M6]. Only the interneurons of the thoracic segments 1 and 2 were analyzed (2 per hemisegment). In controls the intemeurons were scored by expression of GFP or LacZ. SCA-1-expressing neurons were scored by positive staining with anti-SCA-1 antibody.

### 6.4. EXAMPLE

### Ataxin-1 in Drosophila Forms Nuclear Inclusions that Accumulate Ubiquitin, the Proteasome and Molecular Chaperones

It was discovered that ataxin-1 30Q and ataxin-1 82Q accumulated in one or multiple NI unless expression levels were very low. By studying the NI at various time points after SCA-1 expression from a heat shock promoter, it was found that the nuclear inclusions were dynamic structures. Small 30Q and 82Q inclusions, which were visible shortly after expression, aggregated into bigger NI with time; this was particularly obvious with 82Q (not shown). Three factors were found to be important for nuclear inclusion formation: length of the polyglutamine domain, expression level, and length of time since onset of expression.

NI accumulated in a variety of cell types, including eye photoreceptor cells (see insets in FIG. 1H-1I), neurons of the CNS (FIG. 3C-3D), and cells of the mature salivary glands. These post-mitotic cells contained giant polytenic nuclei, so they were particularly useful for studying the morphology and aggregation dynamics of NI formed by ataxin-1 30Q and 82Q. In these giant nuclei, ataxin-1 30Q usually accumulated in a compact, oval aggregate, whereas ataxin-1 82Q accumulated in several irregularly-shaped aggregates (see FIG. 4).

We used molecular markers to study the inclusions formed by ataxin-1 in Drosophila, and compared them to the inclusions observed in SCA-1 patients and in the SCA-1 mouse model. The NI in both humans and mice accumulate the Hsp70 molecular chaperone, ubiquitin and components of the proteasome (Cummings et al., 1998, Nat Genet 19:148-54), presumably because the cell is attempting to refold and degrade mutant ataxin-1. FIG. 4D-L show that ataxin-1 NI in Drosophila are positive for the Hsp70 chaperone, ubiquitin, and the proteasome. Unlike Hsp70, Hsp90 did not accumulate in NI in SCA-1 patients or transgenic micel (Cummings et al., 1998, Nat Genet 19:148-54). We investigated Hsp83 (the Drosophila Hsp90 orthologue), and found that it does not accumulate in the NI of neurons or giant salivary gland nuclei (data not shown).

In summary, ataxin-1 NI in Drosophila were very similar to the inclusions observed in SCA-1 patients, transgenic mice and transfected cells, both with respect to the markers accumulated and to patterns of aggregation.

### 6.5. EXAMPLE

### Reducing the Activity of Genes Encoding Either Molecular Chaperones or a Component of the Proteasome Aggravates SCA-1 Neurodegeneration

Since ataxin-1 is degraded by the ubiquitin-proteasome pathway (Cummings et al., 1999, Neuron 24:879-92), we expected that a reduction of proteasome activity should aggravate the neurodegenerative phenotype. Similarly, it was predicted that decreased molecular chaperone activity would worsen the phenotype. An alternative possibility was that chaperones might themselves contribute to pathogenesis if they served to stabilize toxic folding intermediates (Krobitsch and Lindquist, 2000, Proc Natl Acad Sci USA 97:15 89-94). If this was the case, a moderate reduction of chaperone activity might actually improve the phenotype (Ferrigno and Silver, 2000, Neuron 26:9-12).

To test these hypotheses we took advantage of existing mutations in Drosophila genes encoding molecular chaperones or components of the proteasome. We obtained the following mutants: 1) Df (3R) karD1, a small deletion removing a cluster of hsp70 genes (see legend to FIG. 5). 2) hsc70-4¹⁹⁵, a point mutation in the ATP-binding domain of hsp70 cognate 4 protein. 3) a point mutation in Pros26, a gene encoding a multicatalytic endopeptidase which is a component of the 20S core proteasome. These mutants were crossed with flies expressing a SCA-1 82Q transgene producing an intermediate phenotype in the eye. As shown in FIG. 5, SCA-1 82Q[F7] transgenic flies heterozygous for any of these mutations showed a more severe eye phenotype than flies carrying only the SCA-1 82Q transgene. Control heterozygous flies carrying these mutations alone showed a wild-type eye phenotype (FIG. SF, and data not shown), demonstrating that partial reduction of the activities of Hsp70, Hsc70 or the proteasome aggravated the eye neurodegeneration phenotype.

### 6.6. EXAMPLE

### Genetic Screens for Modifiers of Ataxin-1-Induced NeurodeGeneration

The results described above with chaperones and proteasome mutants indicated that it was possible to carry out an F1 genetic screen to identify genes that alter ataxin-1-induced neurodegeneration when their activities were partially reduced.

To identify novel genes capable of modifying SCA 1-induced neurodegeneration, two genetic screens were carried out. First a collection of 1500 lethal P-element insertions was crossed with a UAS: SCA-11[82Q]F7; gmr-GAL4/CyO strain. In these flies a moderate level of ataxin-1 82Q accumulated in the retina, causing an intermediate eye phenotype at 25° C. A second genetic screen was performed to identify genes that, when overexpressed, suppressed or enhanced SCA 1-induced neurodegeneration. In this second instance, the UAS: SCA-1[82Q]F7; gmr-GAL4/CyO strain transgenic flies was crossed with a collection of 3000 EP insertions (Rorth et al., 1998, Development 125:1049-57). Putative modifiers detected in the initial screens were tested again to verify their phenotype and to investigate their specificity. Modifiers that altered the eye phenotype of SCA-1 transgenic but not control sibling flies were further studied.

The P-element F1 screen identified 27 modifier genes of the SCA-1 eye phenotype, 7 of which suppressed the SCA-1 phenotype and 20 of which enhanced the SCA-1 phenotype when their activity was reduced up to 50% by the P element insertion. The EP-element F1 screen produced a total of 33 modifiers of the SCA-1 phenotype, 10 of which suppressed the SCA-1 phenotype and 23 of which enhanced the SCA-1 phenotype. In principle, the eye phenotype modifications in the EP element screen may be caused by overexpression of a nearby transcription unit, but lack of function caused by insertional mutagenesis underlies some modifiers (see below, and Tables 2 and 3).

To identify the genes tagged by the P and EP insertions, genomic DNA sequences adjacent to the insertions were recovered by plasmid-rescue and inverse-PCR techniques. These sequences were then compared with the Drosophila genome databases. Some of the candidate genes affected by the P/EP insertions were not previously characterized; others were well known.

Some of the identified modifiers were genes in the protein folding/heat-shock response or ubiquitin-proteolytic pathways (Table 2 and FIG. 6).

TABLE 2
Modifiers in the protein folding heat-shock response,
and ubiquitin-proteolytic pathways.

| | | | | | | EP | |
|---|---|---|---|---|---|---|---|
| | | | | | | Orientat./ | |
| Line | Modification | Gene | Function | Accession# | Insertion | Overexp. | LOFA/M |
| P1666 | Enhances | Ubi63E | Ubiquitin | M22428 (cDNA) | −1913 | — | Yes (1)/ |
| | SCA-1 | | | AAA28997 (P) | | | Enhances |
| | | | | | | | SCA-1 |
| P1779 | Enhances | UbcD1 | Ubi | X62575 (cDNA) | −432 | — | Yes (2)/ |
| | SCA-1 | | conjugase | CAA44453 (P) | | | Enhances |
| | | | | | | | SCA-1 |
| EP674 | Enhances | UbcD1 | Ubi | X62575 (cDNA) | −484 | O/No | Yes (2)/ |
| | SCA-1 | | conjugase | CAA44453 (P) | | | Enhances |
| | | | | | | | SCA-1 |
| EP1303 | Enhances | dUbc-E2H | Ubi | AE003442 | −1439 | O/No | NA |
| | SCA-1 | | conjugase | (gDNA) | | | |
| | | | | SPTREMBL | | | |
| | | | | Q9W3K7 (P) | | | |
| P292 | Enhances | hsr-ω | Heat- | U18307 (cDNA) | −240 | — | Yes (3)/ |
| | SCA-1 | | shock | SPTREMBL | | | Enhances |
| | | | response | Q24022 (P) | | | SCA-1 |
| | | | factor | | | | |
| EP411 | Suppresses | DnaJ-1 64EF | chaperone | U34904 (cDNA) | −600 | S/Yes | NA |
| | SCA-1 | | | AAC23584 (P) | | | |

Insertion refers to insertion site relative to putative ATG at +1.
gDNA = genomic DNA sequence;
cDNA = cDNA or mRNA sequence;
P = protein sequence.
Orientation of P1 EP realtive to transcript unit,
S = same.
O = opposite.
LOFA: other loss of function alleles of the modifier gene.
M: modification of SCA-1 eye neurodegeneration caused by LOFA.
(1) Two EMS-induced alleles of P1666.
(2) UbcD1⁵¹⁷⁸².
(3) hsr-ω⁰⁵²⁴¹.

As described above, these pathways were known to be involved in polyglutamine-induced neural degeneration. In addition, novel modifiers were recovered, some of which identify pathways not previously known to be involved in polyglutamine-induced neurodegeneration (Table 3 and FIG. 7).

TABLE 3
Modifiers in novel pathways:

| | | | | | | EP | |
|---|---|---|---|---|---|---|---|
| | | | | | | Orientat./ | |
| Line | Modification | Gene | Function | Accession# | Insertion | Overexp. | LOFA/M |
| EP2231 | Suppresses | Gst-θ1 55F | Glutathione-S- | AF179869 (cDNA) | −78 | S/Yes | Yes (1)/ |
| | SCA-1 | | transferase | AAF64647 (P) | | | Enhances |
| | | | | | | | SCA-1 |
| EP2417 | Suppresses | nup44A | nucleoporin | AE002787 (gDNA) | −257 | S/Yes | Yes (2)/ |
| | SCA-1 | | | SPTREMBL | | | Enhances |
| | | | | Q9V348 (P) | | | SCA-1 |
| EP3623 | Suppresses | mub | RNA binding | X99340 (gDNA) | (A) | S/Yes | Yes (3)/ |
| | SCA-1 | | | CAA67719 (P) | | | Enhances |
| | | | | | | | SCA-1 |
| EP3461 | Enhances | pum | RNA binding | L07943 (cDNA) | (B) | S/Yes | NA |
| | SCA-1 | | | AAB59189 (P) | | | |
| EP3378 | Enhances | cpo | RNA binding | Z14311(cDNA) | −685 | S/Yes | Yes (4)/ |
| | SCA-1 | | | CAA78663 (P) | | | Enhances |
| | | | | | | | SCA-1 |
| EP3725 | Enhances | dYT521-B | RNA binding | AF145594 (cDNA) | −437 | S/Yes | NA |
| | SCA-1 | | | AAD38569 (P) | | | |
| EP866 | Enhances | Sin3A | Transcriptional | AJ007518 (cDNA) | +4601 | O/No | Yes (5)/ |
| | SCA-1 | | cofactor | CAA07550 (P) | | | Enhances |
| | | | | | | | SCA-1 |
| EP3672 | Enhances | Rpd3 | Transcriptional | AF086715 (cDNA) | −1363 | S/No | Yes (6)/ |
| | SCA-1 | | cofactor | AAC61494 (P) | | | Enhances |
| | | | | | | | SCA-1 |
| P1590 | Enhances | dCtBP | Transcriptional | AJ224690 (cDNA) | −7353 | — | Yes (7)/ |
| | SCA-1 | | cofactor | CAA12074 (P) | | | Enhances |
| | | | | | | | SCA-1 |
| EP2300 | Enhances | dSir2 | Transcriptional | AF068758 (cDNA) | −427 | S/Yes | NA |
| | SCA-1 | | cofactor | CAA79684 (P) | | | |
| P198 | Enhances | Pap/Trap | Transcriptional | AF226855 (cDNA) | +800 | — | Yes (8)/ |
| | SCA-1 | | cofactor | AAF36691 (P) | | | Enhances |
| | | | | | | | SCA-1 |
| EP3463 | Enhances | tara | Transcriptional | AF227213 (gDNA) | (C) | S/(D) | Yes (9)/ |
| | SCA-1 | | cofactor | AAF43019 (P) | −533 | | Enhances |
| | | | | | +16335 | | SCA-1 |

See Table 2 legend for symbols;
= means no modification of SCA-1 eye phenotype by loss of function allele (LOFA).
(A) inserted 22 base pairs into 5′ non-coding exon, separated from ATG by ˜28 kb intron.
(B) inserted in intron between exon 8 and exon 9.
(C) inserted in intron ˜16.3 kb downstream of first ATG, but only −553 with respect of second ATG.
(D) isoform 1A (starting at +16335 is disrupted, whereas isoform 1B starting at −553 is overexpressed.
(1) Imprecise excision of EP2231.
(2) mub⁰⁴⁰⁹³.
(3) pum¹³.
(4) cpo¹ and cpo^{L1}.
(5) Sin3A^{dQ4} and 5in3A⁰⁸²⁶⁹.
(6) Rpd3⁰⁴⁵⁵⁶.
(7) dCtBP^{87De-10}.
(8) pap⁵³ and pap^{EP1}.
(9) Five imprecise excisions of EP3463.

### 6.7. EXAMPLE

### Modifiers in the Protein Folding Heat-Shock Response and Ubiquitin Proteolytic Pathways

Four SCA-1 enhancers that identified three genes in the ubiquitin-proteolytic pathway were isolated by the SCA-1 modifier screens. P 1666 (FIG. 6B) was an insertion in a gene encoding ubiquitin (Ubi63E). Two EMS alleles of P 1666 were generated, and these mutations also behaved as enhancers of the eye phenotype (not shown). A mutation in a Ubiquitin C-terminal hydrolase, a protein involved in the recycling of Ubiquitin, was further analyzed. This mutant (Uch-L3^{2B8}) also enhanced the SCA-1 eye phenotype (not shown). P1779 (FIG. 6C) and EP674 (not shown) were mutations in UbeD1/effete, a gene that encoded a Ubiquitin conjugase homologous to human UbcE2D2 (93% identity) and yeast Ubc4/5 (81% identity) (Treier et al., 1992, Embo J 11:367-72). Another allele of UbcDl/effete was tested to verify the interaction (not shown). EP1303 (FIG. 6D) identified a different ubiquitin conjugase, previously uncharacterized in flies, that is most similar to human Ubc2EH (72% identity) and S. cerevisiae Ubc8 (57% identity).

Two additional modifiers identified genes in the protein folding/heat-shock response pathway. P292 is an enhancer (FIG. 6E) associated with a mutation in a poorly understood heat-shock response factor known as hsr-ω. This gene, which is required for viability and conserved between species (Lakhotia and Sharma, 1996; McKechnie et al., 1998, Proc NatI Acad Sci USA 95:2423-8), encodes nuclear and cytoplasmic RNAs but not a protein; it is expressed in unstressed cells, but is also rapidly induced by heat shock. Another existing allele of hsr-ω was tested, and was also shown to enhance SCA-1 neurodegeneration (not shown). EP411 (compare FIGS. 6H and 6I with 6F and 6G) is associated with overexpression of a Drosophila DNA J-1 gene (dDnaJ-1 64EF); this overexpression suppresses the SCA-1 phenotype. This gene encodes a protein homologous to the human chaperone HSP40/HDJ-1 (50% identity over 117 amino acids). Interestingly, an alteration of the NI was found in dDNAJ-1 64EF overexpressing flies. As shown in FIG. 6K, NI in these flies are more compact, and they occupy a smaller portion of the nucleus than the typical ataxin-1 82Q control NI (FIG. 6J). Overall they resemble the NI characteristic of ataxin-1 30Q (compare with FIG. 4B-C).

### 6.8. EXAMPLE

### Novel Genes and Pathways Involved in Neural Degeneration

Three modifiers that identify genes and pathways not previously known to be involved in neural degeneration were identified in the SCA-1 modifier screens.

EP2231 (FIGS. 7B and 7F), which suppresses the SCA-1 phenotype, caused the overproduction of a Glutathione-S-transferase gene (Gst55F) that is most similar to human GSTs of the theta class. GSTs are a group of enzymes that play important roles in cellular detoxification. They catalyze the conjugation of a variety of toxic compounds with reduced glutathione, which in turn facilitates their metabolism and excretion (Whalen and Boyer, 1998, Semin Liver Dis 18:345-58; Salinas et al., 1999, Curr Med Chem 6:279-309). The fly GST gene overexpressed in EP2231 is part of a cluster containing a total of ten GST genes in chromosomal position 5SF (unpublished). Conversely, imprecise excisions of EP2231 were generated that enhanced the SCA-1 phenotype (FIG. 7J). Two loss-of-function mutations (P1480 and P874) in Gst2 were additionally analyzed for their effect on the SCA-1 phenotype. Gst-2 is a different Gst gene mapping to chromosomal location 53F, and most similar to the human GST sigma class. These mutations also enhanced the SCA-1 eye phenotype (FIG. 7K shows P1480; P874 not shown).

EP2417 (FIGS. 7C and 7G), which suppresses the SCA-1 phenotype, is associated with overexpression of an uncharacterized Drosophila gene that the inventors have named nucleoporin-44A (nup-44A). nup-44A encodes a protein homologous to the S. cerevisiae nuclear pore protein SEHi (34% identity over 185 amino acids).

Among the other novel modifiers of SCA-1 neurodegeneration, four genes were found to contain proteins with RNA-binding domains:

EP3623 (FIGS. 7D and 7H), which suppresses the SCA-1 phenotype, overexpressed mushroom-body expressed (mub), which encodes a protein similar to vertebrate RNA-binding KH-domain proteins. It is thought to bind and stabilize specific mRNAs (Grams and Korge, 1998,Gene 215:191-201). A mub loss of function allele does not modify the eye phenotype (not shown).

EP3461 (FIG. 7L), which enhances the SCA-1 phenotype, overexpresses exons 9-13 of the pumilio (pum) transcription unit including thepum RNA-binding domains. Using pum antibodies, the overproduction of the Pumilio protein was confirmed. pum regulates translation of specific mRNAs by recruiting cofactors to its RNA binding sites (Sonoda and Wharton, 1999, Genes Dev 13:2704-12).

EP 3378 (FIG. 7M), which enhances the SCA-1 phenotype, is inserted in couch potato (cpo). This gene, expressed in CNS and PNS cells, encodes a nuclear RNA-binding protein (Bellen et al., 1992, Gen. Dev. 6:2125-2136). Using Cpo-specific antibodies, cpo overexpression in EP3378 (not shown) was verified. Loss of function alleles of cpo, do not modify the ataxin-1 82Q eye phenotype (not shown).

EP3725 (FIG. 7N), which enhances the SCA-1 phenotype, overexpresses an uncharacterized Drosophila gene encoding a protein homologous to the rat splicing factor YT52 1-B (37% identity over 287 amino acids).

Analysis of six additional EP elements which enhance the SCA-1 phenotype identified proteins that function as transcriptional regulators:

EP866 (FIG. 7O) is a loss of function mutation in Sin3A, the fly homolog of the mouse Sin3A and yeast Sin3p corepressors (Pennetta and Pauli, 1998, Dev Genes Evol 208:53 1-6). Other Sin3A alleles also enhanced the SCA 1 eye phenotype (not shown).

EP3672 (FIG. 7L) is a loss of function mutation in the Rpd3 gene that encodes a histone deacetylase (De Rubertis et al., 1996, Nature 384:589-591). A different Rpd3 allele also enhances the SCA-1 phenotype (not shown). Both Sin3A and Rpd3 are part of a large protein complex required for transcriptional repression (Kasten et al., 1997, Mol Cell Biol 17:4852-8).

EP 1590 (FIG. 7Q) is a mutation in the dCtBP corepressor (Nibu et al., 1998, Science 280:101-4). Another dCtBP allele enhances the SCA-1 eye phenotype (not shown).

EP2300 (FIG. 7R) overexpresses the fly homolog of the yeast protein Sir2, a chromatin remodeling factor required for silencing (Laurenson and Rine, 1992, Microbiol Rev 56:543-60).

EP198 (FIG. 7S) is an insertion in the gene poils auxpattes (pap). Mutations in pap were independently isolated as genetic interactors with the proboscipedia Hox transcription factor (D. L. Cribbs, personal communication). pap is the fly homolog of human Trap240, a component of the TRAP/SMCC cofactor protein complex involved in transcriptional regulation (Ito et al., 1999, Mol Cell 3:361-70). TRAP/SMCC is related to the yeast Mediator complex that interacts with RNA polymerase II and has co-activator and corepressor functions, reviewed in Hampsey, 1998, Microbiol Mol Biol Rev 62:465-503. The interaction was verified with other pap alleles (not shown).

EP3463 (FIG. 7T) is an insertion within the taranis (tara) transcription unit. tara is a member of the trithorax group of transcription factors (D. L. Cribbs, personal communication). Five imprecise excisions of EP3463 were generated, and they caused the same severe enhancement of the SCA-1 eye phenotype (not shown).

### 6.9. EXAMPLE

### Additional Genes and Pathways Involved in Neural Degeneration

Additional modifiers of the Drosophila rough eye SCA-1 phenotype are listed in table 4, infra. The modifiers listed in table 4 can be used in accordance with the methods of the invention. For each P or EP element listed in table 4 that modifies the SCA-1 phenotype, the identity of the genes neighboring the sites of the P or EP element insertion are listed.

TABLE 4
Additional SCA-1 modifiers:

| | Effect on | | | | Insert/orient | |
|---|---|---|---|---|---|---|
| Line | SCA1 | Gene | Accession No. | Function | (ATG = +1) | |
| EP(2)0340 | suppresses | CG6785 | AE003634 (gDNA)* | Unknown | −1828 | O |
| | | | AAF53150 (P) | | | |
| EP(X)0355 | enhances | Dsp1 | U13881 (cDNA) | Transcription | −2802 | S |
| | | | JC6179 (P) | factor | | |
| EP(2)0467 | suppresses | HmgD | AE003455 (gDNA)* | HMG box | −2170 | S |
| | | | AAF46759 (P) | transcriptional | | |
| | | | | regulator | | |
| EP(2)0538 | enhances | CG10934 | AE003802 (gDNA)* | Unknown | −69 | S |
| | | | AAF57809 (P) | | | |
| EP(3)0559 | enhances | CG3445 | AE003552 (gDNA)* | zinc finger | −569 | S |
| | | | CG3445 (P) | | | |
| EP(3)0565 | suppresses | CG6783 | AE003692 (gDNA)* | fatty acid binding | −240 | S |
| | | | AAF54655 (P) | | | |
| EP(3)0635 | enhances | Xnp | AF217802 (gDNA)* | DNA helicase | −471 | S |
| | | | AAG40586 (P) | | | |
| EP(3)0678 | enhances | CG1910 | AE003779 (gDNA)* | Unknown | −318 | S |
| | | | AAF57190 (P) | | | |
| EP(2)0787 | enhances | CG5261 | AE003617 (gDNA)* | dH acetyl | −3285 | S |
| EP(2)1221 | | | AAF52514 (P) | transferase | −3435 | S |
| | | | AAF52515 (P) | | | |
| EP(3)0872 | suppresses | CG4834 | AE003753 (gDNA)* | Unknown | −864 | S |
| | | | AAF56498 (P) | | | |
| EP(2)1127 | suppresses | CG18445 | AE003831 (gDNA)* | Unknown | −223 | S |
| | | | AAF58858 (P) | | | |
| EP(2)1211 | enhances | Lilliputian/ | AE003581 (gDNA)* | Transcriptional | −57434 | S |
| | | CG8817 | AAF51180 (P) | regulator | | |
| EP(X)1300 | enhances | CG8062 | AE003511 (gDNA)* | Monocarboxylic | −8459 | S |
| | | | AAF48949 (P) | acid transporter | | |
| EP(X)1331 | enhances | Act5C/ | AE003435 (gDNA)* | Structural protein | −730 | O |
| | | CG4027 | AAF46098 (P) | | | |
| EP(X)1357 | enhances | CG8240 | AE003506 (gDNA)* | Rho GTPase | −1286 | S |
| | | | AAF48749 (P) | activator | | |
| EP(X)1438 | suppresses | CG14438 | AE003438 (gDNA)* | Zinc finger | −275 | S |
| | | | AAF46197 (P) | | | |
| EP(X)1617 | enhances | CG9650 | AE003440 (gDNA)* | Zinc finger | −36244 | S |
| | | | AAF46246 (P) | | | |
| EP(2)2004 | enhances | CG7233 | AE003619 (gDNA)* | Transcriptional | −941 | S |
| | | | AAF52581 (P) | regulator | | |
| EP(2)2038 | enhances | pipsqueak | U48358 (cDNA) | Transcription | −18450 | S |
| | | | U48402 (cDNA) | factor | | |
| EP(2)2039 | enhances | elbow B | AE003409 (gDNA)* | Transcription | −16735 | S |
| | | | Q9NKC3 (P) | factor | | |
| EP(2)2058 | suppresses | CG10882 | AE003583 (gDNA)* | Serine-type | −251 | O |
| | | | AAF51283 (P) | endopeptidase | | |
| EP(2)2227 | enhances | CG14757 | AE003838 (gDNA)* | Unknown | −1185 | O |
| | | | AAF59116 (P) | | | |
| EP(2)2197 | enhances | CG8204 | AE003810 (gDNA)* | Unknown | −40029 | S |
| | | | AAF58118 (P) | | | |
| EP(2)2425 | enhances | CG12846 | AE003842 (gDNA)* | Tetraspanin | −28378 | S |
| | | | AAF59312 (P) | | | |
| EP(3)3091 | suppresses | Pk61C | AE003467 (gDNA)* | Protein kinase | +5415 | O |
| | | | AAF47327-32 (cDNA) | | | |
| EP(3)3118 | enhances | Rac2 | L38310 (cDNA) | Rho small | −793 | S |
| | | | AAA67041 (P) | GTPase | | |
| EP(3)3139 | suppresses | CG14959 | AE003477 (gDNA)* | Chitin-binding | −16380 | S |
| EP(3)3041 | | | AAF47749 (P) | peritrophin A and | | |
| | | | | type 2 domains | | |
| EP(3)3145 | enhances | CG5166 | AE003708 (gDNA)* | ataxin-2 like | −3121 | S |
| | | | AAF55196 (P) | | | |
| EP(3)3183 | enhances | CG14363 | AE003701 (gDNA)* | | −22408 | S |
| | | | AAF54993 (P) | | | |
| EP(3)3659 | enhances | boule | S68988 (P) | RNA binding | −12085 | S |
| | | | U51858 (cDNA) | | | |
| EP(3)3673 | enhances | CG12084 | AE003471 (gDNA)* | Armadillo repeat | −1751 | S |
| | | | AAF47500 (P) | | | |
| P(3)276 | enhances | CG7518 | AE003698 (gDNA)* | Unknown | −128 | |
| | | | AAF54888 (P) | | | |
| P(3)308 | enhances | vibrator/ | AE003725 (gDNA)* | PITP transfer | +574 | |
| | | CG5269 | AAF55650 (P) | | | |
| P(2)662 | suppresses | CG9246 | AE003669 (gDNA)* | Unknown | −16 | |
| | | | AAF53971 (P) | | | |
| P(2)691 | suppresses | CG11171 | AE003791 (gDNA)* | WD repeat | −223 | |
| | | | AAF57440 (P) | | | |
| P(2)1030 | suppresses | pKa-C1 | AE003625 (gDNA)* | protein ser/thr | −1018 | |
| | | | AAF52797 (P) | kinase | | |
| P(2)1053 | enhances | KEK1 | U42767 (cDNA) | tyrosine | −1222 | |
| | | | AAF53225 (P) | phosphatase | | |
| P(2)1066 | suppresses | CG6301 | AE003805 (gDNA)* | Unknown | | |
| | | | AAF57947 (P) | | | |
| P(2)1214 | enhances | lesswright | AB017607 (cDNA) | Ubc9, ubiquitin | | |
| | | | BAA34575 (P) | conjugase | | |
| P(2)1295 | enhances | spen | AE003590 (gDNA)* | RNA binding | −1019 | |
| | | | AF51534 (P) | | | |
| | | | AF51535 (P) | | | |
| P(2)1335 | enhances | mastermind | P21519 (P) | | | |
| | | | X54251 (cDNA) | | | |
| P(3)1536 | enhances | CG11278/ | AE003539 (gDNA)* | t-SNARE | −432 | |
| | | Syx13 | AAF49845 (P) | | | |
| P(3)1549 | enhances | CG6767 | AE003551 (gDNA)* | PRPP synthetase | +1589 | |
| | | | AAF50223 (P) | | | |
| | | | AAF50224 (P) | | | |
| P(3)1607 | enhances | CG5891 | AE003528 (gDNA)* | Unknown | +15774 | |
| | | | AAF49547 (P) | | | |
| P()1619 | enhances | CG10733 | AE003564 (gDNA)* | intracellular | −104 | |
| | | | AAF50698 (P) | trafficking | | |
| | | | | emp24/gp25L/p24 | | |
| P(3)1678 | enhances | jumu | AB028890 (cDNA) | transcription | +5972 | |
| | | | | factor | | |
| P(3)1737 | enhances | pebble | AF136492 (cDNA) | Guanidyl | −450 | |
| | | | AAD52845 (P) | nucleotide | | |
| | | | | exchange factor | | |
| P(3)1796 | enhances | shank | | Unknown | | |
| P(3)1797 | enhances | hsp83 | AE003477 (gDNA)* | Chaperone | | |
| | | | AAF47734 (P) | | | |
| P(3)2112 | enhances | tacc | AE003605 (gDNA)* | Microtubule | +9226 | |
| | | | AAF52099 (P) | stabilization | | |
| P(2)2175 | suppresses | guftagu | Q9V475 (P) | Cullin E3-ligase | −404 | |
| P(3)2160 | enhances | CG9988 | AE003766 (gDNA)* | Unknown | −2173 | |
| | | | AAF56804 (P) | | | |
| P(2)2341 | suppresses | ariadne-2 | AJ010169 (cDNA) | E3-ligase | −558 | |
| | | | CAA09030 (P) | | | |
| P(2)2346 | suppresses | Gbp | AE003799 (gDNA)* | GTP binding | −412 | |
| | | | AAF57684 (P) | protein | | |
| P(2)1180 | Suppresses | CG25C | COLLAGE IV** | U65431 | −343 | |
| | SCA-1 | VICKING | COLLAGE IV | M96575 | −2745 | |
| P(3)1642 | Suppresses | HID | CELL DEATH** | U31226 | +315 | |
| | SCA-1 | | | | | |
| P(3)0344 | Enhances | CG6338 | TRANSCRIPTION | AEOO3758 | −3079 | |
| | SCA-1 | | FACTOR | | | |
| P(3)1498 | Enhances | SNAP25 | SYNAPTIC PROTEIN | AE003379 | −3254 | |
| | SCA-1 | | | | | |
| P(3)1622 | Enhances | CG6983 | UNKNOWN | AE003555 | −1059 | |
| | SCA-1 | | FUNCTION** | | | |
| | | GRUNGE | DNA PACKING | AE003555 | −11603 | |

See Table 2 legend for symbols.
*indicates that the accession no. refers to a genome project “scaffold” with a number of genes; however, this information is annotated to identify which residues code for the SCA-1 modifier listed in the table above.
**indicates the preferred embodiment.

### 6.10. EXAMPLES

### Discussion

The inventors have generated a Drosophila model for SCA 1-induced neurodegeneration that replicates the main features of pathogenesis observed in human polyglutamine diseases. Expression of an expanded human SCA-1 transgene encoding ataxin-1 82Q led to degeneration of Drosophila neurons and NI formation. As in SCA-1 patients, SCA-1 neurodegeneration in flies was progressive, as shown by monitoring the integrity of the cell bodies and projections of adult intemeurons at different ages (FIG. 3 and Table 1).

Among the transgenic lines producing ataxin-1 82Q, strong, intermediate, and weak phenotypes were detected. The phenotypes correlated directly with expression levels. The ataxin-1 30Q lines produced weaker phenotypes than the 82Q lines, even though expression levels were similar. That overexpression of ataxin-1 30Q was able to elicit mild phenotypes was somewhat unexpected, because neural degeneration was not previously reported with this wild-type human isoform. Mice carrying two copies of the SCA-1 30Q transgene also showed neurodegeneration. Only higher levels of expression and prolonged exposure were required for the wild-type protein to exert toxic effects.

It thus appears that the gain of ataxin-1 toxicity normally associated with polyglutamine expansion can also result from excess wild-type protein. In this context, it was recently reported that overexpression of wild-type ct-synuclein in transgenic mice led to the formation of ubiquitin-positive inclusions and neural degeneration that resembled the Parkinsonian pathogenesis caused by mutant α-synuclein (Masliah et al., 2000, Science 287:1265-9).

Ataxin-1 accumulates in nuclear inclusions in transgenic flies. These aggregates are very similar to the aggregates observed in SCA-1 patients: they alter the subcellular localization of ubiquitin, the proteasome, and Hsp70, but not Hsp83 (Cummings et al., 1998, Nat Genet 19:148-54) (FIG. 4). As the inventors have shown here, overproduction of the Hsp70 and Hsp40 chaperones was able to suppress polyglutamine toxicity (see also, Warrick et al., 1999, Nat Genet 23:425-8; Kazemi-Esfarjani and Benzer, 2000, Science 287:1837-40). The inventors have also shown that Hsp40 modifies the NI, making them more defined and compact (FIG. 6K). These observations argue that high quantities of chaperones are protective. Although it has been suggested that a moderate decrease in chaperone activity might protect against disease progression (Ferrigno and Silver, 2000, Neuron 26:9-12), the inventors have discovered that reducing Hsp 70 activity aggravates the SCA-1 phenotype.

Using genetic screens to identify modifiers of polyglutamine-induced neurodegeneration, the inventors recovered suppressors and enhancers that modify the SCA-1 phenotype by partial loss of function or by gene overexpression. Some of these modifiers were involved in protein folding or proteolysis. One suppressor and several enhancers that belonged to this class. The suppressor is associated with overexpression of dDNAJ-1 64EF, the same gene identified in a screen for suppression of polyglutamine toxicity (Kazemi-Esfarjani and Benzer, 2000, Science 287:1837-40). The enhancers were loss of function alleles in the structural gene encoding ubiquitin, two ubiquitin conjugases (UbcDl and dUbc-E2H) and hsr-ω. The latter is a heat-shock response factor encoding a nuclear RNA. The finding that reducing the activities of these genes aggravates SCA-1 neurodegeneration demonstrates that these components of the protein folding/proteolytic machinery were limiting quantities in SCA 1-compromised cells, and further supported the hypothesis that polyglutamine diseases are, at least in part, diseases of protein clearance.

Perhaps the most interesting modifiers are those genes involved in molecular pathways not previously known to play a role in neurodegeneration. Some of these genes identify novel pathways through which misfolded ataxin-1 mediates its toxicity, and suggest the nature of other pathogenic mechanisms besides impaired protein clearance. One of the suppressors in this class is associated with overexpression of a Glutathione S-transferase gene in 55F. GSTs are enzymes that play a major role in cellular detoxification, and use reduced glutathione in conjugation and reduction reactions with a variety of toxins including products of chemical and oxidative stress (Whalen and Boyer, 1998, Semin Liver Dis 18:345-58; Salinas et al., 1999, Curr Med Chem 6:279-309). Mutations in a different Gst gene (Gst2) also enhance the SCA-1 phenotype. Thus, these findings demonstrate another pathway used by cells to counteract the toxic effects of ataxin-1 besides the protein-folding pathway. A second suppressor is associated with overexpression of a protein homologous to a component of the yeast nuclear pore. The nuclear pore complex is composed of many proteins (Bodoor et al., 1999, Biochem Cell Biol 77:32 1-9); overexpression of one component may thus impair nuclear pore complex formation and impede ataxin-1 import into the nucleus. This observation provides additional evidence for the hypothesis that ataxin-1 and other polyglutamine proteins exert their toxic effects in the nucleus.

Five RNA-binding proteins modify SCA-1 neurodegeneration—four enhance the phenotype, and one suppresses it. In addition, as mentioned above, the heat-shock factor hsr-ω encodes a nuclear RNA. These findings suggest that alteration of RNA processing is relevant to SCA-1 pathogenesis. Consistent with this is the observation that ataxin-1 binds RNA in vitro, suggesting that ataxin-1 is an RNA binding protein itself (Yue and Orr, unpublished data). That one of the modifiers in this class is a suppressor suggests that it may be possible to slow SCA-1 neurodegeneration by altering the activities of specific molecules involved in RNA processing. It is interesting to note that several diseases (Fragile X, Friedreich ataxia, myotonic dystrophy, and SCA-8) are caused by expansion of non-coding trinucleotide repeats. Thus, alteration of RNA processing or transport may be a recurring theme in trinucleotide repeat disorders.

A second group of enhancers identify proteins that function as cofactors in transcriptional regulation. This finding suggests possible abnormal interactions between ataxin-1 and the transcriptional machinery as an additional mechanism of pathogenesis. In this context, Lin and co-workers recently showed that alterations in gene expression occur very early during SCA-1 pathogenesis (Lin et al., 2000, Nat Neurosci 3:157-63). There are several means by which ataxin-1 might interfere with transcription which include: (1) perturbation of the proteolytic machinery could alter levels of important transcription factors whose concentrations are regulated by proteolysis; (2) mutant ataxin-1 may interfere with nuclear domains important for transcriptional regulation (Skinner et al., 1997, Nature 389:97 1-4); and (3) ataxin-1 may directly interact with certain components of the transcriptional machinery. Relatively short polyglutamine tracts are found in many transcription factors; thus, SCA-1 and other polyglutamine disease proteins may interfere with specific transcriptional regulators (see Waragai et al., 1999, Hum Mol Genet 8:977-87). The third possibility is supported by the finding that all modifiers in this class are transcriptional cofactors, but not other components of the transcriptional machinery. Also supporting this model is the recent observation that the N-terminus of huntingtin interacts with the nuclear receptor corepressor (N-CoR) in the yeast two-hybrid system (Boutell et al., 1999, Hum Mol Genet 8:1647-55).

The present invention is not to be limited in scope by the specific embodiments described herein. Indeed, various modifications of the invention in addition to those described herein will become apparent to those skilled in the art from the foregoing description and accompanying drawings. Such modifications are intended to fall within the scope of the appended claims.

Various references are cited herein above, including patent applications, patents, nucleic acid and protein accession numbers, and publications, the disclosures of which are hereby incorporated by reference in their entireties.

What is claimed is:

1. A transgenic Drosophila whose somatic and germ cells comprise a transgene operatively linked to a promoter, wherein the transgene encodes normal ataxin-1, wherein the expression of said transgene in the nervous system results in said Drosophila having a predisposition to progressive neural degeneration.

2. The transgenic Drosophila according to claim 1, wherein the transgene encodes ataxin-1 comprising a polyglutamine repeat having 6-19 glutamine residues.

3. The transgenic Drosophila according to claim 1, wherein the transgene encodes ataxin-1 comprising a polyglutamine repeat having 20-40 glutamine residues and 1-4 histidine residues.

4. A transgenic Drosophila whose somatic and germ cells comprise a transgene operatively linked to a promoter, wherein the transgene encodes ataxin-1 with expanded polyglutamine repeats, wherein the expression of said transgene in the nervous system results in progressive neural degeneration.

5. The transgenic Drosophila according to claim 4, wherein the transgene encodes ataxin-1 comprising a polyglutamine repeat having 39-82 glutamine residues.

6. The transgenic Drosophila according to claim 4, wherein the transgene is ataxin-1 82Q.

7. The transgenic Drosophila according to claim 1 or 4, wherein the transgene is operatively linked to a heterologous promoter.

8. The transgenic Drosophila according to claim 7, wherein the transgene is temporally regulated by the heterologous promoter.

9. The transgenic Drosophila according to claim 7, wherein the transgene is spatially regulated by the heterologous promoter.

10. The transgenic Drosophila according to claim 7, wherein the heterologous promoter is a heat shock promoter.

11. The transgenic Drosophila according to claim 10, wherein the heat shock promoter is derived from the hsp70 or hsp83 gene.

12. The transgenic Drosophila according to claim 7, wherein the transgene is operatively linked to a Gal4 Upstream Activating Sequence (“UAS”).

13. The transgenic Drosophila according to claim 8, further comprising a GAL4 gene.

14. The transgenic Drosophila according to claim 13, wherein the GAL4 gene is linked to a tissue specific promoter.

15. The transgenic Drosophila according to claim 14, wherein the tissue specific promoter is derived from the sevenless, eyeless, or glass genes.

16. The transgenic Drosophila according to claim 14, wherein the tissue specific promoter is derived from the dpp, vestigal, or apterous genes.

17. The transgenic Drosophila according to claim 14, wherein the tissue specific promoter is derived from the elav, Appl, or nirvana genes.

18. The transgenic Drosophila according to claim 7, wherein the heterologous promoter comprises a tetracycline-controlled transcriptional activator (tTA) responsive regulatory element.

19. The transgenic Drosophila according to claim 18, further comprising a tTA gene.

20. The transgenic Drosophila according to claim 19, wherein the tTA gene is operatively linked to a tissue specific promoter.

21. A method of screening for a molecule having activity against a neurodegenerative disorder, comprising:
(a) contacting a first transgenic Drosophila which expresses ataxin-1 with expanded polyglutamine repeats in its central nervous system with said molecule; and
(b) determining whether progressive neuronal degeneration in said transgenic Drosophila is less severe than progressive neuronal degeneration of a second Drosophila which expresses the ataxin-1 with expanded polyglutamine repeats in its central nervous system but wherein said second Drosophila was not contacted with said molecule;
wherein a reduction in the progressive neuronal degeneration of the first Drosophila relative to a the second Drosophila is indicative that the molecule has activity against a neurodegenerative disorder.

22. The method of claim 21, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

23. The method of claim 22, wherein the neurodegenerative disorder is a polyglutamine disease.

24. The method of claim 23, wherein the polyglutamine disease is spinocerebellar ataxia (SCA)-1, SCA-2, SCA-6, SCA-7, Machado-Joseph disease (MJD), Huntington Disease (HD), spinobulbar muscular atrophy (SBMA), or dentatorubropallidolusyan atrophy (DRPLA).

25. The method of claim 24, wherein the polyglutamine disease is SCA-1.

26. The method of claim 21, wherein the ataxin-1 comprises a polyglutamine repeat having 39-82 glutamine residues.

27. The method of claim 21, wherein the ataxin-1 with expanded polyglutamine repeats is ataxin-1 82Q.

28. The method of claim 21, wherein the first transgenic Drosophila is contacted with the molecule during the larval stages of development.

29. The method of claim 21, wherein the first transgenic Drosophila is contacted with the molecule during adulthood.

30. The method of claim 21, wherein the determination of neural degeneration takes place during the larval stages of development.

31. The method of claim 21, wherein the determination of neural degeneration takes place during adulthood.

32. The method of claim 21, wherein the determination of neural degeneration is determined by examining the ventral nerve cord of the central nervous system.

33. The method of claim 21, wherein the determination of neural degeneration is determined by examining nuclear inclusion formation.

34. The method of claim 21, wherein the expression of ataxin-1 is under the control of a Gal4 UAS element.

35. The method of claim 21, wherein the first and second Drosophila further comprise a GAL4 gene operatively linked to a tissue specific promoter.

36. The method of claim 35, wherein the tissue specific promoter is derived from the sevenless, eyeless, or glass genes.

37. The method of claim 35, wherein the tissue specific promoter is derived from the dpp, vestigal, or apterous genes.

38. A method of screening for a molecule having activity against a neurodegenerative disorder, comprising:
(a) contacting a first transgenic Drosophila larva which expresses ataxin-1 with expanded polyglutamine repeats in its eye imaginal disc with said molecule, which expression results in a rough eye phenotype; and
(b) determining whether the rough eye phenotype in a first adult Drosophila resulting from said first larva is less severe than the rough eye phenotype of a second adult Drosophila resulting from a second larva which expresses the ataxin-1 with expanded polyglutamine repeats in its eye imaginal disc but wherein said second larva was not contacted with said molecule;
wherein a reduction in the rough eye phenotype of the first adult Drosophila relative to a the second adult Drosophila is indicative that the molecule has activity against a neurodegenerative disorder.

39. The method of claim 38, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

40. The method of claim 39, wherein the neurodegenerative disorder is a polyglutamine disease.

41. The method of claim 40, wherein the polyglutamine disease is SCA-1, SCA-2, SCA-6, SCA-7, MJD, HD, SBMA, or DRPLA.

42. The method of claim 41, wherein the polyglutamine disease is SCA-1.

43. The method of claim 38, wherein the ataxin-1 comprises a polyglutamine repeat having 39-82 glutamine residues.

44. The method of claim 38, wherein the ataxin-1 with expanded polyglutamine repeats is ataxin-1 82Q.

45. The method of claim 38, wherein the expression of ataxin-1 is under the control of a Gal4 UAS element.

46. The method of claim 45, wherein the first and second Drosophila contain a GAL4 gene operatively linked to an eye specific promoter.

47. The method of claim 46, wherein the eye specific promoter is derived from the sevenless, eyeless, or glass genes.

48. A method of screening for a molecule having activity against a vertebrate disease, comprising:
(a) contacting a first transgenic Drosophila larva which expresses a vertebrate disease gene associated with said vertebrate disease in its central nervous system with said molecule, said expression of said vertebrate disease gene resulting in a behavioral disorder; and
(b) determining whether the behavioral disorder in a first adult Drosophila resulting from said first larva is less severe than the behavioral disorder of a second adult Drosophila resulting from a second larva which expresses said vertebrate disease gene in its central nervous system but wherein said second larva was not contacted with said molecule;
wherein a reduction in severity of the behavioral disorder of the first adult Drosophila relative to a the second adult Drosophila is indicative that the molecule has activity against the vertebrate disease.

49. The method of claim 48, wherein the expression of the vertebrate disease gene is under the control of a Gal4 UAS element.

50. The method of claim 49, wherein the first and second Drosophila further comprise a GAL4 gene operatively linked to a tissue specific promoter.

51. The method of claim 50, wherein the tissue specific promoter is derived from the elav, Appl, or nirvana genes.

52. The method of claim 48, wherein the behavioral disorder is a motor deficit.

53. The method of claim 48, wherein the vertebrate disease is a mammalian disease.

54. The method of claim 53, wherein the mammalian disease is a human disease.

55. The method of claim 48, wherein the vertebrate disease is a neurodegenerative disorder.

56. The method of claim 55, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

57. The method of claim 56, wherein the neurodegenerative disorder is a polyglutamine disease.

58. The method of claim 57, wherein the polyglutamine disease is SCA-1, SCA-2, SCA-6, SCA-7, MJD, HD, SBMA, or DRPLA.

59. The method of claim 58, wherein the polyglutamine disease is SCA-1.

60. The method of claim 48, wherein the vertebrate disease gene encodes ataxin-1 with expanded polyglutamine repeats.

61. The method of claim 48, wherein the vertebrate disease gene encodes tau, synuclein, prion protein, huntingtin, or ataxin-3.

62. The method of claim 48, wherein the vertebrate disease is a proliferative disorder.

63. The method of claim 62, wherein the proliferative disorder is cancer.

64. The method of claim 63, wherein the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenström's macroglobulinemia, or heavy chain disease.

65. The method of claim 48, wherein the vertebrate disease is skeletal muscle disorder.

66. The method of claim 65, wherein the skeletal muscle disorder is a muscular dystrophy, a motor neuron disease, or a myopathy.

67. A method of identifying a modifier gene of SCA-1, comprising:
(a) generating a cross between a transgenic Drosophila whose somatic and germ cells comprise a transgene operatively linked to a promoter, wherein the transgene encodes ataxin-1 with expanded polyglutamine repeats, wherein the expression of said transgene in the nervous system results in progressive neural degeneration; and a second Drosophila suspected of having one or more mutations in its germ cells, to produce progeny;
(b) determining whether the progeny of said cross have a modified phenotype associated with the ataxin-1 transgene, wherein a modification of the phenotype associated with the ataxin-1 transgene is indicative that the second Drosophila has a mutation in a modifier gene of SCA-1; and
(c) identifying the gene responsible for the modified phenotype associated with associated with the ataxin-1 transgene; wherein the gene identified in step (c) is a modifier gene of SCA-1.

68. The method of claim 67, wherein the ataxin-1 transgene encodes an ataxin-1 polypeptide comprising a polyglutamine repeat with 39-82 glutamine residues.

69. The method of claim 67, wherein the transgene is ataxin-1 82Q.

70. The method of claim 67, further comprising:
(d) identifying a mammalian homolog of said modifier gene of SCA-1.

71. The method of claim 67, wherein said mutation in said second Drosophila is caused by an EP-element.

72. The method of claim 71, wherein the EP-element harbors an upstream activating sequence.

73. The method of claim 67, wherein said modification of the phenotype associated with the ataxin-1 transgene is an enhancement of the phenotype, said mutation esponsible for the enhancement of the phenotype is a loss of function mutation, and said modifier gene of SCA-1 is an enhancer gene of SCA-1.

74. The method of claim 67, wherein said modification of the phenotype associated with the ataxin-1 transgene is a suppression of the phenotype, said mutation responsible for the for the suppression of the phenotype is a gain of function mutation, and said modifier gene of SCA-1 is an enhancer gene of SCA-1.

75. The method of claim 67, wherein said modification of the phenotype associated with the ataxin-1 transgene is a suppression of the phenotype, said mutation responsible for the for the suppression of the phenotype is a loss of function mutation, and said modifier gene of SCA-1 is a suppressor gene of SCA-1.

76. The method of claim 67, wherein said modification of the phenotype associated with the ataxin-1 transgene is an enhancement of the phenotype, said mutation responsible for the for the enhancement of the phenotype is a gain of function mutation, and said modifier gene of SCA-1 is a suppressor gene of SCA-1.

77. A method of identifying a modifier gene of SCA-1, comprising:
(a) crossing a transgenic Drosophila whose somatic and germ cells comprise a transgene operatively linked to a promoter, wherein the transgene encodes ataxin-1 with expanded polyglutamine repeats, wherein the expression of said transgene in the nervous system results in progressive neural degeneration, with a mutagenized Drosophila to produce progeny;
(b) determining whether the progeny of the cross of step (a) Drosophila have a modified phenotype associated with the ataxin-1 transgene, wherein a modification of the phenotype associated with the ataxin-1 transgene is indicative that the mutagenized Drosophila has a mutation in a modifier gene of SCA-1; and
(c) identifying the gene responsible for the modified phenotype associated with associated with the ataxin-1 transgene; wherein the gene identified in step (c) is a modifier gene of SCA-1.

78. The method of claim 77, wherein the ataxin-1 transgene encodes an ataxin-1 polypeptide comprising a polyglutamine repeat with 39-82 glutamine residues.

79. The method of claim 77, wherein the ataxin-1 transgene is ataxin-1 82Q.

80. The method of claim 77, further comprising:
(d) identifying a mammalian homolog of said modifier gene of SCA-1.

81. The method of claim 77, wherein said modification of the phenotype associated with the ataxin-1 transgene is an enhancement of the phenotype, said mutation responsible for the for the enhancement of the phenotype is a loss of function mutation, and said modifier gene of SCA-1 is an enhancer gene of SCA-1.

82. The method of claim 77, wherein said modification of the phenotype associated with the ataxin-1 transgene is a suppression of the phenotype, said mutation responsible for the suppression of the phenotype is a gain of function mutation, and said modifier gene of SCA-1 is an enhancer gene of SCA

83. The method of claim 77, wherein said modification of the phenotype associated with the ataxin-1 transgene is a suppression of the phenotype, said mutation responsible for the suppression of the phenotype is a loss of function mutation, and said modifier gene of SCA-1 is a suppressor gene of SCA-1.

84. The method of claim 77, wherein said modification of the phenotype associated with the ataxin-1 transgene is an enhancement of the phenotype, said mutation responsible for the enhancement of the phenotype is a gain of function mutation, and said modifier gene of SCA-1 is a suppressor gene of SCA-1.

85. A method of treating a neurodegenerative disorder, comprising:
(a) administering to a subject in need of such treatment an antagonist of a suppressor gene of SCA-1.

86. A method of treating a neurodegenerative disorder, comprising:
(a) identifying a suppressor gene of SCA-1 according to the method of claim 73 or 81; and
(b) administering to a subject in need of such treatment an antagonist of said suppressor gene of SCA-1.

87. A method of screening for a molecule having activity against a neurodegenerative disorder, comprising:
(a) screening for a molecule that antagonizes a suppressor gene of SCA-1;
wherein a molecule that antagonizes said suppressor gene of SCA-1 is molecule with activity against SCA-1.

88. A method of screening for a molecule having activity against a neurodegenerative disorder, comprising:
(a) identifying a suppressor gene of SCA-1 according to the method of claim 75 or 83; and
(b) screening for a molecule that antagonizes said suppressor gene of SCA-1;
wherein a molecule that antagonizes said suppressor gene of SCA-1 is molecule with activity against SCA-1.

89. The method of claim 85, 86, 87, or 88, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

90. The method of claim 89, wherein the neurodegenerative disorder is a polyglutamine disease.

91. The method of claim 90, wherein the polyglutamine disease is SCA-1, SCA-2, SCA-6, SCA-7, MJD, HD, SBMA, or DRPLA.

92. The method of claim 91, wherein the polyglutamine disease is SCA-1.

93. The method of claim 85 or 86, wherein the antagonist is an antisense RNA or ribozyme.

94. The method of claim 85 or 86, wherein the antagonist is an antibody, peptide, or small molecule.

95. A method of treating a neurodegenerative disorder, comprising:
(a) administering to a subject in need of such treatment an agonist of an enhancer gene of SCA-1.

96. A method of treating a neurodegenerative disorder, comprising:
(a) identifying an enhancer gene of SCA-1 according to the method of claim 75 or 83; and
(b) administering to a subject in need of such treatment an agonist of said enhancer gene of SCA-1.

97. A method of screening for a molecule with activity against a neurodegenerative disorder, comprising:
(a) screening for a molecule that agonizes an enhancer gene of SCA-1; wherein a molecule that agonizes said enhancer gene of SCA-1 is molecule with activity against SCA-1.

98. A method of screening for a molecule with activity against a neurodegenerative disorder, comprising:
(a) identifying an enhancer gene of SCA-1 according to the method of claim 73 or 81; and
(b) screening for a molecule that agonizes said enhancer gene of SCA-1; wherein a molecule that agonizes said enhancer gene of SCA-1 is molecule with activity against SCA-1.

99. The method of claim 95, 96, 97, or 98, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

100. The method of claim 99, wherein the neurodegenerative disorder is a polyglutamine disease.

101. The method of claim 100, wherein the polyglutamine disease is SCA-1, SCA-2, SCA-6, SCA-7, MJD, HD, SBMA, or DRPLA.

78. The method of claim 101, wherein the polyglutamine disease is SCA-1.

102. The method of claim 95 or 96, wherein the agonist is gene therapy vector encoding the enhancer gene of SCA-1.

103. The method of claim 102, wherein the gene therapy vector is an adenovirus, adeno-associated virus, retrovirus, or liposome.

104. A method of diagnosing a predisposition to SCA-1 in an individual, comprising:
(a) measuring the expression level of normal ataxin-1 in a sample from said individual; and
(b) determining whether said expression level is higher than normal expression of ataxin-1,
wherein a higher expression level of ataxin-1 is indicative of a predisposition to SCA-1.

105. The method of claim 104, wherein the higher level of ataxin-1 indicative of a predisposition to SCA-1 is at least 25% more than normal expression of ataxin-1.

106. The method of claim 104, wherein the higher level of ataxin-1 indicative of a predisposition to SCA-1 is at least 50% more than normal expression of ataxin-1.

107. The method of claim 104, wherein the higher level of ataxin-1 indicative of a predisposition to SCA-1 is at least 75% more than normal expression of ataxin-1.

108. The method of claim 104, wherein the higher level of ataxin-1 indicative of a predisposition to SCA-1 is at least twofold the normal expression of ataxin-1.

109. The method of claim 104, wherein ataxin-1 expression is measured by measuring ataxin-1 RNA.

110. The method of claim 104, wherein ataxin-1 expression is measured by measuring ataxin-1 protein.

111. A pharmaceutical composition for the treatment or prevention of a neurodegenerative disorder, comprising (a) a glutathione-S-transferase agonist and (b) a pharmaceutically acceptable carrier.

112. The pharmaceutical composition of claim 111, wherein the glutathione-S-transferase agonist is a nucleic acid encoding a glutathione-S-transferase protein.

113. The pharmaceutical composition of claim 112, wherein the glutathione-S-transferase protein is a theta class glutathione-S-transferase protein.

114. The pharmaceutical composition of claim 112, wherein the glutathione-S-transferase protein is a sigma class glutathione-S-transferase protein.

115. A method of treating or preventing a neurodegenerative disorder, comprising administering to an individual in the need of such treatment or prevention a glutathione-S-transferase agonist in an amount effective for the treatment or prevention of the neurodegenerative disorder.

116. The method of claim 115, wherein the glutathione-S-transferase agonist is a nucleic acid encoding a glutathione-S-transferase protein.

117. The method of claim 116, wherein the glutathione-S-transferase protein is a theta class glutathione-S-transferase protein.

118. The method of claim 116, wherein the glutathione-S-transferase protein is a sigma class glutathione-S-transferase protein.

119. A pharmaceutical composition for the treatment or prevention of a neurodegenerative disorder, comprising (a) a Sin3A agonist and (b) a pharmaceutically acceptable carrier.

120. The pharmaceutical composition of claim 119, wherein the Sin3A agonist is a nucleic acid encoding a Sin3A protein.

121. A method of treating or preventing a neurodegenerative disorder, comprising administering to an individual in the need of such treatment or prevention a Sin3A agonist in an amount effective for the treatment or prevention of the neurodegenerative disorder.

122. The method of claim 121, wherein the a Sin3A agonist is a nucleic acid encoding a Sin3A protein.

123. A pharmaceutical composition for the treatment or prevention of a neurodegenerative disorder, comprising (a) a CtBP agonist and (b) a pharmaceutically acceptable carrier.

124. The pharmaceutical composition of claim 119, wherein the CtBP agonist is a nucleic acid encoding a CtBP protein.

125. A method of treating or preventing a neurodegenerative disorder, comprising administering to an individual in the need of such treatment or prevention a CtBP agonist in an amount effective for the treatment or prevention of the neurodegenerative disorder.

126. The method of claim 125, wherein the a CtBP agonist is a nucleic acid encoding a CtBP protein.

127. A pharmaceutical composition for the treatment or prevention of a neurodegenerative disorder, comprising (a) a Trap240 agonist and (b) a pharmaceutically acceptable carrier.

128. The pharmaceutical composition of claim 127, wherein the Trap240 agonist is a nucleic acid encoding a Trap240 protein.

129. A method of treating or preventing a neurodegenerative disorder, comprising administering to an individual in the need of such treatment or prevention a Trap240 agonist in an amount effective to treat of prevent the neurodegenerative disorder.

130. The method of claim 129, wherein the Trap240 agonist is a nucleic acid encoding a Trap240 protein.

131. A pharmaceutical composition for the treatment or prevention of a neurodegenerative disorder, comprising (a) a KH-domain protein agonist and (b) a pharmaceutically acceptable carrier.

132. The pharmaceutical composition of claim 131, wherein the KH-domain protein agonist is a nucleic acid encoding a KH-domain protein.

133. A method of treating or preventing a neurodegenerative disorder, comprising administering to an individual in the need of such treatment or prevention a KH-domain protein agonist in an amount effective to treat of prevent the neurodegenerative disorder.

134. The method of claim 133, wherein the KH-domain protein agonist is a nucleic acid encoding a KH-domain protein.

135. The pharmaceutical composition of claim 111, 119, 123, 127, or 131, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

136. The pharmaceutical composition of claim 135, wherein the neurodegenerative disorder is a polyglutamine disease.

137. The pharmaceutical composition of claim 136, wherein the polyglutamine disease is SCA-1, SCA-2, SCA-6, SCA-7, MJD, HD, SBMA, or DRPLA.

138. The pharmaceutical composition of claim 137, wherein the polyglutamine disease is SCA-1.

139. The method of claim 115, 121, 125, 129, or 133, wherein the neurodegenerative disorder is a polyglutamine disease, Alzheimer's Disease, age-related loss of cognitive function, senile dementia, Parkinson's disease, amyotrophic lateral sclerosis, Wilson's Disease, cerebral palsy, progressive supranuclear palsy, Guam disease, Lewy body dementia, a prion disease, a taupathy, a spongiform encephalopathy, Creutzfeldt-Jakob disease, myotonic dystrophy, Freidrich's ataxia, ataxia, Gilles de la Tourette's syndrome, seizure disorders, epilepsy, chronic seizure disorder, stroke, brain trauma, spinal cord trauma, AIDS dementia, alcoholism, autism, retinal ischemia, glaucoma, autonomic function disorder, hypertension, neuropsychiatric disorder, schizophrenia, or schizoaffective disorder.

140. The method of claim 139, wherein the neurodegenerative disorder is a polyglutamine disease.

141. The method of claim 140, wherein the polyglutamine disease is SCA-1, SCA-2, SCA-6, SCA-7, MJD, HD, SBMA, or DRPLA.

142. The method of claim 141, wherein the polyglutamine disease is SCA-1.
